(19) United States
(12) Patent Application Publication
(10) Pub. No.: US 2022/0280598 A1
(43) Pub. Date: Sep. 8, 2022

(54) COMPLEMENT INHIBITOR DOSING REGIMENS

(71) Applicant: Apellis Pharmaceuticals, Inc., Waltham, MA (US)

(72) Inventors: Pascal Deschatelets, West Newton, MA (US); Cedric Francois, Prospect, KY (US); Federico Grossi, Needham, MA (US); Patrick Johnson, Zurich (CH); Lukas Scheibler, Telluride, CO (US)

(21) Appl. No.: 17/628,084

(22) PCT Filed: Jul. 17, 2020
(86) PCT No.: PCT/US20/42676

§ 371 Date: Jan. 18, 2022

Related U.S. Application Data
(60) Provisional application No. 62/875,932, filed on Jul. 18, 2019.

(51) Int. Cl. A61K 38/12; A61K 47/60; A61K 9/00

(52) U.S. Cl. CPC A61K 38/12; A61K 47/60; A61K 9/0019

(57) ABSTRACT

Methods and compositions for inhibiting complement are described.

### CROSS REFERENCE TO RELATED APPLICATIONS

The present application claims the benefit of U.S. Provisional Application No. 62/875,932, filed on Jul. 18, 2019, the contents of which are herein incorporated by reference in their entirety.

### BACKGROUND

Complement is an arm of the innate immune system that plays an important role in defending the body against infectious agents. The complement system comprises more than 30 serum and cellular proteins that are involved in three major pathways, known as the classical, alternative, and lectin pathways. Although compositions that inhibit complement are known, there remains a need for compositions that can acutely inhibit complement.

### SUMMARY

In one aspect, the disclosure features a method of inhibiting complement in a subject, comprising administering to a subject in need thereof about 10 mg to about 1200 mg of a PEGylated compstatin analog comprising a PEG of about 10 kD, wherein complement is inhibited or reduced (e.g., to a level that is 50%, 40%, 30%, 20%, 10%, 5%, or lower, relative to a control level) at least for about 6 hours to about 24 hours after administration, and wherein complement is not inhibited or reduced (e.g., to a level that is 50%, 40%, 30%, 20%, 10%, 5%, or lower, relative to a control level) about 12 hours to about 36 hours after administration.

In some embodiments, the method comprises administering a single dose of the PEGylated compstatin analog. In some embodiments, the single dose is a bolus. In some embodiments, the single dose is an infusion.

In some embodiments, the method comprises administering about 30 mg, about 90 mg, about 270 mg, about 540 mg, or about 600 mg of the PEGylated compstatin analog. In some embodiments, the method comprises administering the infusion at a rate of about 0.25 mg/min to about 45 mg/min. In some embodiments, the method comprises administering the infusion at a rate of about 1 mg/min, about 3 mg/min, about 9 mg/min, about 18 mg/min, or about 20 mg/min. In some embodiments, the method comprises administering the infusion over a period of about 15 minutes to about 48 hours.

In some embodiments, the method comprises administering about 30 mg of the PEGylated compstatin analog at an infusion rate of about 1 mg/min for about 30 minutes. In some embodiments, the method comprises administering about 90 mg of the PEGylated compstatin analog at an infusion rate of about 3 mg/min for about 30 minutes. In some embodiments, the method comprises administering about 270 mg of the PEGylated compstatin analog at an infusion rate of about 9 mg/min for about 30 minutes. In some embodiments, the method comprises administering about 540 mg of the PEGylated compstatin analog at an infusion rate of about 18 mg/min for about 30 minutes. In some embodiments, the method comprises administering about 540 mg of the PEGylated compstatin analog at an infusion rate of about 0.75 mg/min for about 12 hours. In some embodiments, the method comprises administering about 600 mg of the PEGylated compstatin analog at an infusion rate of about 20 mg/min for about 30 minutes.

In some embodiments, the method comprises administering two or more doses of the PEGylated compstatin analog. In some embodiments, the method comprises administering a first dose (e.g., loading dose) and a second dose (e.g., maintenance dose). In some embodiments, the first dose and the second dose comprise the same amount of the PEGylated compstatin analog. In some embodiments, the first dose and the second dose comprise different amounts of the PEGylated compstatin analog. In some embodiments, the first dose comprises about 10 mg to about 600 mg of the PEGylated compstatin analog and the second dose comprises about 10 mg to about 600 mg of the PEGylated compstatin analog. In some embodiments, the first dose comprises about 30 mg, about 90 mg, about 240 mg, about 270 mg, about 360 mg, about 540 mg, or about 600 mg of the PEGylated compstatin analog. In some embodiments, the second dose comprises about 30 mg, about 90 mg, about 240 mg, about 270 mg, about 360 mg, about 540 mg, or about 600 mg of the PEGylated compstatin analog. In some embodiments, the first dose and the second dose comprise about 30 mg, about 90 mg, about 240 mg, about 270 mg, about 360 mg, about 540 mg, or about 600 mg of the PEGylated compstatin analog.

In some embodiments, the first dose is a bolus, and the second dose is a bolus. In some embodiments, the first dose is a bolus, and the second dose is an infusion. In some embodiments, the first dose is an infusion, and the second dose is an infusion. In some embodiments, the first dose is an infusion and the second dose is a bolus.

In some embodiments, the method comprises administering the second dose at an infusion rate of about 0.25 mg/min to about 45 mg/min. In some embodiments, the method comprises administering the second dose at an infusion rate of about 0.25 mg/min, about 1 mg/min, about 3 mg/min, about 9 mg/min, about 16 mg/min, about 18 mg/min, or about 20 mg/min. In some embodiments, the method comprises administering the second dose over a period of about 15 minutes to about 48 hours. In some embodiments, the method comprises administering the second dose at about 30 mg of the PEGylated compstatin analog at an infusion rate of about 1 mg/min for about 30 minutes. In some embodiments, the method comprises administering the second dose at about 90 mg of the PEGylated compstatin analog at an infusion rate of about 3 mg/min for about 30 minutes. In some embodiments, the method comprises administering the second dose at about 240 mg of the PEGylated compstatin analog at an infusion rate of about 16 mg/min for about 15 minutes. In some embodiments, the method comprises administering the second dose at about 270 mg of the PEGylated compstatin analog at an infusion rate of about 9 mg/min for about 30 minutes. In some embodiments, the method comprises administering the second dose at about 360 mg of the PEGylated compstatin analog at an infusion rate of about 0.25 mg/min for about 24 hours. In some embodiments, the method comprises administering the second dose at about 540 mg of the PEGylated compstatin analog at an infusion rate of about 18 mg/min for about 30 minutes. In some embodiments, the method comprises administering the second dose at about 540 mg of the PEGylated compstatin analog at an infusion rate of about 0.75 mg/min for about 12 hours. In some embodiments, the method comprises administering the second dose at about 600 mg of the PEGylated compstatin analog at an infusion rate of about 20 mg/min for about 30 minutes. In some embodiments, the method comprises administering the first dose at an infusion rate of about 0.25 mg/min to about 45 mg/min. In some embodiments, the method comprises administering the first dose at an infusion rate of about 0.25 mg/min, about 1 mg/min, about 3 mg/min, about 9 mg/min, about 16 mg/min, about 18 mg/min, or about 20 mg/min. In some embodiments, the method comprises administering the first dose over a period of about 15 minutes to about 48 hours. In some embodiments, the method comprises administering the first dose at about 30 mg of the PEGylated compstatin analog at an infusion rate of about 1 mg/min for about 30 minutes. In some embodiments, the method comprises administering the first dose at about 90 mg of the PEGylated compstatin analog at an infusion rate of about 3 mg/min for about 30 minutes. In some embodiments, the method comprises comprising administering the first dose at about 240 mg of the PEGylated compstatin analog at an infusion rate of about 16 mg/min for about 15 minutes. In some embodiments, the method comprises administering the first dose at about 270 mg of the PEGylated compstatin analog at an infusion rate of about 9 mg/min for about 30 minutes. In some embodiments, the method comprises administering the first dose at about 360 mg of the PEGylated compstatin analog at an infusion rate of about 0.25 mg/min for about 24 hours. In some embodiments, the method comprises administering the first dose at about 540 mg of the PEGylated compstatin analog at an infusion rate of about 18 mg/min for about 30 minutes. In some embodiments, the method comprises administering the first dose at about 540 mg of the PEGylated compstatin analog at an infusion rate of about 0.75 mg/min for about 12 hours. In some embodiments, the method comprises administering the first dose at about 600 mg of the PEGylated compstatin analog at an infusion rate of about 20 mg/min for about 30 minutes. In some embodiments, the method comprises administering the first dose and the second dose at an infusion rate of about 0.25 mg/min to about 45 mg/min. In some embodiments, the method comprises administering the first dose and the second dose at an infusion rate of about 0.25 mg/min, about 1 mg/min, about 3 mg/min, about 9 mg/min, about 16 mg/min, about 18 mg/min, or about 20 mg/min. In some embodiments, the method comprises administering the first dose and the second dose over a period of about 15 minutes to about 48 hours. In some embodiments, the method comprises administering the first dose and the second dose at about 30 mg of the PEGylated compstatin analog at an infusion rate of about 1 mg/min for about 30 minutes. In some embodiments, the method comprises administering the first dose and the second dose at about 90 mg of the PEGylated compstatin analog at an infusion rate of about 3 mg/min for about 30 minutes. In some embodiments, the method comprises administering the first dose and the second dose at about 240 mg of the PEGylated compstatin analog at an infusion rate of about 16 mg/min for about 15 minutes. In some embodiments, the method comprises administering the first dose and the second dose at about 270 mg of the PEGylated compstatin analog at an infusion rate of about 9 mg/min for about 30 minutes. In some embodiments, the method comprises administering the first dose and the second dose at about 360 mg of the PEGylated compstatin analog at an infusion rate of about 0.25 mg/min for about 24 hours. In some embodiments, the method comprises administering the first dose and the second dose at about 540 mg of the PEGylated compstatin analog at an infusion rate of about 18 mg/min for about 30 minutes. In some embodiments, the method comprises administering the first dose and the second dose at about 540 mg of the PEGylated compstatin analog at an infusion rate of about 0.75 mg/min for about 12 hours. In some embodiments, the method comprises administering the first dose and the second dose at about 600 mg of the PEGylated compstatin analog at an infusion rate of about 20 mg/min for about 30 minutes. In some embodiments, the method comprises administering the first dose at about 240 mg of the PEGylated compstatin analog and the second dose at about 360 mg of the PEGylated compstatin analog. In some embodiments, the method comprises administering the first dose at about 240 mg of the PEGylated compstatin analog at an infusion rate of about 16 mg/min for about 15 minutes. In some embodiments, the method comprises administering the second dose at about 360 mg of the PEGylated compstatin analog at an infusion rate of about 0.25 mg/min for about 24 hours. In some embodiments, the method comprises administering the first dose at about 240 mg of the PEGylated compstatin analog at an infusion rate of about 16 mg/min for about 15 minutes and administering the second dose at about 360 mg of the PEGylated compstatin analog at an infusion rate of about 0.25 mg/min for about 24 hours.

In some embodiments, complement inhibition is assessed by measuring level of complement activity in a serum sample of the subject. In some embodiments, level of complement activity is measured using an alternative pathway assay, a classical pathway assay, or both.

In some embodiments, the PEGylated compstatin analog comprises a PEG having at least two compstatin analog moieties attached thereto. In some embodiments, the PEGylated compstatin analog comprises a linear PEG having a compstatin analog moiety attached to each end. In some embodiments, each compstatin analog moiety comprises a cyclic peptide that comprises the amino acid sequence of one of SEQ ID NOs: 3-36, 37, 69, 70, 71, and 72. In some embodiments, the PEGylated compstatin analog comprises one or more PEG moieties attached to one or more compstatin analog moieties, wherein: each compstatin analog moiety comprises a cyclic peptide having an amino acid sequence as set forth in any of SEQ ID NOs:3-36, extended by one or more terminal amino acids at the N-terminus, C-terminus, or both, wherein one or more of the amino acids has a side chain comprising a primary or secondary amine and is separated from the cyclic peptide by a rigid or flexible spacer optionally comprising an oligo(ethylene glycol) moiety; and each PEG is covalently attached via a linking moiety to one or more compstatin analog moieties, and wherein the linking moiety comprises an unsaturated alkyl moiety, a moiety comprising a nonaromatic cyclic ring system, an aromatic moiety, an ether moiety, an amide moiety, an ester moiety, a carbonyl moiety, an imine moiety, a thioether moiety, and/or an amino acid residue. In some embodiments, each compstatin analog moiety comprises a cyclic peptide extended by one or more amino acids at the N-terminus, C-terminus, or both, wherein the one or more amino acids is separated from the cyclic portion of the peptide by a rigid or flexible spacer that comprises 8-amino-3,6-dioxaoctanoic acid (AEEAc) or 11-amino-3,6,9-trioxaundecanoic acid. In some embodiments, the cyclic peptide comprises the amino acid sequence of SEQ ID NO:28, and wherein the spacer comprises AEEAc. In some embodiments, the PEGylated compstatin analog comprises the structure depicted in FIG. 1.

In some embodiments, the method comprises administering the PEGylated compstatin analog to a subject who has experienced a stroke shortly after the onset of one or more stroke symptoms, e.g., within about 5 minutes, 10 minutes, 15 minutes, 30 minutes, 1 hour, 2 hours, 3 hours, or 4 hours of onset of one or more stroke symptoms.

In some embodiments, the method comprises administering the PEGylated compstatin analog to a subject (i) prior to the start of a dialysis procedure (e.g., about 2 hours, 1 hour, 30 minutes, or 15 minutes prior to the start of dialysis) and/or (ii) during the dialysis procedure, and/or (iii) after the end of the dialysis procedure (e.g., for about 15 minutes, 30 minutes, 1 hour, or 2 hours after the end of the dialysis procedure).

In some embodiments, the method comprises administering the PEGylated compstatin analog to a subject who exhibits a sign or symptom of, or is diagnosed as having, a microangiopathy. In some embodiments, the PEGylated compstatin analog is administered for about 6 hours, 12 hours, 18 hours, 24 hours, 2 days, 3 days, or more.

In some embodiments, the method comprises administering the PEGylated compstatin analog to a subject who has or is at risk of developing autoimmune encephalitis.

In some embodiments, the method comprises administering the PEGylated compstatin analog to a subject who has received or is receiving an AAV viral vector. In some embodiments, the method comprises administering the PEGylated compstatin analog to a subject prior to receiving an AAV viral vector. In some embodiments, the method comprises administering a first dose and a second dose of the PEGylated compstatin analog, wherein the second dose is administered concurrently with an AAV viral vector.

### BRIEF DESCRIPTION OF THE DRAWING

The present teachings described herein will be more fully understood from the following description of various illustrative embodiments, when read together with the accompanying drawings. It should be understood that the drawings described below are for illustration purposes only and are not intended to limit the scope of the present teachings in any way.

FIG. 1 shows the structure of an exemplary PEGylated compstatin analog.

FIG. 2 depicts mean serum level of the PEGylated compstatin analog of FIG. 1 having a PEG of about 10 kD for the cohorts.

FIGS. 3A-3F depict C3 levels for the cohorts, where (*) indicate subjects who received placebo.

FIGS. 4A-4F depict C3a levels for the cohorts, where (*) indicate subjects who received placebo.

FIGS. 5A-5F depict CH50 levels for the cohorts, where (*) indicate subjects who received placebo.

FIGS. 6A-6F depict AH50 levels for the cohorts, where (*) indicate subjects who received placebo.

FIG. 7A depicts a representative ITC spectrogram and fit integrated data titrating 20 mM of an exemplary PEGylated compstatin analog into 4 mM C3.

FIG. 7B depicts a representative ITC spectrogram and fit integrated data titrating 20 mM of an exemplary PEGylated compstatin analog into 4 mM C3b.

FIG. 7C depicts a summary of binding of an exemplary PEGylated compstatin analog to C3 and C3b, 4 separate measurements for C3 and 3 separate measurements for C3b.

FIG. 8 depicts comparative inhibition of the alternative pathway of complement activation by an exemplary non-PEGylated compstatin analog, an exemplary compstatin analog with a PEG of about 40 kD, and a compstatin analog with a PEG of about 10 kD in human plasma. Quantification of dose-dependent inhibition of alternative pathway activation in human serum by LPS as measured by the ELISA-based Wieslab Assay; all data points in triplicate ±SE.

FIG. 9A depicts inhibition of the alternative complement activation pathway by an exemplary PEGylated compstatin analog in Cynomolgus Monkey, Human, Rabbit and Rat Serum. Quantification of concentration-dependent inhibition of alternative pathway activation by LPS in serum from cynomolgus monkey, human, rabbit or rat measured by the ELISA-based Wieslab Assay, all data points in triplicate ±SE.

FIG. 9B depicts inhibition of the classical complement activation pathway by an exemplary PEGylated compstatin analog in Cynomolgus Monkey, Human, Rabbit and Rat Serum. Quantification of concentration-dependent inhibition of classical pathway activation by human IgM in serum from cynomolgus monkey, human, rabbit or rat measured by the ELISA-based Wieslab Assay, all data points in triplicate ±SE.

### Definitions

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, a genetically-engineered animal, and/or a clone.

Antibody: As used herein, the term “antibody” refers to an immunoglobulin or a derivative thereof containing an immunoglobulin domain capable of binding to an antigen. The antibody can be of any species, e.g., human, rodent, rabbit, goat, chicken, etc. The antibody may be a member of any immunoglobulin class, including any of the human classes: IgG, IgM, IgA, IgD, and IgE, or subclasses thereof such as IgG1, IgG2, etc. In various embodiments of the invention the antibody is a fragment such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. The antibody can be monovalent, bivalent or multivalent. The antibody may be a chimeric or “humanized” antibody in which, for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16: 535-539. The antibody may be partially or completely humanized. An antibody may be polyclonal or monoclonal, though for purposes of the present invention monoclonal antibodies are generally preferred. Methods for producing antibodies that specifically bind to virtually any molecule of interest are known in the art. For example, monoclonal or polyclonal antibodies can be purified from blood or ascites fluid of an animal that produces the antibody (e.g., following natural exposure to or immunization with the molecule or an antigenic fragment thereof), can be produced using recombinant techniques in cell culture or transgenic organisms, or can be made at least in part by chemical synthesis.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Combination therapy: The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered followed by one or more additional agents. In the separate administration protocol, two or more agents may be administered a few minutes apart, or a few hours apart, a few days apart, or a few weeks apart. In some embodiments, two or more agents may be administered 1-2 weeks apart.

Complement component: As used herein, the terms “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, H, and I, and properdin, with factor H being a negative regulator of the pathway. Components of the lectin pathway include, e.g., MBL2, MASP-1, and MASP-2. Complement components also include cell-bound receptors for soluble complement components. Such receptors include, e.g., C5a receptor (C5aR), C3a receptor (C3aR), Complement Receptor 1 (CR1), Complement Receptor 2 (CR2), Complement Receptor 3 (CR3), etc. It will be appreciated that the term “complement component” is not intended to include those molecules and molecular structures that serve as “triggers” for complement activation, e.g., antigen-antibody complexes, foreign structures found on microbial or artificial surfaces, etc.

Complementary DNA: As used herein, a “complementary DNA” or “cDNA” includes recombinant polynucleotides synthesized by reverse transcription of mRNA and from which intervening sequences (introns) have been removed.

Concurrent administration: As used herein, the term “Concurrent administration” with respect to two or more agents, e.g., therapeutic agents, is administration performed using doses and time intervals such that the administered agents are present together within the body, e.g., at one or more sites of action in the body, over a time interval in non-negligible quantities. The time interval can be minutes (e.g., at least 1 minute, 1-30 minutes, 30-60 minutes), hours (e.g., at least 1 hour, 1-2 hours, 2-6 hours, 6-12 hours, 12-24 hours), days (e.g., at least 1 day, 1-2 days, 2-4 days, 4-7 days, etc.), weeks (e.g., at least 1, 2, or 3 weeks, etc.). Accordingly, the agents may, but need not be, administered together as part of a single composition. In addition, the agents may, but need not be, administered essentially simultaneously (e.g., within less than 5 minutes, or within less than 1 minute apart) or within a short time of one another (e.g., less than 1 hour, less than 30 minutes, less than 10 minutes, approximately 5 minutes apart). According to various embodiments of the disclosure, agents administered within such time intervals may be considered to be administered at substantially the same time. In certain embodiments of the disclosure, concurrently administered agents are present at effective concentrations within the body (e.g., in the blood and/or at a site of local complement activation) over the time interval. When administered concurrently, the effective concentration of each of the agents needed to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. The non-negligible concentration of an agent may be, for example, less than approximately 5% of the concentration that would be required to elicit a particular biological response, e.g., a desired biological response.

Host cell: As used herein, the term “host cell” refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, W138, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, and a cell line derived from an aforementioned cell. In some embodiments, the cell comprises one or more viral genes.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Linked: As used herein, the term “linked”, when used with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the invention the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield.

Local administration: As used herein, the term “local administration” or “local delivery”, in reference to delivery of a complement inhibitor described herein, refers to delivery that does not rely upon transport of the complement inhibitor to its intended target tissue or site via the vascular system. The complement inhibitor described herein may be delivered directly to its intended target tissue or site, or in the vicinity thereof, e.g., in close proximity to the intended target tissue or site. For example, the complement inhibitor may be delivered by injection or implantation of the composition or agent or by injection or implantation of a device containing the composition or agent. Following local administration in the vicinity of a target tissue or site, the complement inhibitor described herein, or one or more components thereof, may diffuse to the intended target tissue or site. It will be understood that once having been locally delivered a fraction of a complement inhibitor described herein (typically only a minor fraction of the administered dose) may enter the vascular system and be transported to another location, including back to its intended target tissue or site. As used herein, the term “local administration” or “local delivery”, in reference to delivery of a viral vector described herein, refers to delivery that can rely upon transport of the viral vector to its intended target tissue or site via the vascular system.

Local complement activation: As used herein, the term “local complement activation” refers to complement activation that occurs outside the vascular system.

Operably linked: As used herein, the term “operably linked” refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

Recombinant: As used herein, the term “recombinant” is intended to refer to nucleic acids or polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc) that is transgenic for or otherwise has been manipulated to express a gene or genes, or gene components that encode and/or direct expression of the polypeptide or one or more component(s), portion(s), element(s), or domain(s) thereof, and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

RNA interference: As used herein, the term “RNA interference” or “RNAi” refers generally to a process in which a double-stranded RNA molecule or a short hairpin RNA molecule reduces or inhibits expression of a nucleic acid sequence with which the double-stranded or short hairpin RNA molecule shares substantial or total homology. Without wishing to be bound by any theory, it is believed that, in nature, the RNAi pathway is initiated by a Type III endonuclease known as Dicer, which cleaves long double-stranded RNA (dsRNA) into double-stranded fragments typically of 21-23 base pairs with 2-base 3′ overhangs (although variations in length and overhangs are also contemplated), referred to as “short interfering RNAs” (“siRNAs”). Such siRNAs comprise two single-stranded RNAs (ssRNAs), with an “antisense strand” or “guide strand” that includes a region that is substantially complementary to a target sequence, and a “sense strand” or “passenger strand” that includes a region that is substantially complementary to a region of the antisense strand. Those of ordinary skill in the art will appreciate that a guide strand may be perfectly complementary to a target region of a target RNA or may have less than perfect complementarity to a target region of a target RNA.

Sequential administration: As used herein, the term “sequential administration” of two or more agents refers to administration of two or more agents to a subject such that the agents are not present together in the subject's body, or at a relevant site of activity in the body, at greater than non-negligible concentrations. Administration of the agents may, but need not, alternate. Each agent may be administered multiple times.

Subject: As used herein, the term “subject” or “test subject” refers to any organism to which a provided compound or composition is administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition has been diagnosed with and/or displays one or more symptoms of a disease, disorder, and/or condition.

Systemic: As used herein, the term “systemic” in reference to complement components, refers to complement proteins that are synthesized by liver hepatocytes and enter the bloodstream, or are synthesized by circulating macrophages or monocytes or other cells and secreted into the bloodstream.

Systemic complement activation: As used herein, the term “systemic complement activation” is complement activation that occurs in the blood, plasma, or serum and/or involves activation of systemic complement proteins at many locations throughout the body, affecting many body tissues, systems, or organs.

Systemic administration: As used herein, the term “systemic administration” and like terms are used herein consistently with their usage in the art to refer to administration of an agent such that the agent becomes widely distributed in the body in significant amounts and has a biological effect, e.g., its desired effect, in the blood and/or reaches its desired site of action via the vascular system. Typical systemic routes of administration include administration by (i) introducing the agent directly into the vascular system or (ii) subcutaneous, oral, pulmonary, or intramuscular administration wherein the agent is absorbed, enters the vascular system, and is carried to one or more desired site(s) of action via the blood.

Target gene: A “target gene”, as used herein, refers to a gene whose expression is to be modulated, e.g., inhibited. As used herein, the term “target RNA” refers to an RNA to be degraded or translationally repressed or otherwise inhibited using one or more agents, e.g., one or more miRNAs or siRNAs. A target RNA may also be referred to as a target sequence or target transcript. The RNA may be a primary RNA transcript transcribed from the target gene (e.g., a pre-mRNA) or a processed transcript, e.g., mRNA encoding a polypeptide. As used herein, the term “target portion” or “target region” refers to a contiguous portion of the nucleotide sequence of a target RNA. In some embodiments, a target portion an mRNA is at least long enough to serve as a substrate for RNA interference (RNAi)-mediated cleavage within that portion in the presence of a suitable miRNA or siRNA. A target portion may be from about 8-36 nucleotides in length, e.g., about 10-20 or about 15-30 nucleotides in length. A target portion length may have specific value or subrange within the afore-mentioned ranges. For example, in certain embodiments a target portion may be between about 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent can be an agent that, when administered to a subject, can prevent an undesired side effect, such as an immune response to a viral vector described herein. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. In some embodiments, a therapeutically effective amount of a substance is an amount that is sufficient, when administered to a subject suffering from or susceptible to a disease, disorder, and/or condition, to treat, prevent, and/or delay the onset of an undesired side effect, e.g., an immune response to a viral vector described herein. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or signs of the disease, disorder, and/or condition. In some embodiments, a therapeutically effective amount is administered in a single dose; in some embodiments, multiple unit doses are required to deliver a therapeutically effective amount.

Treating: As used herein, the term “treating” refers to providing treatment, i.e, providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a complement-mediated condition, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered to a subject who has developed a complement-mediated disorder or is at increased risk of developing such a disorder relative to a member of the general population. A composition of the disclosure can be administered prophylactically, i.e., before development of any symptom or manifestation of the condition. Typically in this case the subject will be at risk of developing the condition.

Nucleic acid: The term “nucleic acid” includes any nucleotides, analogs thereof, and polymers thereof. The term “polynucleotide” as used herein refer to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from nucleotide analogs and modified polynucleotides such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges (also referred to herein as “internucleotide linkages”). The term encompasses nucleic acids containing any combinations of nucleobases, modified nucleobases, sugars, modified sugars, phosphate bridges or modified phosphorus atom bridges. Examples include, and are not limited to, nucleic acids containing ribose moieties, the nucleic acids containing deoxyribose moieties, nucleic acids containing both ribose and deoxyribose moieties, nucleic acids containing ribose and modified ribose moieties. In some embodiments, the prefix poly- refers to a nucleic acid containing 2 to about 10,000, 2 to about 50,000, or 2 to about 100,000 nucleotide monomer units. In some embodiments, the prefix oligo- refers to a nucleic acid containing 2 to about 200 nucleotide monomer units.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” One of ordinary skill in the art understands that a “viral vector”, as described herein, includes viral components in addition to a transgene described herein, e.g., capsid proteins.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

The disclosure provides methods and compositions to inhibit complement over a defined time period, after which the level of complement inhibition is reduced relatively quickly following cessation of administration of a PEGylated compstatin analog described herein.

I. Complement System

Complement is an arm of the innate immune system that plays an important role in defending the body against infectious agents. The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylatoxins and mediate multiple reactions in the acute inflammatory response. C3a and C5a are also chemotactic factors that attract immune system cells such as neutrophils. It will be understood that the names “C2a” and “C2b” used initially were subsequently reversed in the scientific literature.

The alternative pathway is initiated by and amplified at, e.g., microbial surfaces and various complex polysaccharides. In this pathway, hydrolysis of C3 to C3 (H₂O), which occurs spontaneously at a low level, leads to binding of factor B, which is cleaved by factor D, generating a fluid phase C3 convertase that activates complement by cleaving C3 into C3a and C3b. C3b binds to targets such as cell surfaces and forms a complex with factor B, which is later cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase. C3 and C5 convertases of this pathway are regulated by cellular molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on cell surfaces prevents significant complement activation from occurring thereon.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above. Further details are found, e.g., in Kuby Immunology, 6th ed., 2006; Paul, W. E., Fundamental Immunology, Lippincott Williams & Wilkins; 6th ed., 2008; and Walport M J., Complement. First of two parts. N Engl J Med., 344(14):1058-66, 2001.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains, about 50-70 amino acids in length that contain a conserved motif including four disulfide-bonded cysteines (two disulfide bonds), proline, tryptophan, and many hydrophobic residues. The CCP family includes complement receptor type 1 (CR1; C3b:C4b receptor), complement receptor type 2 (CR2), membrane cofactor protein (MCP; CD46), decay-accelerating factor (DAF), complement factor H (fH), and C4b-binding protein (C4 bp). CD59 is a membrane-bound complement regulatory protein unrelated structurally to the CCPs. Complement regulatory proteins normally serve to limit complement activation that might otherwise occur on cells and tissues of the mammalian, e.g., human host. Thus, “self” cells are normally protected from the deleterious effects that would otherwise ensue were complement activation to proceed on these cells. Deficiencies or defects in complement regulatory protein(s) are involved in the pathogenesis of a variety of complement-mediated disorders.

II. Complement Inhibitors

(i) Compstatin Analogs

Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 1), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7(3):619-27, 1998) to refer to a peptide having the same sequence as SEQ ID NO: 2 disclosed in U.S. Pat. No. 6,319,897, but amidated at the C terminus as shown in Table 1 (SEQ ID NO: 8). The term “compstatin” is used herein consistently with such usage (i.e., to refer to SEQ ID NO: 8). Compstatin analogs that have higher complement inhibiting activity than compstatin have been developed. See, e.g., WO2004/026328 (PCT/US2003/029653), Morikis, D., et al., Biochem Soc Trans. 32 (Pt 1):28-32, 2004, Mallik, B., et al., J. Med. Chem., 274-286, 2005; Katragadda, M., et al. J. Med. Chem., 49: 4616-4622, 2006; WO2007062249 (PCT/US2006/045539); WO2007044668 (PCT/US2006/039397), WO/2009/046198 (PCT/US2008/078593); WO/2010/127336 (PCT/US2010/033345) and discussion below.

Compstatin analogs may be acetylated or amidated, e.g., at the N-terminus and/or C-terminus. For example, compstatin analogs may be acetylated at the N-terminus and amidated at the C-terminus. Consistent with usage in the art, “compstatin” as used herein, and the activities of compstatin analogs described herein relative to that of compstatin, refer to compstatin amidated at the C-terminus (Mallik, 2005, supra).

Concatamers or multimers of compstatin or a complement inhibiting analog thereof are also of use in the present invention.

As used herein, the term “compstatin analog” includes compstatin and any complement inhibiting analog thereof. The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. Certain suitable assays are described in U.S. Pat. No. 6,319,897, WO2004/026328, Morikis, supra, Mallik, supra, Katragadda 2006, supra, WO2007062249 (PCT/US2006/045539); WO2007044668 (PCT/US2006/039397), WO/2009/046198 (PCT/US2008/078593); and/or WO/2010/127336 (PCT/US2010/033345). The assay may, for example, measure alternative or classical pathway-mediated erythrocyte lysis or be an ELISA assay. In some embodiments, an assay described in WO/2010/135717 (PCT/US2010/035871) is used.

The activity of a compstatin analog may be expressed in terms of its IC₅₀ (the concentration of the compound that inhibits complement activation by 50%), with a lower IC₅₀ indicating a higher activity as recognized in the art. The activity of a preferred compstatin analog for use in the present invention is at least as great as that of compstatin. It is noted that certain modifications known to reduce or eliminate complement inhibiting activity and may be explicitly excluded from any embodiment of the invention. The IC₅₀ of compstatin has been measured as 12 μM using an alternative pathway-mediated erythrocyte lysis assay (WO2004/026328). It will be appreciated that the precise IC₅₀ value measured for a given compstatin analog will vary with experimental conditions (e.g., the serum concentration used in the assay). Comparative values, e.g., obtained from experiments in which IC₅₀ is determined for multiple different compounds under substantially identical conditions, are of use. In one embodiment, the IC₅₀ of the compstatin analog is no more than the IC₅₀ of compstatin. In certain embodiments, activity of a compstatin analog is between 2 and 99 times that of compstatin (i.e., the analog has an IC₅₀ that is less than the IC₅₀ of compstatin by a factor of between 2 and 99). For example, the activity may be between 10 and 50 times as great as that of compstatin, or between 50 and 99 times as great as that of compstatin. In certain embodiments, activity of a compstatin analog is between 99 and 264 times that of compstatin. For example, the activity may be 100, 110, 120, 130, 140, 150, 160, 170, 180, 190, 200, 210, 220, 230, 240, 250, 260, or 264 times as great as that of compstatin. In certain embodiments, the activity is between 250 and 300, 300 and 350, 350 and 400, or 400 and 500 times as great as that of compstatin. The disclosure further includes compstatin analogs having activities between 500 and 1000 times that of compstatin, or more. In certain embodiments, the IC₅₀ of the compstatin analog is between about 0.2 μM and about 0.5 μM. In certain embodiments, the IC₅₀ of the compstatin analog is between about 0.1 μM and about 0.2 μM. In certain embodiments, the IC₅₀ of the compstatin analog is between about 0.05 μM and about 0.1 μM. In certain embodiments, the IC₅₀ of the compstatin analog is between about 0.001 μM and about 0.05 μM.

The K_{d} of compstatin binding to C3 can be measured using isothermal titration calorimetry (Katragadda, et al., J. Biol. Chem., 279(53), 54987-54995, 2004). Binding affinity of a variety of compstatin analogs for C3 has been correlated with their activity, with a lower K_{d} indicating a higher binding affinity, as recognized in the art. A linear correlation between binding affinity and activity was shown for certain analogs tested (Katragadda, 2004, supra; Katragadda 2006, supra). In certain embodiments, a compstatin analog described herein binds to C3 with a K_{d} of between 0.1 μM and 1.0 μM, between 0.05 μM and 0.1 μM, between 0.025 μM and 0.05 μM, between 0.015 μM and 0.025 μM, between 0.01 μM and 0.015 μM, or between 0.001 μM and 0.01 μM.

Compounds “designed or identified based on compstatin” include, but are not limited to, compounds that comprise an amino acid chain whose sequence is obtained by (i) modifying the sequence of compstatin (e.g., replacing one or more amino acids of the sequence of compstatin with a different amino acid or amino acid analog, inserting one or more amino acids or amino acid analogs into the sequence of compstatin, or deleting one or more amino acids from the sequence of compstatin); (ii) selection from a phage display peptide library in which one or more amino acids of compstatin is randomized, and optionally further modified according to method (i); or (iii) identified by screening for compounds that compete with compstatin or any analog thereof obtained by methods (i) or (ii) for binding to C3 or a fragment thereof. Many useful compstatin analogs comprise a hydrophobic cluster, a β-turn, and a disulfide bridge.

In certain embodiments, sequence of a compstatin analog comprises or consists essentially of a sequence that is obtained by making 1, 2, 3, or 4 substitutions in the sequence of compstatin, i.e., 1, 2, 3, or 4 amino acids in the sequence of compstatin is replaced by a different standard amino acid or by a non-standard amino acid. In certain embodiments, the amino acid at position 4 is altered. In certain embodiments, the amino acid at position 9 is altered. In certain embodiments, the amino acids at positions 4 and 9 are altered. In certain embodiments, only the amino acids at positions 4 and 9 are altered. In certain embodiments, the amino acid at position 4 or 9 is altered, or in certain embodiments both amino acids 4 and 9 are altered, and in addition up to 2 amino acids located at positions selected from 1, 7, 10, 11, and 13 are altered. In certain embodiments, the amino acids at positions 4, 7, and 9 are altered. In certain embodiments, amino acids at position 2, 12, or both are altered, provided that the alteration preserves the ability of the compound to be cyclized. Such alteration(s) at positions 2 and/or 12 may be in addition to the alteration(s) at position 1, 4, 7, 9, 10, 11, and/or 13. Optionally the sequence of any of the compstatin analogs whose sequence is obtained by replacing one or more amino acids of compstatin sequence further includes up to 1, 2, or 3 additional amino acids at the C-terminus. In one embodiment, the additional amino acid is Gly. Optionally the sequence of any of the compstatin analogs whose sequence is obtained by replacing one or more amino acids of compstatin sequence further includes up to 5, or up to 10 additional amino acids at the C-terminus. It should be understood that compstatin analogs may have any one or more of the characteristics or features of the various embodiments described herein, and characteristics or features of any embodiment may additionally characterize any other embodiment described herein, unless otherwise stated or evident from the context. In certain embodiments, the sequence of a compstatin analog comprises or consists essentially of a sequence identical to that of compstatin except at positions corresponding to positions 4 and 9 in the sequence of compstatin.

Compstatin and certain compstatin analogs having somewhat greater activity than compstatin contain only standard amino acids (“standard amino acids” are glycine, leucine, isoleucine, valine, alanine, phenylalanine, tyrosine, tryptophan, aspartic acid, asparagine, glutamic acid, glutamine, cysteine, methionine, arginine, lysine, proline, serine, threonine and histidine). Certain compstatin analogs having improved activity incorporate one or more non-standard amino acids. Useful non-standard amino acids include singly and multiply halogenated (e.g., fluorinated) amino acids, D-amino acids, homo-amino acids, N-alkyl amino acids, dehydroamino acids, aromatic amino acids (other than phenylalanine, tyrosine and tryptophan), ortho-, meta- or para-aminobenzoic acid, phospho-amino acids, methoxylated amino acids, and α,α-disubstituted amino acids. In certain embodiments, a compstatin analog is designed by replacing one or more L-amino acids in a compstatin analog described elsewhere herein with the corresponding D-amino acid. Such compounds and methods of use thereof are an aspect of the disclosure. Exemplary non-standard amino acids of use include 2-naphthylalanine (2-NaI), 1-naphthylalanine (1-NaI), 2-indanylglycine carboxylic acid (2Ig1), dihydrotrpytophan (Dht), 4-benzoyl-L-phenylalanine (Bpa), 2-α-aminobutyric acid (2-Abu), 3-α-aminobutyric acid (3-Abu), 4-α-aminobutyric acid (4-Abu), cyclohexylalanine (Cha), homocyclohexylalanine (hCha), 4-fluoro-L-tryptophan (4fW), 5-fluoro-L-tryptophan (5fW), 6-fluoro-L-tryptophan (6fW), 4-hydroxy-L-tryptophan (4OH—W), 5-hydroxy-L-tryptophan (5OH—W), 6-hydroxy-L-tryptophan (6OH—W), 1-methyl-L-tryptophan (1MeW), 4-methyl-L-tryptophan (4MeW), 5-methyl-L-tryptophan (5MeW), 7-aza-L-tryptophan (7aW), α-methyl-L-tryptophan (αMeW), β-methyl-L-tryptophan (βMeW), N-methyl-L-tryptophan (NMeW), ornithine (orn), citrulline, norleucine, γ-glutamic acid, etc.

In certain embodiments, the compstatin analog comprises one or more Trp analogs (e.g., at position 4 and/or 7 relative to the sequence of compstatin). Exemplary Trp analogs are mentioned above. See also Beene, et. al. Biochemistry 41: 10262-10269, 2002 (describing, inter alia, singly- and multiply-halogenated Trp analogs); Babitzke & Yanofsky, J. Biol. Chem. 270: 12452-12456, 1995 (describing, inter alia, methylated and halogenated Trp and other Trp and indole analogs); and U.S. Pat. Nos. 6,214,790, 6,169,057, 5,776,970, 4,870,097, 4,576,750 and 4,299,838. Other Trp analogs include variants that are substituted (e.g., by a methyl group) at the α or β carbon and, optionally, also at one or more positions of the indole ring. Amino acids comprising two or more aromatic rings, including substituted, unsubstituted, or alternatively substituted variants thereof, are of interest as Trp analogs. In certain embodiments, the Trp analog, e.g., at position 4, is 5-methoxy, 5-methyl-, 1-methyl-, or 1-formyl-tryptophan. In certain embodiments, a Trp analog (e.g., at position 4) comprising a 1-alkyl substituent, e.g., a lower alkyl (e.g., C₁-C₅) substituent is used. In certain embodiments, N(α) methyl tryptophan or 5-methyltryptophan is used. In some embodiments, an analog comprising a 1-alkanoyl substituent, e.g., a lower alkanoyl (e.g., C₁-C₅) is used. Examples include 1-acetyl-L-tryptophan and L-β-tryptophan.

In certain embodiments, the Trp analog has increased hydrophobic character relative to Trp. For example, the indole ring may be substituted by one or more alkyl (e.g., methyl) groups. In certain embodiments, the Trp analog participates in a hydrophobic interaction with C3. Such a Trp analog may be located, e.g., at position 4 relative to the sequence of compstatin. In certain embodiments, the Trp analog comprises a substituted or unsubstituted bicyclic aromatic ring component or two or more substituted or unsubstituted monocyclic aromatic ring components.

In certain embodiments, the Trp analog has increased propensity to form hydrogen bonds with C3 relative to Trp but does not have increased hydrophobic character relative to Trp. The Trp analog may have increased polarity relative to Trp and/or an increased ability to participate in an electrostatic interaction with a hydrogen bond donor on C3. Certain exemplary Trp analogs with an increased hydrogen bond forming character comprise an electronegative substituent on the indole ring. Such a Trp analog may be located, e.g., at position 7 relative to the sequence of compstatin.

In certain embodiments, the compstatin analog comprises one or more Ala analogs (e.g., at position 9 relative to the sequence of compstatin), e.g., Ala analogs that are identical to Ala except that they include one or more CH₂ groups in the side chain. In certain embodiments, the Ala analog is an unbranched single methyl amino acid such as 2-Abu. In certain embodiments, the compstatin analog comprises one or more Trp analogs (e.g., at position 4 and/or 7 relative to the sequence of compstatin) and an Ala analog (e.g., at position 9 relative to the sequence of compstatin).

In certain embodiments, the compstatin analog is a compound that comprises a peptide that has a sequence of (X′aa)ₙ-Gln-Asp-Xaa-Gly-(X″aa)ₘ, (SEQ ID NO: 2) wherein each X′aa and each X″aa is an independently selected amino acid or amino acid analog, wherein Xaa is Trp or an analog of Trp, and wherein n>1 and m>1 and n+m is between 5 and 21. The peptide has a core sequence of Gln-Asp-Xaa-Gly, where Xaa is Trp or an analog of Trp, e.g., an analog of Trp having increased propensity to form hydrogen bonds with an H-bond donor relative to Trp but, in certain embodiments, not having increased hydrophobic character relative to Trp. For example, the analog may be one in which the indole ring of Trp is substituted with an electronegative moiety, e.g., a halogen such as fluorine. In one embodiment, Xaa is 5-fluorotryptophan. Absent evidence to the contrary, one of skill in the art would recognize that any non-naturally occurring peptide whose sequence comprises this core sequence and that inhibits complement activation and/or binds to C3 will have been designed based on the sequence of compstatin. In an alternative embodiment, Xaa is an amino acid or amino acid analog other than a Trp analog that allows the Gln-Asp-Xaa-Gly peptide to form a β-turn.

In certain embodiments, the peptide has a core sequence of X′aa-Gln-Asp-Xaa-Gly (SEQ ID NO: 3), where X′aa and Xaa are selected from Trp and analogs of Trp. In certain embodiments, the peptide has a core sequence of X′aa-Gln-Asp-Xaa-Gly (SEQ ID NO: 3), where X′aa and Xaa are selected from Trp, analogs of Trp, and other amino acids or amino acid analogs comprising at least one aromatic ring. In certain embodiments, the core sequence forms a β-turn in the context of the peptide. The β-turn may be flexible, allowing the peptide to assume two or more conformations as assessed for example, using nuclear magnetic resonance (NMR). In certain embodiments, X′aa is an analog of Trp that comprises a substituted or unsubstituted bicyclic aromatic ring component or two or more substituted or unsubstituted monocyclic aromatic ring components. In certain embodiments, X′aa is selected from the group consisting of 2-naphthylalanine, 1-naphthylalanine, 2-indanylglycine carboxylic acid, dihydrotryptophan, and benzoylphenylalanine. In certain embodiments, X′aa is an analog of Trp that has increased hydrophobic character relative to Trp. For example, X′aa may be 1-methyltryptophan. In certain embodiments, Xaa is an analog of Trp that has increased propensity to form hydrogen bonds relative to Trp but, in certain embodiments, not having increased hydrophobic character relative to Trp. In certain embodiments, the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan.

In certain embodiments, the peptide has a core sequence of X′aa-Gln-Asp-Xaa-Gly-X″aa (SEQ ID NO: 4), where X′aa and Xaa are each independently selected from Trp and analogs of Trp and X″aa is selected from His, Ala, analogs of Ala, Phe, and Trp. In certain embodiments, X′aa is an analog of Trp that has increased hydrophobic character relative to Trp, such as 1-methyltryptophan or another Trp analog having an alkyl substituent on the indole ring (e.g., at position 1, 4, 5, or 6). In certain embodiments, X′aa is an analog of Trp that comprises a substituted or unsubstituted bicyclic aromatic ring component or two or more substituted or unsubstituted monocyclic aromatic ring components. In certain embodiments, X′aa is selected from the group consisting of 2-naphthylalanine, 1-naphthylalanine, 2-indanylglycine carboxylic acid, dihydrotryptophan, and benzoylphenylalanine. In certain embodiments, Xaa is an analog of Trp that has increased propensity to form hydrogen bonds with C3 relative to Trp but, in certain embodiments, not having increased hydrophobic character relative to Trp. In certain embodiments, the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan. In certain embodiments, X″aa is Ala or an analog of Ala such as Abu or another unbranched single methyl amino acid. In certain embodiments, the peptide has a core sequence of X′aa-Gln-Asp-Xaa-Gly-X″aa (SEQ ID NO: 4), where X′aa and Xaa are each independently selected from Trp, analogs of Trp, and amino acids or amino acid analogs comprising at least one aromatic side chain, and X″aa is selected from His, Ala, analogs of Ala, Phe, and Trp. In certain embodiments, X″aa is selected from analogs of Trp, aromatic amino acids, and aromatic amino acid analogs.

In certain preferred embodiments, the peptide is cyclic. The peptide may be cyclized via a bond between any two amino acids, one of which is (X′aa)ₙ and the other of which is located within (X″aa)ₘ. In certain embodiments, the cyclic portion of the peptide is between 9 and 15 amino acids in length, e.g., 10-12 amino acids in length. In certain embodiments, the cyclic portion of the peptide is 11 amino acids in length, with a bond (e.g., a disulfide bond) between amino acids at positions 2 and 12. For example, the peptide may be 13 amino acids long, with a bond between amino acids at positions 2 and 12 resulting in a cyclic portion 11 amino acids in length.

In certain embodiments, the peptide comprises or consists of the sequence X′aa1-X′aa2-X′aa3-X′aa4-Gln-Asp-Xaa-Gly-X″aa1-X″aa2-X″aa3-X″aa4-X″aa5 (SEQ ID NO: 5). In certain embodiments, X′aa4 and Xaa are selected from Trp and analogs of Trp, and X′aa1, X′aa2, X′aa3, X″aa1, X″aa2, X″aa3, X″aa4, and X″aa5 are independently selected from among amino acids and amino acid analogs. In certain embodiments, X′aa4 and Xaa are selected from aromatic amino acids and aromatic amino acid analogs. Any one or more of X′aa1, X′aa2, X′aa3, X″aa1, X″aa2, X″aa3, X″aa4, and X″aa5 may be identical to the amino acid at the corresponding position in compstatin. In one embodiment, X″aa1 is Ala or a single methyl unbranched amino acid. The peptide may be cyclized via a covalent bond between (i) X′aa1, X′aa2, or X′aa3; and (ii) X″aa2, X″aa3, X″aa4 or X″aa5. In one embodiment the peptide is cyclized via a covalent bond between X′aa2 and X″aa4. In one embodiment, the covalently bound amino acid are each Cys and the covalent bond is a disulfide (S—S) bond. In other embodiments, the covalent bond is a C—C, C—O, C—S, or C—N bond. In certain embodiments, one of the covalently bound residues is an amino acid or amino acid analog having a side chain that comprises a primary or secondary amine, the other covalently bound residue is an amino acid or amino acid analog having a side chain that comprises a carboxylic acid group, and the covalent bond is an amide bond. Amino acids or amino acid analogs having a side chain that comprises a primary or secondary amine include lysine and diaminocarboxylic acids of general structure NH₂(CH₂)ₙCH(NH₂)COOH such as 2,3-diaminopropionic acid (dapa), 2,4-diaminobutyric acid (daba), and ornithine (orn), wherein n=1 (dapa), 2 (daba), and 3 (orn), respectively. Examples of amino acids having a side chain that comprises a carboxylic acid group include dicarboxylic amino acids such as glutamic acid and aspartic acid. Analogs such as beta-hydroxy-L-glutamic acid may also be used. In some embodiments, a peptide is cyclized with a thioether bond, e.g., as described in PCT/US2011/052442 (WO/2012/040259). For example, in some embodiments a disulfide bond in any of the peptides is replaced with a thioether bond. In some embodiments, a cystathionine is formed. In some embodiments, the cystathionine is a delta-cystathionine or a gamma-cystathionine. In some embodiments, a modification comprises replacement of a Cys-Cys disulfide bond between cysteines at X′aa2 and X″aa4 in SEQ ID NO: 5 (or corresponding positions in other sequences) with addition of a CH₂, to form a homocysteine at X′aa2 or X″aa4, and introduction of a thioether bond, to form a cystathionine. In one embodiment, the cystathionine is a gamma-cystathionine. In another embodiment, the cystathionine is a delta-cystathionine. Another modification in accordance with the present disclosure comprises replacement of the disulfide bond with a thioether bond without the addition of a CH₂, thereby forming a lanthionine. In some embodiments, a compstatin analog having a thioether in place of a disulfide bond has increased stability, at least under some conditions, as compared with the compstatin analog having the disulfide bond.

In certain embodiments, the compstatin analog is a compound that comprises a peptide having a sequence:

Xaa1-Cys-Val-Xaa2-Gln-Asp-Xaa2*-Gly-Xaa3-His-Arg-Cys-Xaa4 (SEQ ID NO: 6); wherein:
Xaa1 is Ile, Val, Leu, B¹-Ile, B¹-Val, B¹-Leu or a dipeptide comprising Gly-Ile or B¹-Gly-Ile, and B¹ represents a first blocking moiety;
Xaa2 and Xaa2* are independently selected from Trp and analogs of Trp;
Xaa3 is His, Ala or an analog of Ala, Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of the L-Thr, D-Thr, Ile, Val, Gly, Ala, or Asn optionally is replaced by a second blocking moiety B²; and the two Cys residues are joined by a disulfide bond. In some embodiments, Xaa4 is Leu, Nle, His, or Phe or a dipeptide selected from Xaa5-Ala and Xaa5-Asn, or a tripeptide Xaa5-Ala-Asn, wherein Xaa5 is selected from Leu, Nle, His or Phe, and wherein a carboxy terminal —OH of any of the L-Thr, D-Thr, Ile, Val, Gly, Leu, Nle, His, Phe, Ala, or Asn optionally is replaced by a second blocking moiety B²; and the two Cys residues are joined by a disulfide bond.

In other embodiments, Xaa1 is absent or is any amino acid or amino acid analog, and Xaa2, Xaa2*, Xaa3, and Xaa4 are as defined above. If Xaa1 is absent, the N-terminal Cys residue may have a blocking moiety B¹ attached thereto.

In another embodiment, Xaa4 is any amino acid or amino acid analog and Xaa1, Xaa2, Xaa2*, and Xaa3 are as defined above. In another embodiment Xaa4 is a dipeptide selected from the group consisting of: Thr-Ala and Thr-Asn, wherein the carboxy terminal —OH or the Ala or Asn is optionally replaced by a second blocking moiety B².

In any of the embodiments of the compstatin analog of SEQ ID NO: 6, Xaa2 may be Trp.

In any of the embodiments of the compstatin analog of SEQ ID NO: 6, Xaa2 may be an analog of Trp comprising a substituted or unsubstituted bicyclic aromatic ring component or two or more substituted or unsubstituted monocyclic aromatic ring components. For example, the analog of Trp may be selected from 2-naphthylalanine (2-NaI), 1-naphthylalanine (1-NaI), 2-indanylglycine carboxylic acid (Ig1), dihydrotrpytophan (Dht), and 4-benzoyl-L-phenylalanine.

In any of the embodiments of the compstatin analog of SEQ ID NO: 6, Xaa2 may be an analog of Trp having increased hydrophobic character relative to Trp. For example, the analog of Trp may be selected from 1-methyltryptophan, 4-methyltryptophan, 5-methyltryptophan, and 6-methyltryptophan. In one embodiment, the analog of Trp is 1-methyltryptophan. In one embodiment, Xaa2 is 1-methyltryptophan, Xaa2* is Trp, Xaa3 is Ala, and the other amino acids are identical to those of compstatin.

In any of the embodiments of the compstatin analog of SEQ ID NO: 6, Xaa2* may be an analog of Trp such as an analog of Trp having increased hydrogen bond forming propensity with C3 relative to Trp, which, in certain embodiments, does not have increased hydrophobic character relative to Trp. In certain embodiments the analog of Trp comprises an electronegative substituent on the indole ring. For example, the analog of Trp may be selected from 5-fluorotryptophan and 6-fluorotryptophan.

In certain embodiments of the invention Xaa2 is Trp and Xaa2* is an analog of Trp having increased hydrogen bond forming propensity with C3 relative to Trp which, in certain embodiments, does not have increased hydrophobic character relative to Trp. In certain embodiments of the compstatin analog of SEQ ID NO: 6, Xaa2 is analog of Trp having increased hydrophobic character relative to Trp such as an analog of Trp selected from 1-methyltryptophan, 4-methyltryptophan, 5-methyltryptophan, and 6-methyltryptophan, and Xaa2* is an analog of Trp having increased hydrogen bond forming propensity with C3 relative to Trp which, in certain embodiments, does not have increased hydrophobic character relative to Trp. For example, in one embodiment Xaa2 is methyltryptophan and Xaa2* is 5-fluorotryptophan.

In certain of the afore-mentioned embodiments, Xaa3 is Ala. In certain of the afore-mentioned embodiments Xaa3 is a single methyl unbranched amino acid, e.g., Abu.

The disclosure further provides compstatin analogs of SEQ ID NO: 6, as described above, wherein Xaa2 and Xaa2* are independently selected from Trp, analogs of Trp, and other amino acids or amino acid analogs that comprise at least one aromatic ring, and Xaa3 is His, Ala or an analog of Ala, Phe, Trp, an analog of Trp, or another aromatic amino acid or aromatic amino acid analog.

In certain embodiments, the blocking moiety present at the N- or C-terminus of any of the compstatin analogs described herein is any moiety that stabilizes a peptide against degradation that would otherwise occur in mammalian (e.g., human or non-human primate) blood or interstitial fluid. For example, blocking moiety B¹ could be any moiety that alters the structure of the N-terminus of a peptide so as to inhibit cleavage of a peptide bond between the N-terminal amino acid of the peptide and the adjacent amino acid. Blocking moiety B² could be any moiety that alters the structure of the C-terminus of a peptide so as to inhibit cleavage of a peptide bond between the C-terminal amino acid of the peptide and the adjacent amino acid. Any suitable blocking moieties known in the art could be used. In certain embodiments of the invention blocking moiety B¹ comprises an acyl group (i.e., the portion of a carboxylic acid that remains following removal of the —OH group). The acyl group typically comprises between 1 and 12 carbons, e.g., between 1 and 6 carbons. For example, in certain embodiments of the invention blocking moiety B¹ is selected from the group consisting of: formyl, acetyl, proprionyl, butyryl, isobutyryl, valeryl, isovaleryl, etc. In one embodiment, the blocking moiety B¹ is an acetyl group, i.e., Xaa1 is Ac-Ile, Ac-Val, Ac-Leu, or Ac-Gly-Ile.

In certain embodiments of the invention blocking moiety B² is a primary or secondary amine (—NH₂ or —NHR¹, wherein R is an organic moiety such as an alkyl group).

In certain embodiments of the invention blocking moiety B¹ is any moiety that neutralizes or reduces the positive charge that may otherwise be present at the N-terminus at physiological pH. In certain embodiments of the invention blocking moiety B² is any moiety that neutralizes or reduces the negative charge that may otherwise be present at the C-terminus at physiological pH.

In certain embodiments of the invention, the compstatin analog is acetylated or amidated at the N-terminus and/or C-terminus, respectively. A compstatin analog may be acetylated at the N-terminus, amidated at the C-terminus, and or both acetylated at the N-terminus and amidated at the C-terminus. In certain embodiments of the invention a compstatin analog comprises an alkyl or aryl group at the N-terminus rather than an acetyl group.

In certain embodiments, the compstatin analog is a compound that comprises a peptide having a sequence:

Xaa1-Cys-Val-Xaa2-Gln-Asp-Xaa2*-Gly-Xaa3-His-Arg-Cys-Xaa4 (SEQ ID NO: 7); wherein:

Xaa1 is Ile, Val, Leu, Ac-Ile, Ac-Val, Ac-Leu or a dipeptide comprising Gly-Ile or Ac-Gly-Ile;
Xaa2 and Xaa2* are independently selected from Trp and analogs of Trp;
Xaa3 is His, Ala or an analog of Ala, Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of L-Thr, D-Thr, Ile, Val, Gly, Ala, or Asn optionally is replaced by —NH₂; and the two Cys residues are joined by a disulfide bond. In some embodiments, Xaa4 is Leu, Nle, His, or Phe or a dipeptide selected from Xaa5-Ala and Xaa5-Asn, or a tripeptide Xaa5-Ala-Asn, wherein Xaa5 is selected from Leu, Nle, His or Phe, and wherein a carboxy terminal —OH of any of the L-Thr, D-Thr, Ile, Val, Gly, Leu, Nle, His, Phe, Ala, or Asn optionally is replaced by a second blocking moiety B2; and the two Cys residues are joined by a disulfide bond.

In some embodiments, Xaa1, Xaa2, Xaa2*, Xaa3, and Xaa4 are as described above for the various embodiments of SEQ ID NO: 6. For example, in certain embodiments Xaa2* is Trp. In certain embodiments, Xaa2 is an analog of Trp having increased hydrophobic character relative to Trp, e.g., 1-methyltryptophan. In certain embodiments Xaa3 is Ala. In certain embodiments Xaa3 is a single methyl unbranched amino acid.

In certain embodiments, Xaa1 is Ile and Xaa4 is L-Thr.

In certain embodiments, Xaa1 is Ile, Xaa2* is Trp, and Xaa4 is L-Thr.

The disclosure further provides compstatin analogs of SEQ ID NO: 7, as described above, wherein Xaa2 and Xaa2* are independently selected from Trp, analogs of Trp, other amino acids or aromatic amino acid analogs, and Xaa3 is His, Ala or an analog of Ala, Phe, Trp, an analog of Trp, or another aromatic amino acid or aromatic amino acid analog.

In certain embodiments of any of the compstatin analogs described herein, an analog of Phe is used rather than Phe.

Table 1 provides a non-limiting list of compstatin analogs useful in the present disclosure. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus. Unless otherwise indicated, peptides in Table 1 are amidated at the C-terminus. Bold text is used to indicate certain modifications. Activity relative to compstatin is based on published data and assays described therein (WO2004/026328, WO2007044668, Mallik, 2005; Katragadda, 2006). Where multiple publications reporting an activity were consulted, the more recently published value is used, and it will be recognized that values may be adjusted in the case of differences between assays. It will also be appreciated that in certain embodiments of the invention the peptides listed in Table 1 are cyclized via a disulfide bond between the two Cys residues when used in the therapeutic compositions and methods of the invention. Alternate means for cyclizing the peptides are also within the scope of the invention. As noted above, in various embodiments of the invention one or more amino acid(s) of a compstatin analog (e.g., any of the compstatin analogs disclosed herein) can be an N-alkyl amino acid (e.g., an N-methyl amino acid). For example, and without limitation, at least one amino acid within the cyclic portion of the peptide, at least one amino acid N-terminal to the cyclic portion, and/or at least one amino acid C-terminal to the cyclic portion may be an N-alkyl amino acid, e.g., an N-methyl amino acid. In some embodiments of the invention, for example, a compstatin analog comprises an N-methyl glycine, e.g., at the position corresponding to position 8 of compstatin and/or at the position corresponding to position 13 of compstatin. In some embodiments, one or more of the compstatin analogs in Table 1 contains at least one N-methyl glycine, e.g., at the position corresponding to position 8 of compstatin and/or at the position corresponding to position 13 of compstatin. In some embodiments, one or more of the compstatin analogs in Table 1 contains at least one N-methyl isoleucine, e.g., at the position corresponding to position 13 of compstatin. For example, a Thr at or near the C-terminal end of a peptide whose sequence is listed in Table 1 or any other compstatin analog sequence may be replaced by N-methyl Ile. As will be appreciated, in some embodiments the N-methylated amino acids comprise N-methyl Gly at position 8 and N-methyl Ile at position 13. In some embodiments the N-methylated amino acids comprise N-methyl Gly in a core sequence such as SEQ ID NO: 3 or SEQ ID NO: 4. In some embodiments the N-methylated amino acids comprise N-methyl Gly in a core sequence such as SEQ ID NO: 5, SEQ ID NO: 6, or SEQ ID NO: 7.

TABLE 1

| | | SEQ ID | Activity over |
|---|---|---|---|
| Peptide | Sequence | NO: | compstatin |
| Compstatin | H-ICVVQDWGHHRCT-CONH2 | 8 | * |
| Ac-compstatin | Ac-ICVVQDWGHHRCT-CONH2 | 9 | 3 × more |
| Ac-V4Y/H9A | Ac-ICV [symbol] QDWG [symbol] HRCT-CONH2 | 10 | 14 × more |
| Ac-V4W/H9A −OH | Ac-ICV [symbol] QDWG [symbol] HRCT-COOH | 11 | 27 × more |
| Ac-V4W/H9A | Ac-ICV [symbol] QDWG [symbol] HRCT-CONH2 | 12 | 45 × more |
| Ac-V4W/H9A/T13dT −OH | Ac-ICV [symbol] QDWG [symbol] HRC [symbol] -COOH | 13 | 55 × more |
| Ac-V4(2-Nal)/H9A | Ac-ICV( [symbol] )QDWG [symbol] HRCT-CONH2 | 14 | 99 × more |
| Ac V4(2-Nal)/H9A −OH | Ac-ICV( [symbol] )QDWG [symbol] HRCT-COOH | 15 | 38 × more |
| Ac V4(1-Nal)/H9A −OH | Ac-ICV( [symbol] )QDWG [symbol] HRCT-COH | 16 | 30 × more |
| Ac-V42Igl/H9A | Ac-ICV( [symbol] )QDWG [symbol] HRCT-CONH2 | 17 | 39 × more |
| Ac-V42Igl/H9A −OH | Ac-ICV( [symbol] )QDWG [symbol] HRCT-COOH | 18 | 37 × more |
| Ac-V4Dht/H9A −OH | Ac-ICV [symbol] QDWG [symbol] HRCT-COOH | 19 | 5 × more |
| Ac-V4(Bpa)/H9A −OH | Ac-ICV [symbol] QDWG [symbol] HRCT-COOH | 20 | 49 × more |
| Ac-V4(Bpa)/H9A | Ac-ICV [symbol] QDWG [symbol] HRCT-CONH2 | 21 | 86 × more |
| Ac-V4(Bta)/H9A −OH | Ac-ICV( [symbol] )QDWG [symbol] HRCT-COOH | 22 | 65 × more |
| Ac-V4(Bta)/H9A | Ac-ICV( [symbol] )QDWG [symbol] HRCT-CONH2 | 23 | 64 × more |
| Ac-V4W/H9(2-Abu) | Ac-ICV [symbol] QDWG(2- [symbol] )HRCT-CONH2 | 24 | 64 × more |
| +G/V4W/H9A +AN −OH | H- [symbol] CV [symbol] QDWG [symbol] HRCTA [symbol] -COOH | 25 | 38 × more |
| Ac-V4(5fW)/H9A | Ac-ICV [symbol] QDWGAHRCT-CONH₂ | 26 | 31 × more |
| Ac-V4(5-MeW)/H9A | Ac-ICV [symbol] QDWG [symbol] HRCT-CONH₂ | 27 | 67 × more |
| Ac-V4(1-MeW)/H9A | Ac-ICV [symbol] QDWG [symbol] HRCT-CONH₂ | 28 | 264 × more |
| Ac-V4W/W7(5fW)/H9A | Ac-ICV [symbol] QD [symbol] G [symbol] HRCT-CONH₂ | 29 | 121 × more |
| Ac-V4(5fW)/W7(5fW)/H9A | Ac-ICV [symbol] QD [symbol] G [symbol] HRCT-CONH₂ | 30 | NA |
| Ac-V4(5-MeW)/W7(5fW)H9A | Ac-ICV [symbol] QD [symbol] G [symbol] HRCT- | 31 | NA |
| | CONH₂ | | |
| Ac-V4(1MeW)/W7(5fW)/H9A | Ac-ICV [symbol] QD [symbol] G [symbol] HRCT- | 32 | 264 × more |
| | CONH₂ | | |
| +G/V4(6fW)/W7(6fW)H9A+N −OH | H-GICV [symbol] QD(6fW)G [symbol] HRCT [symbol] -COOH | 33 | 126 × more |
| Ac-V4(1-formyl-W)/H9A | Ac-ICV [symbol] QDWG [symbol] HRCT-CONH₂ | 34 | 264 × more |
| Ac-V4(5-methoxy-W)/H9A | Ac-ICV [symbol] QDWG [symbol] HRCT- | 35 | 76 × more |
| | CONH₂ | | |
| G/V4(5f-W)/W7(5fW)/H9A-FN −OH | H-GICV [symbol] QD [symbol] G [symbol] HRCT [symbol] -COOH | 36 | 112 × more |

NA = not available

In certain embodiments of the compositions and methods of the disclosure, the compstatin analog has a sequence selected from sequences 9-36. In certain embodiments of the compositions and methods of the disclosure, the compstatin analog has a sequence selected from SEQ ID NOs: 14, 21, 28, 29, 32, 33, 34, and 36. In certain embodiments of the compositions and/or methods of the disclosure, the compstatin analog has a sequence selected from SEQ ID NOs: 30 and 31. In one embodiment of the compositions and methods of the disclosure, the compstatin analog has a sequence of SEQ ID NO: 28. In one embodiment of the compositions and methods of the disclosure, the compstatin analog has a sequence of SEQ ID NO: 32. In one embodiment of the compositions and methods of the disclosure, the compstatin analog has a sequence of SEQ ID NO: 34. In one embodiment of the compositions and methods of the disclosure, the compstatin analog has a sequence of SEQ ID NO: 36. As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

In some embodiments, a blocking moiety B¹ comprises an amino acid, which may be represented as Xaa0. In some embodiments, blocking moiety B² comprises an amino acid, which may be represented as XaaN. In some embodiments, blocking moiety B¹ and/or B² comprises a non-standard amino acid, such as a D-amino acid, N-alkyl amino acid (e.g., N-methyl amino acid). In some embodiments, a blocking moiety B¹ and/or B² comprises a non-standard amino acid that is an analog of a standard amino acid. In some embodiments, an amino acid analog comprises a lower alkyl, lower alkoxy, or halogen substituent, as compared with a standard amino acid of which it is an analog. In some embodiments, a substituent is on a side chain. In some embodiments, a substituent is on an alpha carbon atom. In some embodiments, a blocking moiety B¹ comprising an amino acid, e.g., a non-standard amino acid, further comprises a moiety B^{1a}. For example, blocking moiety B¹ may be represented as B^{1a}-Xaa0. In some embodiments B^{1a} neutralizes or reduces a positive charge that may otherwise be present at the N-terminus at physiological pH. In some embodiments, B¹ comprises or consists of, e.g., an acyl group that, e.g., comprises between 1 and 12 carbons, e.g., between 1 and 6 carbons. In certain embodiments, blocking moiety B^{1a} is selected from the group consisting of: formyl, acetyl, proprionyl, butyryl, isobutyryl, valeryl, isovaleryl, etc. In some embodiments, a blocking moiety B² comprising an amino acid, e.g., a non-standard amino acid, may further comprise a moiety B^{2a}. For example, blocking moiety B² may be represented as XaaN-B^{2a}, where N represents the appropriate number for the amino acid (which will depend on the numbering used in the rest of the peptide). In some embodiments, B^{2a} neutralizes or reduces a negative charge that may otherwise be present at the C-terminus at physiological pH. In some embodiments, B^{2a} comprises or consists of a primary or secondary amine (e.g., NH₂). It will be understood that a blocking activity of moiety B^{1a}-Xaa0 and/or XaaN-B^{2a} may be provided by either or both components of the moiety in various embodiments. In some embodiments, a blocking moiety or portion thereof, e.g., an amino acid residue, may contribute to increasing affinity of the compound for C3 or C3b and/or improve the activity of the compound. In some embodiments, a contribution to affinity or activity of an amino acid residue may be at least as important as a contribution to blocking activity. For example, in some embodiments, Xaa0 and/or XaaN in B^{1a}-Xaa0 and/or XaaN-B^{2a} may function mainly to increase affinity or activity of the compound, while B^{1a} and/or B^{2a} may inhibit digestion of and/or neutralize a charge of the peptide. In some embodiments, a compstatin analog comprises the amino acid sequence of any of SEQ ID NOs: 5-36, wherein SEQ ID NOs: 5-36 is further extended at the N- and/or C-terminus. In some embodiments, the sequence may be represented as B^{1a}-Xaa0—SEQUENCE—XaaN-B^{2a}, where SEQUENCE represents any of SEQ ID NOs: 5-36, wherein Bia and B^{2a} may independently be present or absent. For example, in some embodiments a compstatin analog comprises B^{1a}-Xaa0-X′aa1-X′aa2-X′aa3-X′aa4-Gln-Asp-Xaa-Gly-X″aa1-X″aa2-X″aa3-X″aa4-X″aa5-XaaN-B^{2a} (SEQ ID NO: 69), where X′aa1-X′aa2-X′aa3-X′aa4, Xaa, X″aa1, X″aa2, X″aa3, X″aa4, and X″aa5 are as set forth above for SEQ ID NO: 5.

In some embodiments, a compstatin analog comprises B^{1a}-Xaa0-Xaa1-Cys-Val-Xaa2-Gln-Asp-Xaa2*-Gly-Xaa3-His-Arg-Cys-Xaa4-XaaN-B^{2a} (SEQ ID NO: 70), where Xaa1, Xaa2, Xaa2*, Xaa3, and Xaa4 are as set forth above for SEQ ID NO: 6 or wherein Xaa1, Xaa2, Xaa2*, Xaa3, and Xaa4 are as set forth for SEQ ID NO: 6 or SEQ ID NO: 7.

In some embodiments, a compstatin analog comprises B^{1a}-Xaa0-Xaa1-Xaa2-Xaa3-Xaa4-Xaa5-Xaa6-Xaa7-Xaa8-Xaa9-Xaa10-Xaa11-Xaa12-Xaa13-XaaN-B^{2a} (SEQ ID NO: 71) wherein Xaa1, Xaa2, Xaa3, Xaa4, Xaa5, Xaa6, Xaa7, Xaa8, Xaa9, Xaa10, Xaa11, Xaa12, and Xaa13 are identical to amino acids at positions 1-13 of any of SEQ ID NOs: 9-36.

In some embodiments, Xaa0 and/or XaaN in any compstatin analog sequence comprises an amino acid that comprises an aromatic ring having an alkyl substituent at one or more positions. In some embodiments, an alkyl substituent is a lower alkyl substituent. For example, in some embodiments, an alkyl substituent is a methyl or ethyl group. In some embodiments, a substituent is located at any position that does not destroy the aromatic character of the compound. In some embodiments, a substituent is located at any position that does not destroy the aromatic character of a ring to which the substituent is attached. In some embodiments, a substituent is located at position 1, 2, 3, 4, or 5. In some embodiments, Xaa0 comprises an O-methyl analog of tyrosine, 2-hydroxyphenylalanine or 3-hydroxyphenylalanine. For purposes of the present disclosure, a lower case “m” followed by a three letter amino acid abbreviation may be used to specifically indicate that the amino acid is an N-methyl amino acid. For example, where the abbreviation “mGly” appears herein, it denotes N-methyl glycine (also sometimes referred to as sarcosine or Sar). In some embodiments, Xaa0 is or comprises mGly, Tyr, Phe, Arg, Trp, Thr, Tyr(Me), Cha, mPhe, mVal, mIle, mAla, DTyr, DPhe, DArg, DTrp, DThr, DTyr(Me), mPhe, mVal, mIle, DAla, or DCha. For example, in some embodiments a compstatin analog comprises a peptide having a sequence B¹-Ile-[Cys-Val-Trp(Me)-Gln-Asp-Trp-mGly-Ala-His-Arg-Cys]-mIle-B² (SEQ ID NO: 72). The two Cys residues are joined by a disulfide bond in the active compounds. In some embodiments, the peptide is acetylated at the N-terminus and/or amidated at the C-terminus. In some embodiments, B¹ comprises B^{1a}-Xaa0 and/or B² comprises XaaN-B^{2a}, as described above. For example, in some embodiments B¹ comprises or consists of Gly, mGly, Tyr, Phe, Arg, Trp, Thr, Tyr(Me), mPhe, mVal, mIle, mAla, DTyr, DPhe, DTrp, DCha, DAla and B² comprises NH₂, e.g., a carboxy terminal —OH of mIle is replaced by NH₂. In some embodiments, B¹ comprises or consists of mGly, Tyr, DTyr, or Tyr(Me) and B² comprises NH₂, e.g., a carboxy terminal —OH of mIle is replaced by NH₂. In some embodiments, an Ile at position Xaa1 is replaced by Gly. Complement inhibition potency and/or C3b binding parameters of selected compstatin analogs are described in WO/2010/127336 (PCT/US2010/033345) and/or in Qu, et al., Immunobiology (2012), doi:10.1016/j.imbio.2012.06.003.

In some embodiments, a blocking moiety or portion thereof, e.g., an amino acid residue, may contribute to increasing affinity of the compound for C3 or C3b and/or improve the activity of the compound. In some embodiments, a contribution to affinity or activity of an amino acid or amino acid analog may be more significant than a blocking activity.

In certain embodiments of the compositions and methods of the disclosure, the compstatin analog has a sequence as set forth in Table 1, but where the Ac-group is replaced by an alternate blocking moiety B¹, as described herein. In some embodiments, the —NH₂ group is replaced by an alternate blocking moiety B², as described herein.

In one embodiment, the compstatin analog binds to substantially the same region of the β chain of human C3 as does compstatin. In one embodiment, the compstatin analog is a compound that binds to a fragment of the C-terminal portion of the β chain of human C3 having a molecular weight of about 40 kDa to which compstatin binds (Soulika, A. M., et al., Mol. Immunol., 35:160, 1998; Soulika, A. M., et al., Mol. Immunol. 43(12):2023-9, 2006). In certain embodiments, the compstatin analog is a compound that binds to the binding site of compstatin as determined in a compstatin-C3 structure, e.g., a crystal structure or NMR-derived 3D structure. In certain embodiments, the compstatin analog is a compound that could substitute for compstatin in a compstatin-C3 structure and would form substantially the same intermolecular contacts with C3 as compstatin. In certain embodiments, the compstatin analog is a compound that binds to the binding site of a peptide having a sequence set forth in Table 1, e.g., SEQ ID NO: 14, 21, 28, 29, 32, 33, 34, 36, 37, 69, 70, 71, or 72, or another compstatin analog sequence disclosed herein in a peptide-C3 structure, e.g., a crystal structure. In certain embodiments, the compstatin analog is a compound that binds to the binding site of a peptide having SEQ ID NO: 30 or 31 in a peptide-C3 structure, e.g., a crystal structure. In certain embodiments, the compstatin analog is a compound that could substitute for the peptide of SEQ ID NO: 9-36, e.g., a compound that could substitute for the peptide of SEQ ID NO: 14, 21, 28, 29, 32, 33, 34, 36, 37, 69, 70, 71, or 72, or another compstatin analog sequence disclosed herein in a peptide-C3 structure and would form substantially the same intermolecular contacts with C3 as the peptide. In certain embodiments, the compstatin analog is a compound that could substitute for the peptide of SEQ ID NO: 30 or 31 in a peptide-C3 structure and would form substantially the same intermolecular contacts with C3 as the peptide.

One of ordinary skill in the art will readily be able to determine whether a compstatin analog binds to a fragment of the C-terminal portion of the β chain of C3 using routine experimental methods. For example, one of skill in the art could synthesize a photocrosslinkable version of the compstatin analog by including a photo-crosslinking amino acid such asp-benzoyl-L-phenylalanine (Bpa) in the compound, e.g., at the C-terminus of the sequence (Soulika, A. M., et al, supra). Optionally additional amino acids, e.g., an epitope tag such as a FLAG tag or an HA tag could be included to facilitate detection of the compound, e.g., by Western blotting. The compstatin analog is incubated with the fragment and crosslinking is initiated. Colocalization of the compstatin analog and the C3 fragment indicates binding. Surface plasmon resonance may also be used to determine whether a compstatin analog binds to the compstatin binding site on C3 or a fragment thereof. One of skill in the art would be able to use molecular modeling software programs to predict whether a compound would form substantially the same intermolecular contacts with C3 as would compstatin or a peptide having the sequence of any of the peptides in Table 1, e.g., SEQ ID NO: 14, 21, 28, 29, 32, 33, 34, or 36, or in some embodiments SEQ ID NO: 30, 31, 37, 69, 70, 71, 72, or another compstatin analog sequence disclosed herein.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, WO2004026328, and/or WO2007062249. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3^{rd} ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g. to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

A compstatin analog can be modified by addition of a molecule such as polyethylene glycol (PEG) to stabilize the compound, reduce its immunogenicity, increase its lifetime in the body, increase or decrease its solubility, and/or increase its resistance to degradation. Methods for pegylation are well known in the art (Veronese, F. M. & Harris, Adv. Drug Deliv. Rev. 54, 453-456, 2002; Davis, F. F., Adv. Drug Deliv. Rev. 54, 457-458, 2002); Hinds, K. D. & Kim, S. W. Adv. Drug Deliv. Rev. 54, 505-530 (2002; Roberts, M. J., Bentley, M. D. & Harris, J. M. Adv. Drug Deliv. Rev. 54, 459-476; 2002); Wang, Y. S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, Calif., which also provides details of appropriate conjugation procedures.

In some embodiments, the compstatin analog comprises an amino acid having a side chain comprising a primary or secondary amine, e.g., a Lys residue. For example, a Lys residue, or a sequence comprising a Lys residue, is added at the N-terminus and/or C-terminus of the compstatin analog. In some embodiments, the Lys residue is separated from the cyclic portion of the compstatin analog by a rigid or flexible spacer. The spacer may, for example, comprise a substituted or unsubstituted, saturated or unsaturated alkyl chain, oligo(ethylene glycol) chain, and/or other moieties, e.g., as described herein with regard to linkers. The length of the chain may be, e.g., between 2 and 20 carbon atoms. In other embodiments the spacer is a peptide. The peptide spacer may be, e.g., between 1 and 20 amino acids in length, e.g., between 4 and 20 amino acids in length. Suitable spacers can comprise or consist of multiple Gly residues, Ser residues, or both, for example. Optionally, the amino acid having a side chain comprising a primary or secondary amine and/or at least one amino acid in a spacer is a D-amino acid. Any of a variety of polymeric backbones or scaffolds could be used. For example, the polymeric backbone or scaffold may be a polyamide, polysaccharide, polyanhydride, polyacrylamide, polymethacrylate, polypeptide, polyethylene oxide, or dendrimer. Suitable methods and polymeric backbones are described, e.g., in WO98/46270 (PCT/US98/07171) or WO98/47002 (PCT/US98/06963). In one embodiment, the polymeric backbone or scaffold comprises multiple reactive functional groups, such as carboxylic acids, anhydride, or succinimide groups. The polymeric backbone or scaffold is reacted with the compstatin analogs. In one embodiment, the compstatin analog comprises any of a number of different reactive functional groups, such as carboxylic acids, anhydride, or succinimide groups, which are reacted with appropriate groups on the polymeric backbone. Alternately, monomeric units that could be joined to one another to form a polymeric backbone or scaffold are first reacted with the compstatin analogs and the resulting monomers are polymerized. In another embodiment, short chains are prepolymerized, functionalized, and then a mixture of short chains of different composition are assembled into longer polymers.

Additional compstatin analogs are described in, e.g., WO 2012/155107 and WO 2014/078731.

(ii) PEGylated Compstatin Analogs

In some aspects of the disclosure, linker(s) are used in the production of compstatin analogs comprising a polyethylene glycol (PEG) chain that, e.g., stabilizes the compound, increases its lifetime in the body, increases its solubility, decreases its immunogenicity, and/or increases its resistance to degradation. In some embodiments, the PEG has an average molecular weight of about 10 kD In some embodiments, such a PEGylated compstatin analog has an average plasma half-life of less than 48 hours, e.g., less than 36 hours, e.g., less than 24 hours, e.g., less than 12 hours, e.g., less than 6 hours, when administered IV to humans or to non-human primates at a dose of about 10 mg to about 1200 mg. In some embodiments, average plasma half-life of a PEGylated compstatin analog following administration IV to humans or non-human primates is increased by at least a factor of 2, e.g., by a factor of 2-5, 5-10, 10-50, or 50-100-fold or 100-150-fold or 150-200 fold as compared with that of a corresponding compstatin analog having the same amino acid sequence (and, if applicable, one or more blocking moiet(ies)) but not comprising the PEG. It will be understood that in various embodiments such an increase in half-life may be observed following administration via other routes such as subcutaneous administration and/or using other doses.

In some embodiments, a compstatin analog of any of SEQ ID NOs: 3-36, 37, 69, 70, 71, or 72 is extended by one or more amino acids at the N-terminus, C-terminus, or both, wherein at least one of the amino acids has a side chain that comprises a reactive functional group such as a primary or secondary amine, a sulfhydryl group, a carboxyl group (which may be present as a carboxylate group), a guanidino group, a phenol group, an indole ring, a thioether, or an imidazole ring, which facilitate conjugation with a reactive functional group to attach a PEG to the compstatin analog. It will be understood that a corresponding compstatin analog not comprising the PEG may also lack one or more such amino acids which are present in the PEGylated compstatin analog to which it corresponds. Thus, a corresponding compstatin analog comprising any of SEQ ID NOs: 3-36, 37, 69, 70, 71, or 72 and lacking a PEG will be understood to “have the same amino acid sequence” as SEQ ID NO: 3-36, 37, 69, 70, 71, or 72, respectively. For example, a corresponding compstatin analog comprising the amino acid sequence of SEQ ID NO: 14, 21, 28, 29, 32, 33, 34, or 36 and lacking a PEG will be understood to “have the same amino acid sequence” as SEQ ID NO: 14, 21, 28, 29, 32, 33, 34, or 36, respectively.

In some embodiments, a plasma half-life is a terminal half-life after administration of a single IV dose. In some embodiments, a plasma half-life is a terminal half-life after steady state has been reached following administration of multiple IV doses. In some embodiments, a PEGylated compstatin analog achieves a Cmax in plasma at least 2-fold, 3-fold, 4-fold, or 5-fold as great as that of a corresponding compstatin analog not comprising the PEG, following administration of a single IV dose to a primate, or following administration of multiple IV doses.

In some embodiments a primate is human. In some embodiments a primate is a non-human primate, e.g., a monkey, such as a Cynomolgus monkey or Rhesus monkey.

The concentration of compstatin analog can be measured in blood and/or urine samples using, e.g., UV, HPLC, mass spectrometry (MS) or antibody to the CRM, or combinations of such methods, such as LC/MS or LC/MS/MS. Pharmacokinetic parameters such as half-life and clearance can be determined using methods known to those of ordinary skill in the art. Pharmacokinetic analysis can be performed, e.g., with WinNonlin software v 5.2 (Pharsight Corporation, St. Louis, Mo.) or other suitable programs.

In certain embodiments, a PEG is stable in physiological conditions for less than 48 hours, e.g., less than 36 hours, 24 hours, 12 hours, or less. In certain embodiments a PEG is stable in mammalian, e.g., primate, e.g., human or non-human primate (e.g., monkey) blood, plasma, or serum for less than 24 hours. In various embodiments, less than 50%, 40%, 30%, 20%, 10%, 5%, or less, of the PEG molecules remains intact upon incubation in physiological conditions for 48 hours, 36 hours, 24 hours, 12 hours, or less. In various embodiments, less than 50%, 40%, 30%, 20%, 10%, 5%, or less, of the PEG molecules remains intact upon incubation in blood, plasma, or serum at 37 degrees C. for 48 hours, 36 hours, 24 hours, 12 hours, or less. Incubation may be performed using a PEG at a concentration of between 1 microgram/ml to about 100 mg/ml in various embodiments. Samples may be analyzed at various time points. Size or intactness may be assessed using, e.g., chromatography (e.g., HPLC), mass spectrometry, Western blot, or any other suitable method. Such stability characteristics may be conferred on a moiety conjugated to the PEG. In various embodiments, a PEGylated compstatin analog may have any of the afore-mentioned stability characteristics. In some aspects intact with regard to a PEGylated compstatin analog means that the compstatin analog moiety remains conjugated to the PEG and the PEG size remains about the same as at the start of incubation or administration.

In some embodiments, a PEGylated compstatin analog has a molar activity of at least about 10%, 20%, 30%, e.g., between 30% and 40%, between 30% and 50%, between 30% and 60%, between 30% and 70%, between 30% and 80%, between 30% and 90%, or more, of the activity of a corresponding compstatin analog having the same amino acid sequence (and, if applicable, one or more blocking moiet(ies)) but not comprising a PEG. In some embodiments wherein a PEGylated compstatin analog comprises multiple compstatin analog moieties, the molar activity of the PEGylated compstatin analog is at least about 10%, 20%, or 30%, e.g., between 30% and 40%, between 30% and 50%, between 30% and 60%, between 30% and 70%, between 30% and 80%, between 30% and 90%, or more, of the sum of the activities of said compstatin analog moieties.

In some embodiments, a polyethylene glycol (PEG) comprises a (CH₂CH₂O)ₙ moiety having a molecular weight of at least 500 daltons.

In some embodiments, a linker described herein comprises an (CH₂CH₂O)ₙ moiety having an average molecular weight of about 500; 1,000; 1,500; 2,000; 5,000; 10,000; 20,000; 30,000; 40,000; 50,000; 60,000; 70,000; 80,000; 90,000; and 100,000 daltons.

In some embodiments, the average molecular weight of a PEG is about 5,000 daltons, about 10,000 daltons, about 15,000 daltons, or about 20,000 daltons. In some embodiments, the average molecular weight of the PEG is about 10,000 daltons. “Average molecular weight” refers to the number average molecular weight. In some embodiments, the polydispersity D of a (CH₂CH₂O)ₙ moiety is between 1.0005 and 1.50, e.g., between 1.005 and 1.10, 1.15, 1.20, 1.25, 1.30, 1.40, or 1.50, or any value between 1.0005 and 1.50.

In some embodiments, a (CH₂CH₂O)ₙ moiety is monodisperse and the polydispersity of a (CH₂CH₂O)ₙ moiety is 1.0. Such monodisperse (CH₂CH₂O)ₙ moieties are known in the art and are commercially available from Quanta BioDesign (Powell, Ohio), and include, by way of nonlimiting example, monodisperse moieties where n is 2, 4, 6, 8, 12, 16, 20, or 24.

In some embodiments, a compound comprises multiple (CH₂CH₂O)ₙ moieties wherein the total molecular weight of said (CH₂CH₂O)ₙ moieties is about 1,000; 5,000; 10,000; 15,000; or 20,000. In some embodiments the average total molecular weight of the compound or (CH₂CH₂O)ₙ moieties is at about 5,000 daltons, about 10,000 daltons, about 15,000 daltons, or about 20,000 daltons. In some embodiments, the compound comprises multiple (CH₂CH₂O)ₙ moieties having defined lengths, e.g., n=4, 6, 8, 10, 12, 14, 16, 18, 20, 22, 24, 26, 28, or 30 or more. In some embodiments, the compound comprises a sufficient number of (CH₂CH₂O)ₙ moieties having defined lengths to result in a total molecular weight of said (CH₂CH₂O)ₙ moieties of between about 1,000; 5,000; 10,000; 15,000; or 20,000 daltons. In some embodiments the average total molecular weight of the compound or (CH₂CH₂O)ₙ moieties is about 10,000 daltons. In some embodiments n is between about 30 and about 3000.

In some embodiments, a compstatin analog moiety is attached at each end of a linear PEG. A bifunctional PEG having a reactive functional group at each end of the chain may be used, e.g., as described herein. In some embodiments, the reactive functional groups are identical while in some embodiments different reactive functional groups are present at each end.

In some embodiments, multiple (CH₂CH₂O)ₙ moieties are provided as a branched structure. The branches may be attached to a linear polymer backbone (e.g., as a comb-shaped structure) or may emanate from one or more central core groups, e.g., as a star structure. In some embodiments, a branched molecule has 3 to 10 (CH₂CH₂O)ₙ chains. In some embodiments, a branched molecule has 4 to 8 (CH₂CH₂O)ₙ chains. In some embodiments, a branched molecule has 10, 9, 8, 7, 6, 5, 4, or 3 (CH₂CH₂O)ₙ chains. In some embodiments, a star-shaped molecule has 10-100, 10-50, 10-30, or 10-20 (CH₂CH₂O)ₙ chains emanating from a central core group. In some embodiments a PEGylated compstatin analog thus may comprise, e.g., 3-10 compstatin analog moieties, e.g., 4-8 compstatin analog moieties, each attached to a (CH₂CH₂O)ₙ chain via a functional group at the end of the chain. In some embodiments a PEGylated compstatin analog may comprise, e.g., 10-100 compstatin analog moieties, each attached to a (CH₂CH₂O)ₙ chain via a functional group at the end of the chain. In some embodiments, branches (sometimes referred to as “arms”) of a branched or star-shaped PEG contain about the same number of (CH₂CH₂O) moieties. In some embodiments, at least some of the branch lengths may differ. It will be understood that in some embodiments one or more (CH₂CH₂O)ₙ chains does not have a compstatin analog moiety attached thereto. In some embodiments at least about 30%, 40%, 50%, 60%, 70%, 80%, 90%, 95%, or 100% of the chains has a compstatin analog moiety attached thereto.

In general and compounds depicted herein, a polyethylene glycol moiety is drawn with the oxygen atom on the right side of the repeating unit or the left side of the repeating unit. In cases where only one orientation is drawn, the present invention encompasses both orientations (i.e., (CH₂CH₂O)ₙ and (OCH₂CH₂)ₙ) of polyethylene glycol moieties for a given compound or genus, or in cases where a compound or genus contains multiple polyethylene glycol moieties, all combinations of orientations are encompasses by the present disclosure.

Formulas of some exemplary monofunctional PEGs comprising a reactive functional group are illustrated below. For illustrative purposes, formulas in which the reactive functional group(s) comprise an NHS ester are depicted, but other reactive functional groups could be used, e.g., as described above. In some embodiments, the (CH₂CH₂O)ₙ are depicted as terminating at the left end with a methoxy group (OCH₃) but it will be understood that the chains depicted below and elsewhere herein may terminate with a different OR moiety (e.g., an aliphatic group, an alkyl group, a lower alkyl group, or any other suitable PEG end group) or an OH group. It will also be appreciated that moieties other than those depicted may connect the (CH₂CH₂O)ₙ moieties with the NHS group in various embodiments.

In some embodiments, a monofunctional PEG is of formula A:

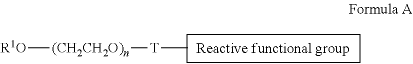

wherein “Reactive functional group” and n are as defined above and described in classes and subclasses herein;
R¹ is hydrogen, aliphatic, or any suitable end group; and
T is a covalent bond or a C1-12 straight or branched, hydrocarbon chain wherein one or more carbon units of T are optionally and independently replaced by —O—, —S—, —N(R^{x})—, —C(O)—, —C(O)O—, —OC(O)—, —N(R^{x})C(O)—, —C(O)N(R^{x})—, —S(O)—, —S(O)₂—, —N(R^{x})SO₂—, or —SO₂N(R^{x})—; and
each R^{x} is independently hydrogen or C₁₋₆ aliphatic.

Exemplary monofunctional PEGs of formula A include:

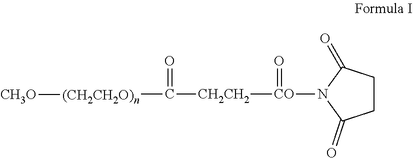

In Formula I, the moiety comprising the reactive functional group has the general structure —CO—(CH₂)ₘ—COO—NHS, where m=2. In some embodiments, a monofunctional PEGs has the structure of Formula I, where m is between 1 and 10, e.g., between 1 and 5. For example, in some embodiments m is 3, as shown below:

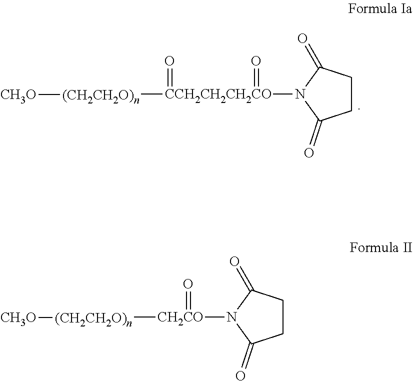

In Formula II, the moiety comprising the reactive functional group has the general structure —(CH₂)ₘ—COO—NHS, where m=1. In some embodiments a monofunctional PEG has the structure of Formula II, where m is between 1 and 10 (e.g., wherein m is 5 as shown in Formula III below), or wherein m is 0 (as shown below in Formula IIIa).

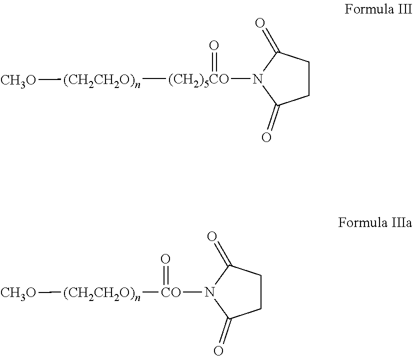

In some embodiments a bifunctional linear PEG comprises a moiety comprising a reactive functional group at each of its ends. The reactive functional groups may be the same (homobifunctional) or different (heterobifunctional). In some embodiments the structure of a bifunctional PEG may be symmetric, wherein the same moiety is used to connect the reactive functional group to oxygen atoms at each end of the —(CH₂CH₂O)ₙ chain. In some embodiments different moieties are used to connect the two reactive functional groups to the PEG portion of the molecule. The structures of exemplary bifunctional PEGs are depicted below. For illustrative purposes, formulas in which the reactive functional group(s) comprise an NHS ester are depicted, but other reactive functional groups could be used.

In some embodiments, a bifunctional linear PEG is of formula B:

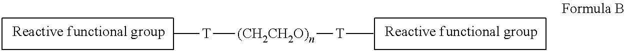

wherein each T and “Reactive functional group” is independently as defined above and described in classes and subclasses herein, and n is as defined above and described in classes and subclasses herein.

Exemplary Bifunctional PEGs of Formula B Include:

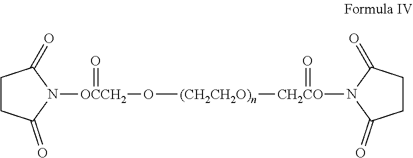

In Formula IV, the moiety comprising the reactive functional group has the general structure —(CH₂)ₘ—COO—NHS, where m=1. In some embodiments, a bifunctional PEG has the structure of Formula IV, where m is between 1 and 10, e.g., between 1 and 5. In certain embodiments m is 0, e.g., embodiments the moiety comprising the reactive functional group has the general structure —COO—NHS. For example, in some embodiments a bifunctional PEG has the structure of Formula IVa, as shown below:

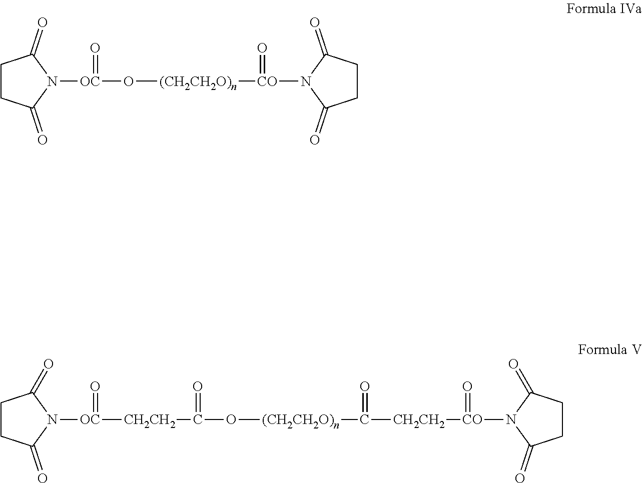

In Formula V, the moiety comprising the reactive functional group has the general structure —CO—(CH₂)ₘ—COO—NHS, where m=2. In some embodiments, a bifunctional PEGs has the structure of Formula V, where m is between 1 and 10, e.g., between 1 and 5. In certain embodiments, for example, m is 2, as shown below:

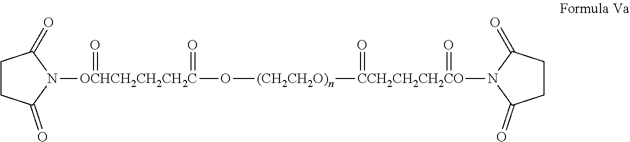

In some embodiments, a functional group (for example, an amine, hydroxyl, or thiol group) on a compstatin analog is reacted with a PEG-containing compound having a “reactive functional group” as described herein, to generate such conjugates. By way of example, Formulae III and IV, respectively, can form compstatin analog conjugates having the structure:

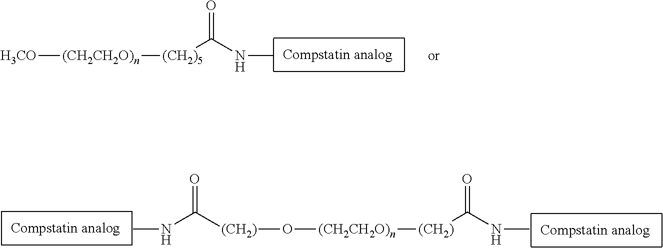

wherein

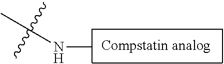 [SMILES: *NC]

represents the attachment point of an amine group on a compstatin analog. In certain embodiments, an amine group is a lysine side chain group.

It will be appreciated that corresponding conjugates can be formed with any of the PEG-containing compounds and genera depicted herein, depending on the choice of reactive functional group and/or compstatin functional group. For example, Formulae IVa and Va, respectively, can form compstatin analog conjugates having the following structures

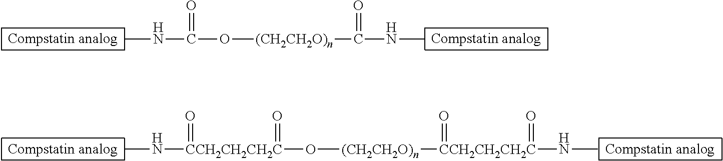

In certain embodiments, the PEG component of such conjugates has an average molecular weight of about 5 kD, about 10 kD, about 15 kD, or about 20 kD. In certain embodiments, the PEG component of such conjugates has an average molecular weight of about 10 kD.

The term “bifunctional” or “bifunctionalized” is sometimes used herein to refer to a compound comprising two compstatin analog moieties linked to a PEG. Such compounds may be designated with the letter “BF”. In some embodiments a bifunctionalized compound is symmetrical. In some embodiments the linkages between the PEG and each of the compstatin analog moieties of a bifunctionalized compound are the same. In some embodiments, each linkage between a PEG and a compstatin analog of a bifunctionalized compound comprises a carbamate. In some embodiments, each linkage between a PEG and a compstatin analog of a bifunctionalized compound comprises a carbamate and does not comprise an ester. In some embodiments, each compstatin analog of a bifunctionalized compound is directly linked to a PEG via a carbamate. In some embodiments, each compstatin analog of a bifunctionalized compound is directly linked to a PEG via a carbamate, and the bifunctionalized compound has the structure:

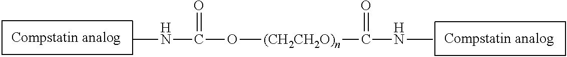

In some embodiments of formulae and embodiments described herein,

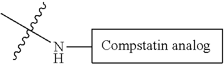 [SMILES: *NC]

represents point of attachment of a lysine side chain group in a compstatin analog having the structure:

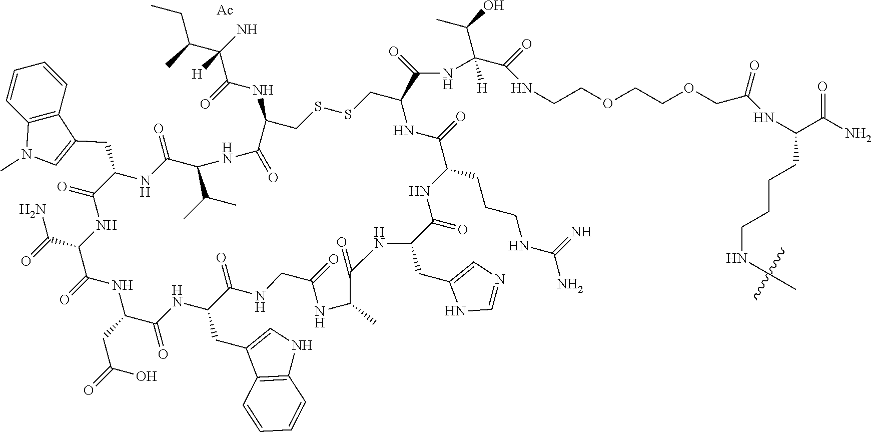 [SMILES: *NCCCC[C@H](NC(=O)COCCOCCNC(=O)[C@@H](NC(=O)[C@@H]1CSSC[C@H](NC(=O)[C@@H](N)[C@@H](C)CC)C(=O)N[C@@H](C(C)C)C(=O)N[C@@H](Cc2cn(C)c3ccccc23)C(=O)N[C@@H](C(N)=O)C(=O)N[C@@H](CC(=O)O)C(=O)N[C@@H](Cc2c[nH]c3ccccc23)C(=O)NCC(=O)N[C@@H](C)C(=O)N[C@@H](Cc2cnc[nH]2)C(=O)N[C@@H](CCCNC(=N)N)C(=O)N1)[C@@H](C)O)C(N)=O ; "Ac"]

wherein the symbol “ [symbol] ” denotes the point of attachment of a chemical moiety to the remainder of a molecule or chemical formula.

In some embodiments, a branched, comb, or star-shaped PEG comprises a moiety comprising a reactive functional group at the end of each of multiple —(CH₂CH₂O)ₙ chains. The reactive functional groups may be the same or there may be at least two different groups. In some embodiments, a branched, comb, or star-shaped PEG is of the following formulae:

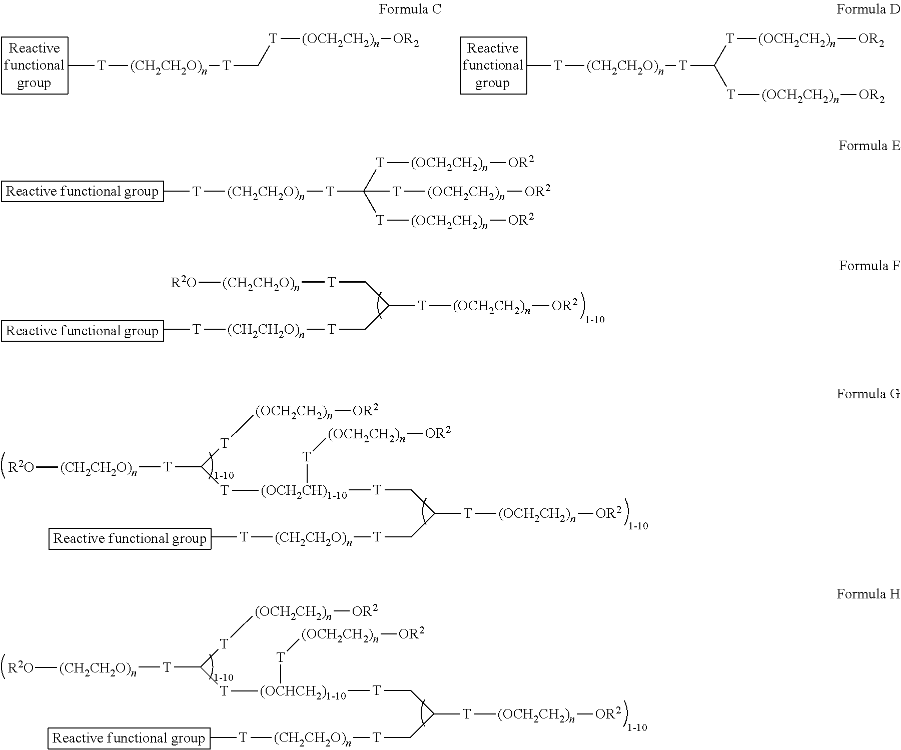

wherein each R² is independently a “Reactive functional group” or R¹, and each T, n, and “Reactive functional group” is independently as defined above and described in classes and subclasses herein. The structure of exemplary branched PEGs (having 8 arms, or branches) comprising NHS moieties as reactive functional groups is depicted below:

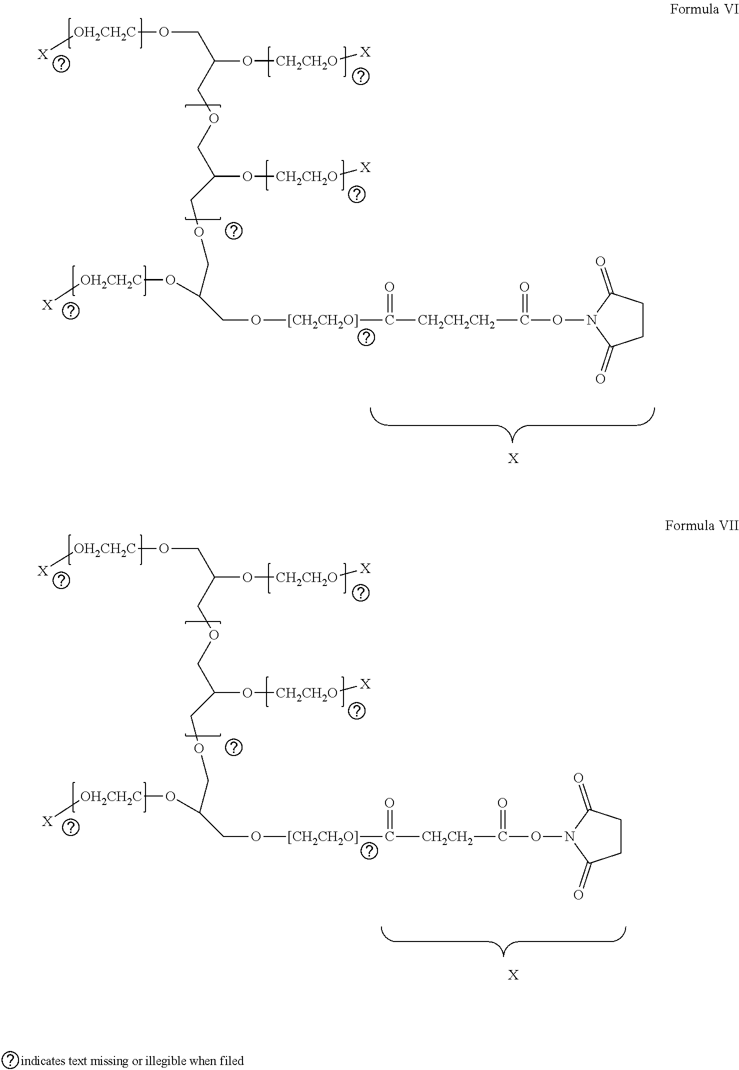

The structure of exemplary branched PEGs (having 4 arms, or branches) comprising NHS moieties as reactive functional groups is depicted below:

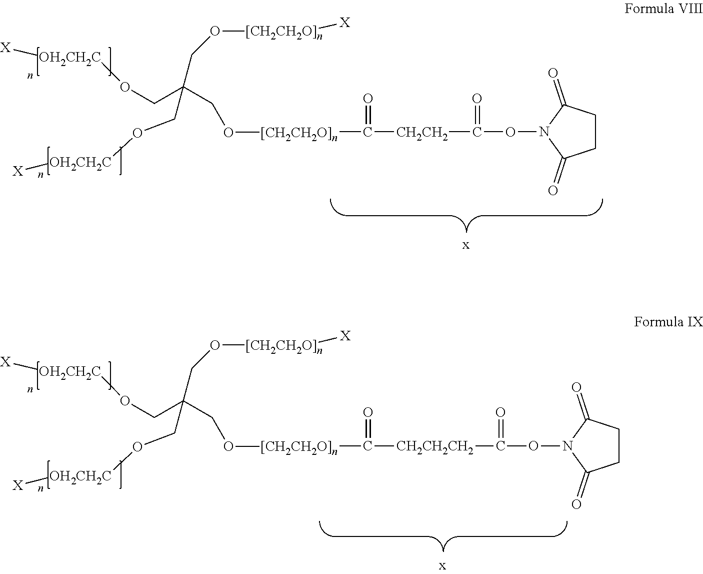

The number of branches emanating from the backbone may be varied. For example, the number 4 in the above formulae VI and VII may be changed to any other integer between 0 and 10 in various embodiments. In certain embodiments, one or more branches does not contain a reactive function group and the branch terminates with a —CH₂CH₂OH or —CH₂CH₂OR group, as described above.

In some embodiments a branched PEG has the structure of Formula VII, VIII, or IX (or variants thereof having different numbers of branches) with the proviso that x is

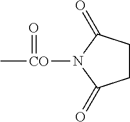

In some embodiments a branched PEG has the structure of Formula VII, VIII, or IX (or variants thereof having different numbers of branches) with the proviso that x is

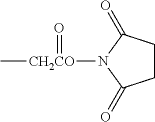

Of course the methylene (CH₂) group in the above x moiety may instead comprise a longer alkyl chain (CH₂)ₘ, where m is up to 2, 3, 4, 5, 6, 8, 10, 20, or 30, or may comprise one or more other moieties described herein.

In some embodiments, exemplary branched PEGs having NHS or maleimide reactive groups are depicted below:

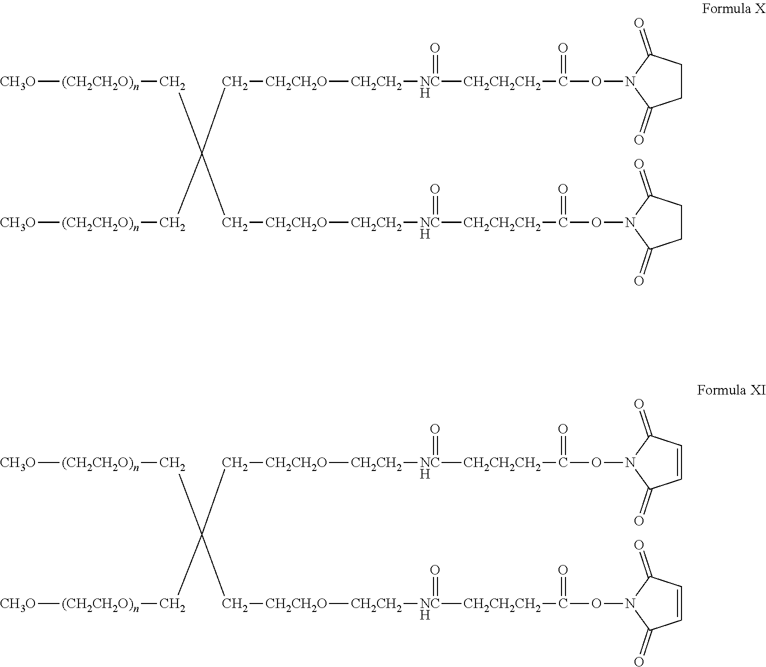

In some embodiments, a variant of Formula X or XI are used, wherein 3 or each of the 4 branches comprise a reactive functional group.

Still other examples of PEGs may be represented as follows:

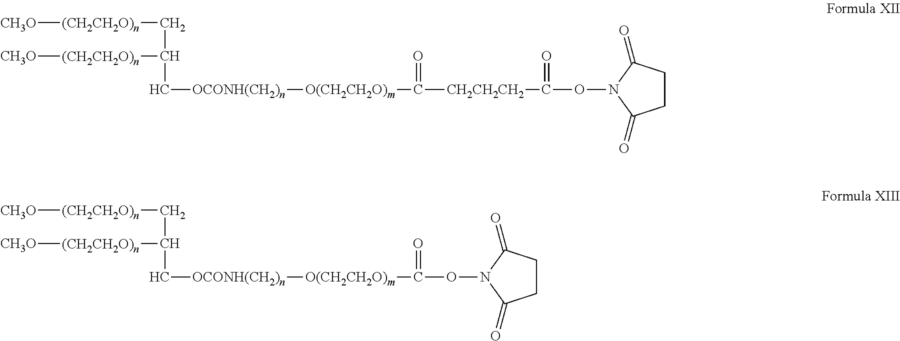

As noted above, it will be appreciated that, as described herein, in various embodiments any of a variety of moieties may be incorporated between the peptide component and (CH₂CH₂O)ₙ—R moiety of a PEGylated compstatin analog, such as an linear alkyl, ester, amide, aromatic ring (e.g., a substituted or unsubstituted phenyl), a substituted or unsubstituted cycloalkyl structure, or combinations thereof. In some embodiments such moiet(ies) may render the compound more susceptible to hydrolysis, which may release the peptide portion of the compound from the PEG. In some embodiments, such release may enhance the in vivo tissue penetration and/or activity of the compound. In some embodiments hydrolysis is general (e.g., acid-base) hydrolysis. In some embodiments hydrolysis is enzyme-catalyzed, e.g., esterase-catalyzed. Of course both types of hydrolysis may occur. Examples of PEGs comprising one or more such moieties and an NHS ester as a reactive functional group are as follows:

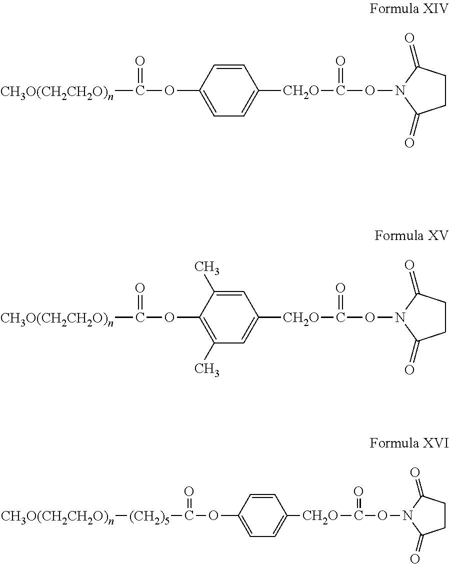

In some embodiments a branched (multi-arm) PEG or star-shaped PEG comprises a pentaerythritol core, hexaglycerin core, or tripentaerythritol core. It will be understood that the branches may not all emanate from a single point in certain embodiments.

Monofunctional, bifunctional, branched, and other PEGs comprising one or more reactive functional groups may, in some embodiments, be obtained from, e.g., NOF America Corp. White Plains, N.Y. or BOC Sciences 45-16 Ramsey Road Shirley, N.Y. 11967, USA, among others, or may be prepared using methods known in the art.

In some embodiments, a linkage between a PEG and a compstatin analog comprises a carbamate. In some embodiments, a compstatin analog is directly linked to a PEG via a carbamate. In some embodiments, a linkage between a PEG and a compstatin analog does not comprise an ester. In some embodiments, a linkage between a PEG and a compstatin analog comprises a carbamate and does not comprise an ester. In some embodiments, a linkage between a PEG and a compstatin analog comprises a carbamate and does not comprise a bond that is more susceptible to hydrolysis in aqueous medium than a carbamate.

In some embodiments, a linkage between a PEG and a compstatin analog comprises an amide. In some embodiments, a compstatin analog is directly linked to a PEG via an amide. In some embodiments, a linkage between a PEG and a compstatin analog comprises an amide and does not comprise an ester. In some embodiments, a linkage between a PEG and a compstatin analog comprises an amide and does not comprise a bond that is more susceptible to hydrolysis in aqueous medium than an amide.

In some embodiments, one or more compstatin analogs of a multifunctionalized compound (e.g., a bifunctionalized, trifunctionalized, or more extensively functionalized compound) is linked to a PEG by a linkage comprising a carbamate. In some embodiments, one or more compstatin analogs of a multifunctionalized compound (e.g., a bifunctionalized, trifunctionalized, or more extensively functionalized compound) is linked to a PEG by a linkage that does not comprise an ester. In some embodiments, one or more compstatin analogs of a multifunctionalized compound (e.g., a bifunctionalized, trifunctionalized, or more extensively functionalized compound) is linked to a PEG by a linkage that comprises a carbamate and does not comprise an ester. In some embodiments, one or more compstatin analogs of a multifunctionalized compound (e.g., a bifunctionalized, trifunctionalized, or more extensively functionalized compound) is linked to a PEG by a linkage that comprises a carbamate and does not comprise a bond that is more susceptible to hydrolysis in aqueous medium than a carbamate. In some embodiments, each compstatin analog of a multifunctionalized compound (e.g., a bifunctionalized, trifunctionalized, or more extensively functionalized compound) is directly linked to a PEG via a carbamate.

In some embodiments, one or more compstatin analogs of a multifunctionalized compound (e.g., a bifunctionalized, trifunctionalized, or more extensively functionalized compound) is linked to a PEG by a linkage comprising an amide. In some embodiments, one or more compstatin analogs of a multifunctionalized compound (e.g., a bifunctionalized, trifunctionalized, or more extensively functionalized compound) is linked to a PEG by a linkage that comprises an amide and does not comprise an ester. In some embodiments, one or more compstatin analogs of a multifunctionalized compound (e.g., a bifunctionalized, trifunctionalized, or more extensively functionalized compound) is linked to a PEG by a linkage that comprises an amide and does not comprise a bond that is more susceptible to hydrolysis in aqueous medium than an amide. In some embodiments, each compstatin analog of a multifunctionalized compound (e.g., a bifunctionalized, trifunctionalized, or more extensively functionalized compound) is directly linked to a PEG via an amide.

In some embodiments, the present invention provides compstatin analog conjugates of PEG-containing compounds and genera depicted herein, wherein the compstatin analog is connected to the PEG-containing moieties via one or more linkers. Mono- and poly-functional PEGs that comprise one or more reactive functional groups for conjugation are defined above and described in classes and subclasses herein, including but not limited to those of formula A, I, Ia, II, III, IIIa, B, IV, IVa, V, Va, C, D, E, F, G, H, VI, VII, VIII, IX, X, XI, XII, XIII, XIV, XV, or XVI.

Suitable linkers for connecting a compstatin analog and a PEG are extensively described above and in classes and subclasses herein. In some embodiments, a linker has multiple functional groups, wherein one functional group is connected to a compstatin analog and another is connected to a PEG moiety. In some embodiments, a linker is a bifunctional compound. In some embodiments, a linker has the structure of NH₂(CH₂CH₂O)nCH₂C(═O)OH, wherein n is 1 to 1000. In some embodiments, a linker is 8-amino-3,6-dioxaoctanoic acid (AEEAc). In some embodiments, a linker is activated for conjugation with a polymer moiety or a functional group of a compstatin analog. For example, in some embodiments, the carboxyl group of AEEAc is activated before conjugation with the amine group of the side chain of a lysine group.

In some embodiments, a suitable functional group (for example, an amine, hydroxyl, thiol, or carboxylic acid group) on a compstatin analog is used for conjugation with a PEG moiety, either directly or via a linker. In some embodiments, a compstatin analog is conjugated through an amine group to a PEG moiety via a linker. In some embodiments, an amine group is the α-amino group of an amino acid residue. In some embodiments, an amine group is the amine group of the lysine side chain. In some embodiments, a compstatin analog is conjugated to a PEG moiety through the amino group of a lysine side chain (s-amino group) via a linker having the structure of NH₂(CH₂CH₂O)nCH₂C(═O)OH, wherein n is 1 to 1000. In some embodiments, a compstatin analog is conjugated to the PEG moiety through the amino group of a lysine side chain via an AEEAc linker. In some embodiments, the NH₂(CH₂CH₂O)nCH₂C(═O)OH linker introduces a —NH(CH₂CH₂O)nCH₂C(═O)— moiety on a compstatin lysine side chain after conjugation. In some embodiments, the AEEAc linker introduces a —NH(CH₂CH₂O)₂CH₂C(═O)— moiety on a compstatin lysine side chain after conjugation.

In some embodiments, a compstatin analog is conjugated to a PEG moiety via a linker, wherein the linker comprises an AEEAc moiety and an amino acid residue. In some embodiments, a compstatin analog is conjugated to a PEG moiety via a linker, wherein the linker comprises an AEEAc moiety and a lysine residue. In some embodiments, the C-terminus of a compstatin analog is connected to the amino group of AEEAc, and the C-terminus of AEEAc is connected to a lysine residue. In some embodiments, the C-terminus of a compstatin analog is connected to the amino group of AEEAc, and the C-terminus of AEEAc is connected to the α-amino group of a lysine residue. In some embodiments, the C-terminus of a compstatin analog is connected to the amino group of AEEAc, the C-terminus of AEEAc is connected to the α-amino group of the lysine residue, and a PEG moiety is conjugated through the F-amino group of said lysine residue. In some embodiments, the C-terminus of the lysine residue is modified. In some embodiments, the C-terminus of the lysine residue is modified by amidation. In some embodiments, the N-terminus of a compstatin analog is modified. In some embodiments, the N-terminus of a compstatin analog is acetylated.

Exemplary conjugates comprising an AEEAc linker and a PEG are depicted below, wherein

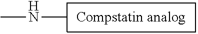

represents the attachment point of an amine group on a compstatin analog,

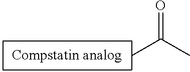

represents a compstatin analog attaching through its C-terminus, and wherein each of the other variables is independently as defined above and described in classes and subclasses herewith. In some embodiments, an amine group is the amino group of a lysine side chain.

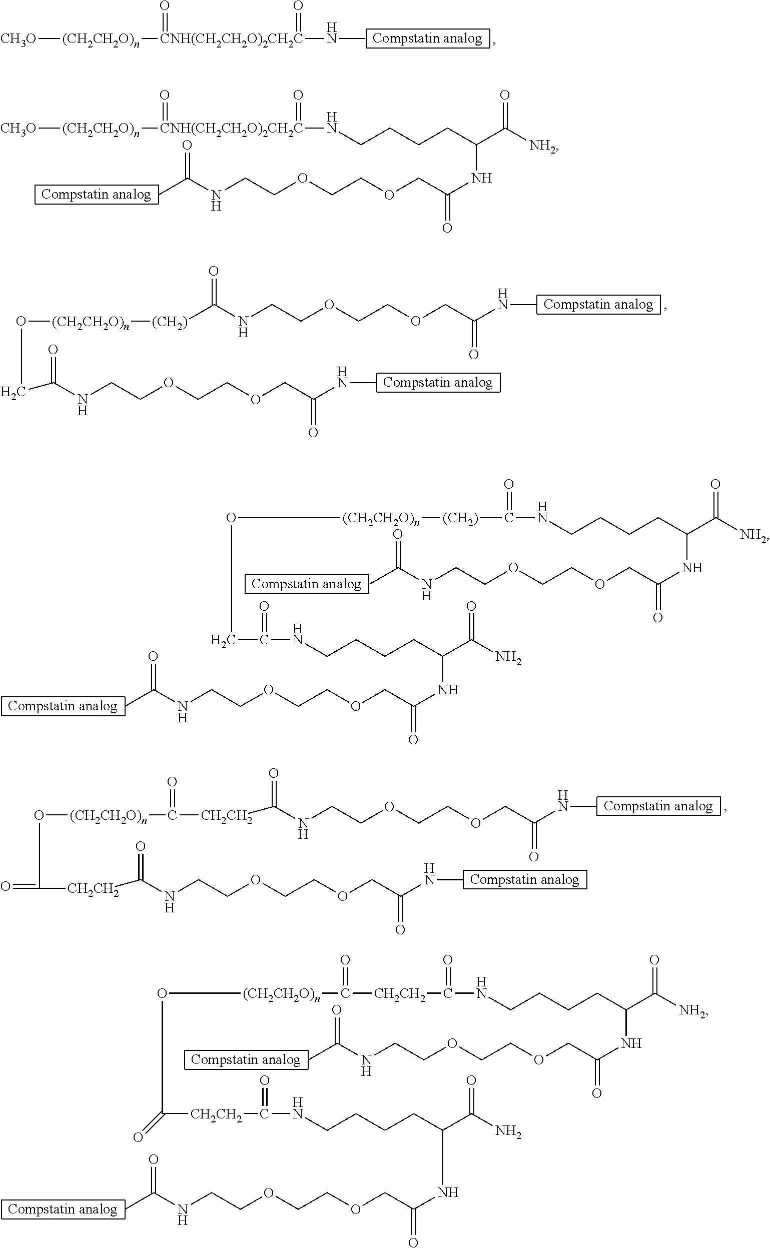

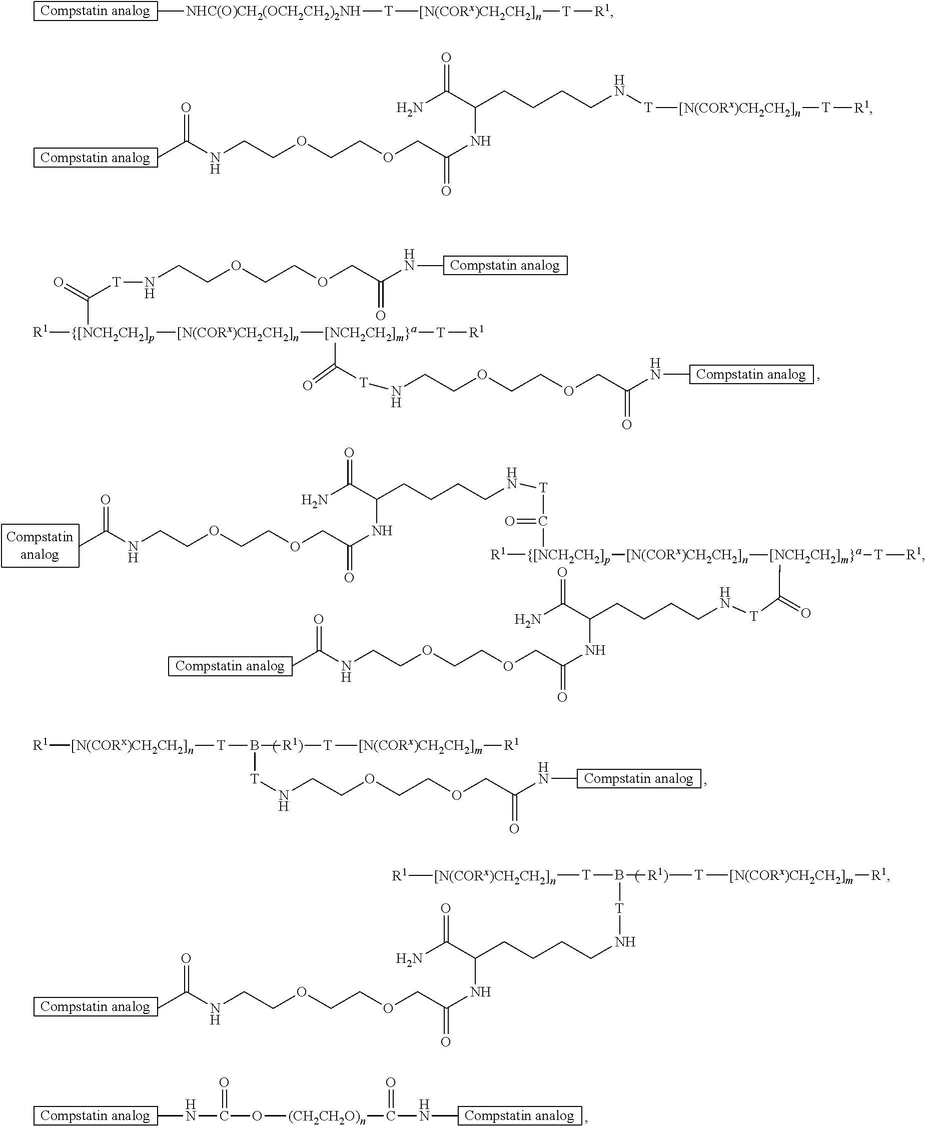

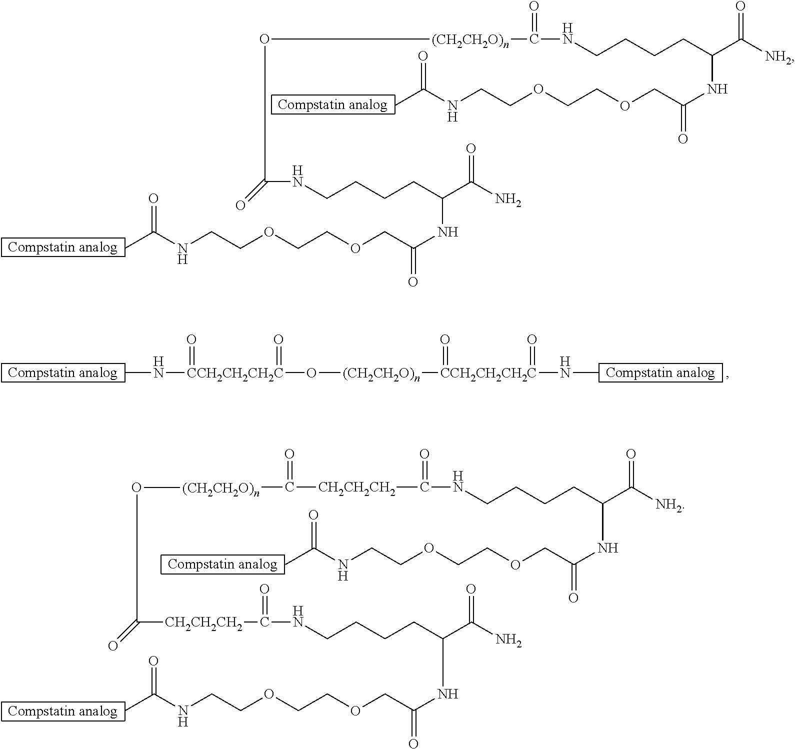

In certain embodiments a compstatin analog may be represented as M-AEEAc-Lys-B₂, wherein B₂ is a blocking moiety, e.g., NH₂, M represents any of SEQ ID NOs: 3-36, 37, 69, 70, 71, or 72, with the proviso that the C-terminal amino acid of any of SEQ ID NOs: 3-36, 37, 69, 70, 71, or 72 is linked via a peptide bond to AEEAc-Lys-B₂. The NHS moiety of a monofunctional or multifunctional (e.g., bifunctional) PEG reacts with the free amine of the lysine side chain to generate a monofunctionalized (one compstatin analog moiety) or multifunctionalized (multiple compstatin analog moieties) PEGylated compstatin analog. In various embodiments any amino acid comprising a side chain that comprises a reactive functional group may be used instead of Lys (or in addition to Lys). A monofunctional or multifunctional PEG comprising a suitable reactive functional group may be reacted with such side chain in a manner analogous to the reaction of NHS-ester activated PEGs with Lys.

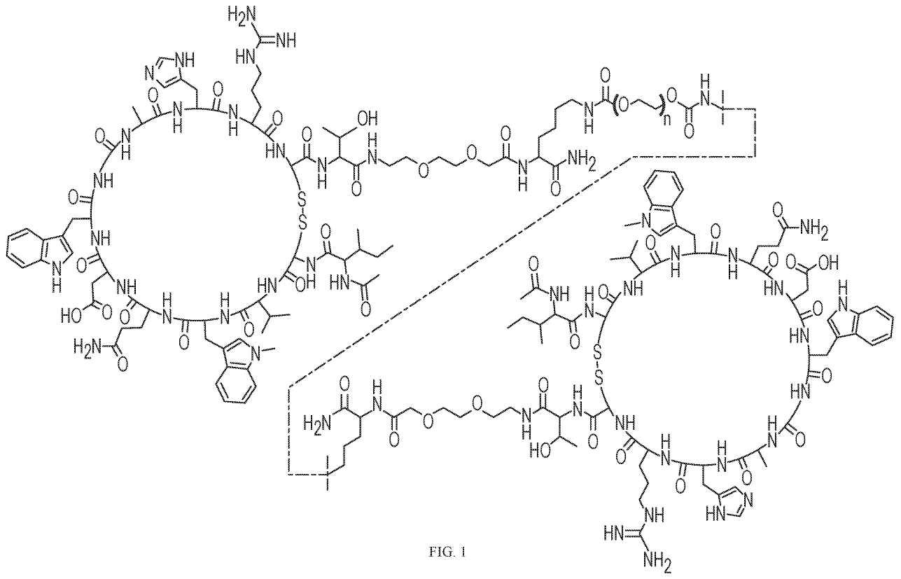

With regard to any of the above formulae and structures, it is to be understood that embodiments in which the compstatin analog component comprises any compstatin analog described herein, e.g., any compstatin analog of SEQ ID NOs; 3-36, 37, 69, 70, 71, or 72, are expressly disclosed. For example, and without limitation, a compstatin analog may comprise the amino acid sequence of SEQ ID NO: 28. An exemplary PEGylated compstatin analog in which the compstatin analog component comprises the amino acid sequence of SEQ ID NO: 28 is depicted in FIG. 1. It will be understood that the PEG moiety may have a variety of different molecular weights or average molecular weights in various embodiments, as described herein. For example, individual PEG chains within a preparation may vary in molecular weight and/or different preparations may have different average molecular weights and/or polydispersity, as described herein. In certain embodiments, “n” in the compound of FIG. 1 has a value such that the PEG moiety in the compound of FIG. 1 has an average molecular weight of about 10 kD. In certain embodiments, “n” in the compound of FIG. 1 is between about 200 and about 275.

In some aspects, the present invention relates to use of click chemistry in connection with compstatin analogs. “Click chemistry” is well known in the art and is useful in some aspects of the present invention. Click chemistry embodies, in certain embodiments, versatile cycloaddition reactions between azides and alkynes that enable a number of useful applications. Methods of carrying out click chemistry are known in the art, and are described by Kolb, H. C.; Sharpless, K. B., Drug Disc. Today, 2003, 1128-1137; Moses, J. E.; Moorhouse, A. D.; Chem. Soc. Rev., 2007, 1249-1262; the entire contents of each are hereby incorporated by reference. Click chemistry is a popular method of bioconjugation due to its high reactivity and selectivity, even in biological media. See Kolb, H. C.; Finn, M. G.; Sharpless, K. B. Angew. Chem. Int. Ed. 2001, 40, 2004-2021; and Wang, Q.; Chan, T. R.; Hilgraf, R.; Fokin, V. V.; Sharpless, K. B.; Finn, M. G. J. Am. Chem. Soc. 2003, 125, 3192-3193. In addition, currently available recombinant techniques and synthetic methods permit the introduction of azides and alkyne-bearing non-canonical amino acids into peptides, proteins, cells, viruses, bacteria, and other biological entities that consist of or display proteins. See Link, A. J.; Vink, M. K. S.; Tirrell, D. A. J. Am. Chem. Soc. 2004, 126, 10598-10602; Deiters, A.; Cropp, T. A.; Mukherji, M.; Chin, J. W.; Anderson, C.; Schultz, P. G. J. Am. Chem. Soc. 2003, 125, 11782-11783.

As used herein, the term “click chemistry group” is sometimes used to refer to a reactive functional group capable of participating in a click chemistry reaction with an appropriate second reactive functional group, which second reactive functional group is also a click chemistry group. The first and second click chemistry groups, or entities (e.g., molecules) comprising such groups, may be referred to as complementary. First and second entities, e.g., molecules, that comprise complementary click chemistry groups may be referred to as click chemistry partners. An entity or molecule comprising a click chemistry group may be referred to as “click-functionalized”. A bond formed by reaction of complementary click chemistry partners may be referred to as a “click chemistry bond”.

In some embodiments, the present invention provides click-functionalized compstatin analogs for, e.g., conjugation to a complementary moiety, e.g., a PEG. In some embodiments, the “click-functionalized” moiety is an alkyne or an alkyne derivative which is capable of undergoing [3+2] cycloaddition reactions with complementary azide-bearing molecules and biomolecules. In another embodiment, the “click-functionalized” functionality is an azide or an azide derivative which is capable of undergoing [3+2] cycloaddition reactions with complementary alkyne-bearing molecules and biomolecules (i.e. click chemistry).

In some embodiments, a click-functionalized compstatin analog bears an azide group on any side chain group of the compstatin analog. In some embodiments, a click-functionalized compstatin analog bears an azide group on a lysine side chain group.

In some embodiments, a click-functionalized compstatin analog bears an alkyne group on any side chain group of the compstatin compstatin analog. In some embodiments, a click-functionalized compstatin analog bears an alkyne group on a lysine side chain group.

In some embodiments, the present invention provides compstatin conjugates comprising a compstatin analog, a PEG moiety, and a triazole linker. In some embodiments, a triazole linker is the result of click conjugation chemistry between a compstatin conjugate and a PEG moiety.

In some embodiments, click chemistry between a compstatin analog and another moiety is transition metal catalyzed. Copper-containing molecules which catalyze the “click” reaction include, but are not limited to, copper wire, copper bromide (CuBr), copper chloride (CuCl), copper sulfate (CuSO₄), copper sulfate pentahydrate (CuSO₄•5H₂O), copper acetate (Cu₂(AcO₄), copper iodide (CuI), [Cu(MeCN)₄](OTf), [Cu(MeCN)₄](PF₆), colloidal copper sources, and immobilized copper sources. In some embodiments other metals, such as ruthenium. Reducing agents as well as organic and inorganic metal-binding ligands can be used in conjunction with metal catalysts and include, but are not limited to, sodium ascorbate, tris(triazolyl)amine ligands, tris(carboxyethyl)phosphine (TCEP), sulfonated bathophenanthroline ligands, and benzimidazole-based ligands.

In some embodiments, compstatin analogs are conjugated to other moieties using metal free click chemistry (also known as copper free click chemistry) to give a metal free composition or conjugates. In contrast to standard click chemistry, also known as copper assisted click chemistry (CuACC), metal free click chemistry occurs between either a strained, cyclic alkyne or an alkyne precursor such as an oxanorbornadiene, and an azide group. As the name implies, no metal catalyst is necessary for the reaction to occur. Examples of such chemistries include reactions involving cyclooctyne derivatives (Codelli, et. al. J. Am. Chem. Soc., 2008, 130, 11486-11493; Jewett, et. al. J. Am. Chem. Soc., 2010, 132, 3688-3690; Ning, et. al. Angew. Chem. Int. Ed., 2008, 47, 2253-2255), difluoro-oxanorbornane derivatives (van Berkel, et. al. ChemBioChem, 2007, 8, 1504-1508), or nitrile oxide derivatives (Lutz, et. al. Macromolecules, 2009, 42, 5411-5413). In certain embodiments a metal-free click chemistry reaction is a metal-free [3+2] cycloaddition reaction, Diels-Alder reaction, or thiol-alkene radical addition reaction. Exemplary click chemistry reactions and click chemistry groups are described in, e.g., Joerg Lahann, Click Chemistry for Biotechnology and Materials Science, 2009, John Wiley & Sons Ltd, ISBN 978-0-470-69970-6; Becer, Hoogenboom, and Schubert, Click Chemistry beyond Metal-Catalyzed Cycloaddition, Angewandte Chemie International Edition (2009) 48: 4900-4908. In certain embodiments a click chemistry group comprises a diarylcyclooctyne.

Certain examples of metal free click chemistry are shown in the scheme below.

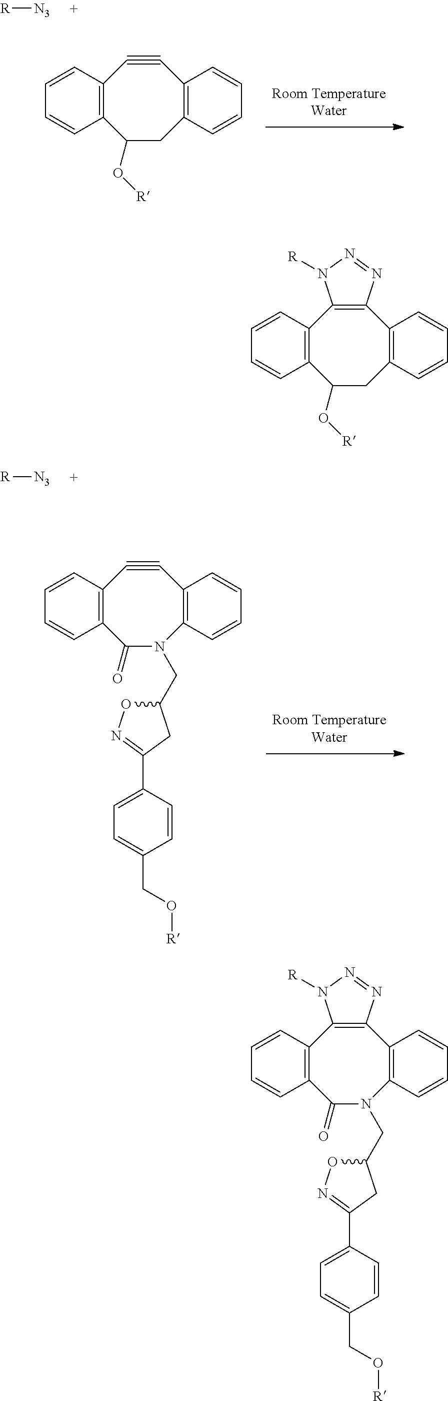

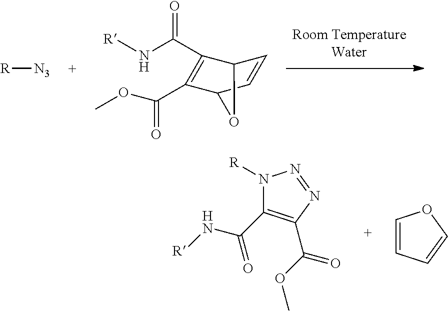

Certain metal-free click moieties are known in the literature. Examples include 4-dibenzocyclooctynol (DIBO)

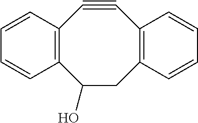 [SMILES: OC1Cc2ccccc2C#Cc2ccccc21]

(from Ning et. al, Angew Chem Int Ed, 2008, 47, 2253); difluorinated cyclooctynes (DIFO or DFO)

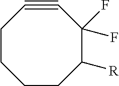

(from Codelli, et. al.; J. Am.

Chem. Soc. 2008, 130, 11486-11493); biarylazacyclooctynone (BARAC)

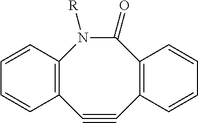

(from Jewett et. al.; J. Am. Chem. Soc. 2010, 132, 3688); or bicyclononyne (BCN)

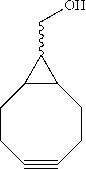 [SMILES: OCC1C2CCC#CCCC12]

(From Dommerholt, et. al.; Angew Chem Int Ed, 2010, 49, 9422-9425) or dibenzylcyclooctene (DBCO)

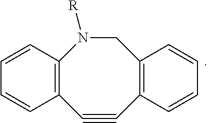

A reaction scheme involving reaction of DBCO and an azide is shown below:

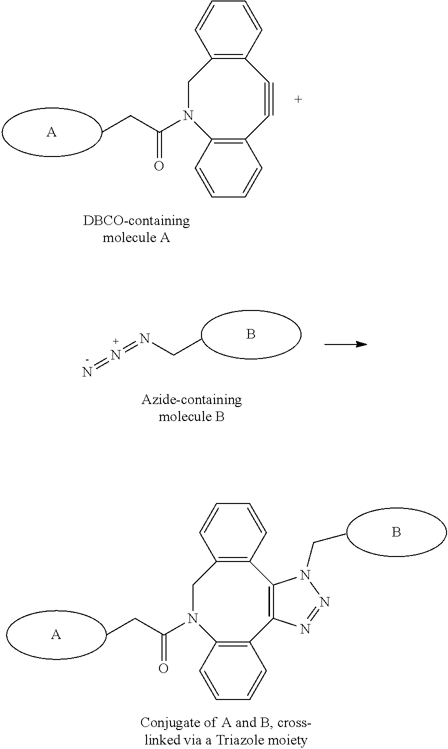

In the above scheme, in various embodiments, A may comprise or consist of a compstatin analog moiety and B may comprise or consist of a PEG, or B may comprise or consist of a compstatin analog moiety and A may comprise or consist of a PEG

In some embodiments, the “metal free click-functionalized” moiety is an acetylene or an acetylene derivative which is capable of undergoing [3+2] cycloaddition reactions with complementary azide-bearing molecules and biomolecules without the use of a metal catalyst.

In some embodiments, the R and R′ groups of the metal-free click chemistry reagents are a compstatin analog or any molecule described herein to which a compstatin analog may be conjugated. In some embodiments, such compstatin analogs bear a click-functionalized moiety on a lysine side chain. In some embodiments, such compstatin analogs are connected to a click-functionalized moiety via a linker. In some embodiments, such compstatin analogs are connected to a click-functionalized moiety via AEEAc.

In some embodiments, a click chemistry reagent comprises DBCO. Exemplary reagents and exemplary uses thereof are set forth below:

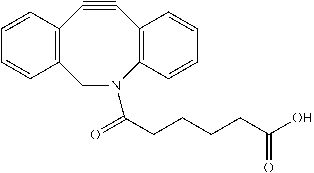 [SMILES: O=C(O)CCCCC(=O)N1Cc2ccccc2C#Cc2ccccc21]

DBCO-Acid. In some embodiments a DBCO-Acid may be used to react with an amine-containing moiety.

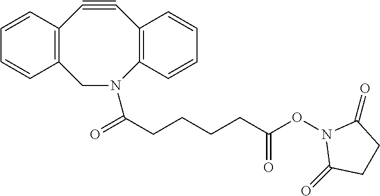 [SMILES: O=C(CCCCC(=O)N1Cc2ccccc2C#Cc2ccccc21)ON1C(=O)CCC1=O]

DBCO-NHS ester (above) or DBCO-sulfo-NHS ester (below) may be used to incorporate a DBCO functionality into an amine-containing molecule, such as a compstatin analog or a polypeptide comprising a lysine residue.

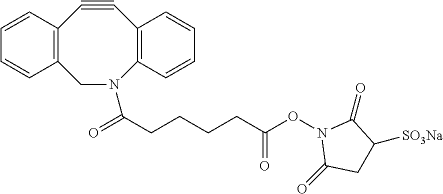

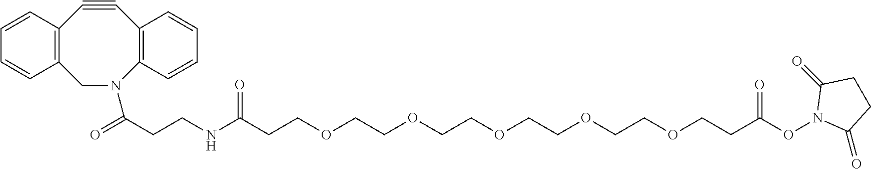 [SMILES: O=C(CCOCCOCCOCCOCCOCCC(=O)ON1C(=O)CCC1=O)NCCC(=O)N1Cc2ccccc2C#Cc2ccccc21]

DBCO-PEG4-NHS ester. In some embodiments such reagent is useful for introducing a DBCO moiety by reaction with an available amine functionality. In some aspects, the presence of a PEG chain as a hydrophilic spacer may be useful to, e.g., increase solubility or provide flexibility.

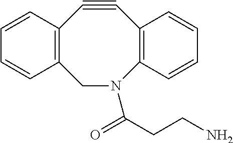 [SMILES: NCCC(=O)N1Cc2ccccc2C#Cc2ccccc21]

DBCO-Amine. In some embodiments a click chemistry reagent comprises a carbonyl/carboxyl reactive dibenzylcyclooctyne, which may react with acids, active esters and/or aldehydes.

In certain embodiments a click chemistry reaction involves a cyclooctyne depicted below.

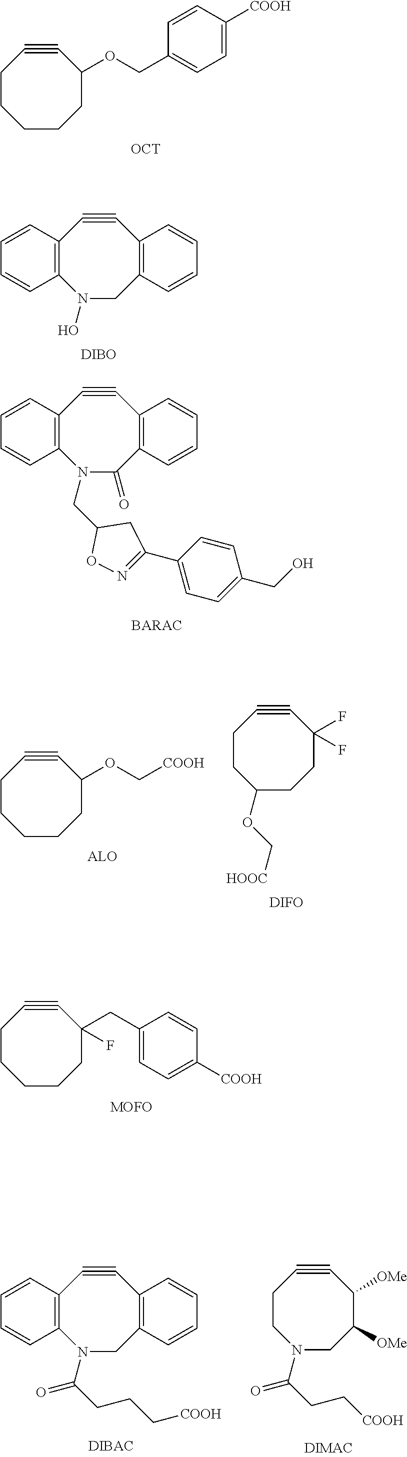

In certain embodiments click chemistry reactions comprise reactions between nitrones and cyclooctynes (see, e.g., Ning, Xinghai; Temming, Rinske P.; Dommerholt, Jan; Guo, Jun; Ania, Daniel B.; Debets, Marjoke F.; Wolfert, Margreet A.; Boons, Geert-Jan et al. (2010). “Protein Modification by Strain-Promoted Alkyne-Nitrone Cycloaddition”. Angewandte Chemie International Edition 49 (17): 3065), oxime/hydrazone formation from aldehydes and ketones, tetrazine ligations (see, e.g., Blackman, Melissa L.; Royzen, Maksim; Fox, Joseph M. (2008). “The Tetrazine Ligation: Fast Bioconjugation based on Inverse-electron-demand Diels-Alder Reactivity”. Journal of the American Chemical Society 130 (41): 13518-9), tetrazole ligations, the isonitrile-based click reaction (see, e.g., Stackmann, Henning; Neves, AndrÃ© A.; Stairs, Shaun; Brindle, Kevin M.; Leeper, Finian J. (2011). “Exploring isonitrile-based click chemistry for ligation with biomolecules”. Organic & Biomolecular Chemistry 9 (21): 7303), and the quadricyclane ligation (see, e.g., Sletten, Ellen M.; Bertozzi, Carolyn R. (2011). “A Bioorthogonal Quadricyclane Ligation”. Journal of the American Chemical Society 133 (44): 17570-3). In certain embodiments a click chemistry reaction is a Staudinger ligation (phosphine-azide).

Any compstatin analog may be modified to incorporate a click chemistry group in various embodiments. For example, a compstatin analog comprising the sequence of any of SEQ ID NOs: 3-36, 37, 69, 70, 71, or 72 may be so modified. In some embodiments any such sequence further comprises a lysine residue or an AEEAc-Lys moiety, e.g., at the C-terminus. In some embodiments a click chemistry group is incorporated after peptide synthesis. For example, a Lys side chain may be reacted with azido acetic acid in order to introduce an azide moiety as a click chemistry group. In some embodiments a click chemistry group is incorporated after cyclization and, in some embodiments, after addition of a blocking moiety at the N- and/or C-terminus. In some embodiments a click chemistry group is incorporated during peptide synthesis. For example, an amino acid comprising a side chain that comprises a click chemistry group may be used in the synthesis of a compstatin analog. A variety of such amino acids are commercially available from a number of sources, e.g., AAPPTec (Louisville, Ky.), Jena Bioscience GmBH (Jena, Germany). In some aspects, methods of making a click chemistry functionalized compstatin analog are provided herein.

In some embodiments compositions comprising a compstatin analog and a click chemistry reagent are provided. The click chemistry reagent may be any molecule capable of reacting with an amino acid side chain or terminus of a compound comprising a compstatin analog so as to install a click chemistry group, e.g., any click chemistry group known in the art. In some aspects, the composition may be incubated under suitable conditions (which may include providing a suitable catalyst, light (e.g., UV)) to functionalize the compstatin analog with a click chemistry functionality. In some embodiments, the invention provides compstatin analogs that comprise any click chemistry group including, but not limited to, those described herein. In some embodiments methods of making a PEGylated compstatin analog are provided. In some embodiments the methods comprise mixing a compstatin analog comprising a first click chemistry group with a PEG comprising a complementary click chemistry group under conditions suitable for a click chemistry reaction to occur. Additional steps may comprise purifying the resulting conjugate. In some embodiments purifying comprises removing at least some unreacted components, e.g., with an appropriate scavenger.

In some embodiments a click chemistry reaction is used to join two or more PEGs, at least two of which have a compstatin analog moiety attached thereto. The compstatin analog moieties may be the same or different in various embodiments. The compstatin analog moieties may or may not be attached to the PEG via a click chemistry reaction. For example, in some embodiments a first heterobifunctional PEG comprising a first click chemistry group at a first terminus and an NHS ester at a second terminus is coupled to a compstatin analog moiety via the NHS ester. In a separate reaction, a second heterobifunctional PEG comprising a second click chemistry group at a first terminus and an NHS ester at a second terminus is coupled to a compstatin analog moiety via the NHS ester. The resulting two compounds are then reacted via a click chemistry reaction to form a larger molecule comprising two compstatin analog moieties.

Compstatin analogs comprising a click chemistry group have a variety of uses. In some embodiments a compstatin analog comprising a first click chemistry group is reacted with any entity that comprises a complementary click chemistry group. The entity comprising the complementary click chemistry group may comprise, for example, a label (e.g., a fluorophore, fluorescent protein, radioisotope, etc.), an affinity reagent, an antibody, a targeting moiety, a metal, a particle, etc. In some embodiments a click chemistry group is used to attach a compstatin analog moiety to a surface, wherein the surface comprises or is functionalized to comprise a complementary click chemistry group. In some embodiments a surface is for a sensor, e.g., a surface or sensor for capture/detection of C3. In some embodiments a surface forms part of a medical device, tubing, membrane, reservoir, implant, or other material that may come in contact with blood (e.g., extracorporeally) or be temporarily or indefinitely implanted into the body of a subject (e.g., a prosthetic device or drug delivery device). In some embodiments a surface is functionalized with compstatin analog to reduce complement activation thereon. In some embodiments a device or tubing is used for circulating blood, e.g., for dialysis, during surgery, etc. In some embodiments a device is a hemodialyzer or an extracorporeal circulatory support unit. Such compstatin analog functionalized devices and methods of making thereof are provided herein.

In some aspects, a PEGylated compstatin analog comprises a compound of formula A-L-M, wherein A is a PEG, L is an optionally present linking portion, and M comprises a compstatin analog moiety. The compstatin analog moiety can comprise any compstatin analog, e.g., any compstatin analog described above, in various embodiments. Formula A-L-M encompasses embodiments in which A-L is present at the N-terminus of the compstatin analog moiety, embodiments in which A-L is present at the C-terminus of the compstatin analog moiety, embodiments in which A-L is attached to a side chain of an amino acid of the compstatin analog moiety, and embodiments where the same or different A-Ls are present at both ends of M. It will be appreciated that when certain compstatin analog(s) are present as a compstatin analog moiety in a compound of formula A-L-M, a functional group of the compstatin analog will have reacted with a functional group of L to form a covalent bond to A or L. For example, a PEGylated compstatin analog in which the compstatin analog moiety comprises a compstatin analog that contains an amino acid with a side chain containing a primary amine (N12) group (which compstatin analog can be represented by formula R¹—(NH₂)), can have a formula R¹—NH-L-A in which a new covalent bond to L (e.g., N—C) has been formed and a hydrogen lost. Thus the term “compstatin analog moiety” includes molecular structures in which at least one atom of a compstatin analog participates in a covalent bond with a second moiety, which may, e.g., modification of a side chain. Similar considerations apply to compstatin analog moieties present in multivalent compounds described herein. In some embodiments, a blocking moiety at the N-terminus or C-terminus of a compstatin analog, e.g., a compstatin analog described herein, is replaced by A-L in the structure of a PEGylated compstatin analog. In some embodiments, A or L comprises a blocking moiety. In some embodiments, a PEGylated compstatin analog has a molar activity of at least about 10%, 20%, or 30%, e.g., between 30% and 40%, between 30% and 50%, between 30% and 60%, between 30% and 70%, between 30% and 80%, between 30% and 90%, or more, of the activity of a corresponding compstatin analog having the same amino acid sequence (and, if applicable, one or more blocking moiet(ies)) but not comprising a PEG. In some embodiments in which a PEGylated compstatin analog comprises multiple compstatin analog moieties, the molar activity of the PEGylated compstatin analog is at least about 10%, 20%, or 30%, e.g., between 30% and 40%, between 30% and 50%, between 30% and 60%, between 30% and 70%, between 30% and 80%, between 30% and 90%, or more, of the sum of the activities of said compstatin analog moieties.

In general, linking portion L can comprise any one or more aliphatic and/or aromatic moieties consistent with the formation of a stable compound joining the linked moieties. The term “stable”, as used herein, preferably refers to compounds which possess stability sufficient to allow manufacture and which maintain the integrity of the compound for a sufficient period of time, e.g., to be useful for one or more purposes described herein. In some embodiments, L comprises a saturated or unsaturated, substituted or unsubstituted, branched or unbranched, aliphatic chain having a length of between 1 and 30, between 1 and 20, between 1 and 10, between 1 and 6, or 5 or less carbon atoms, where length refers to the number of C atoms in the main (longest) chain. In some embodiments, the aliphatic chain comprises one or more heteroatoms (O, N, S), which may be independently selected. In some embodiments, at least 50% of the atoms in the main chain of L are carbon atoms. In some embodiments, L comprises a saturated alkyl moiety (CH₂)ₙ, wherein n is between 1 and 30.

In some embodiments, L comprises one or more heteroatoms and has a length of between 1 and 1000, between 1 and 800, between 1 and 600, between 1 and 400, between 1 and 300, between 1 and 200, between 1 and 100, between 1 and 50, between 1 and 30, or between 1 and 10 total carbon atoms in a chain. In some embodiments, L comprises an oligo(ethylene glycol) moiety (—(O—CH₂—CH₂-)ₙ) wherein n is between 1 and 500, between 1 and 400, between 1 and 300, between 1 and 200, between 1 and 100, between 10 and 200, between 200 and 300, between 100 and 200, between 40 and 500, between 30 and 500, between 20 and 500, between 10 and 500, between 1 and 40, between 1 and 30, between 1 and 20, or between 1 and 10.

In some embodiments, L comprises an unsaturated moiety such as —CH═CH— or —CH₂—CH═CH—; a moiety comprising a non-aromatic cyclic ring system (e.g., a cyclohexyl moiety), an aromatic moiety (e.g., an aromatic cyclic ring system such as a phenyl moiety); an ether moiety (—C—O—C—); an amide moiety (—C(═O)—N—); an ester moiety (—CO—O—); a carbonyl moiety (—C(═O)—); an imine moiety (—C═N—); a thioether moiety (—C—S—C—); an amino acid residue; and/or any moiety that can be formed by the reaction of two compatible reactive functional groups. In certain embodiments, one or more moieties of a linking portion or cell-reactive moiety is/are substituted by independent replacement of one or more of the hydrogen (or other) atoms thereon with one or more moieties including, but not limited to aliphatic; aromatic, aryl; alkyl, aralkyl, alkanoyl, aroyl, alkoxy; thio; F; C1; Br; I; —NO2; —CN; —CF3; —CH2CF3; —CHC12; —CH2OH; —CH2CH2OH; —CH2NH2; —CH2SO2CH3; - or -GRG1 wherein G is —O—, —S—, —NRG2-, —C(═O)—, —S(═O)—, —SO2-, —C(═O)O—, —C(═O)NRG2-, —OC(═O)—, —NRG2C(═O)—, —OC(═O)O—, —OC(═O)NRG2-, —NRG2C(═O)O—, —NRG2C(═O)NRG2-, —C(═S)—, —C(═S)S—, —SC(═S)—, —SC(═S)S—, —C(═NRG2)-, —C(═NRG2)O—, —C(═NRG2)NRG3-, —OC(═NRG2)-, —NRG2C(═NRG3)-, —NRG2SO2-, —NRG2SO2NRG3-, or —SO₂NRG2-, wherein each occurrence of RG1, RG2 and RG3 independently includes, but is not limited to, hydrogen, halogen, or an optionally substituted aliphatic, aromatic, or aryl moiety. It will be appreciated that cyclic ring systems when present as substituents may optionally be attached via a linear moiety. Combinations of substituents and variables envisioned by this invention are preferably those that result in the formation of stable compounds useful in any one or more of the methods described herein, e.g., useful for the treatment of one or more disorders and/or for contacting a cell, tissue, or organ, as described herein, and/or useful as intermediates in the manufacture of one or more such compounds.

L can comprise one or more of any of the moieties described in the preceding paragraph, in various embodiments. In some embodiments, L comprises two or more different moieties linked to one another to form a structure typically having a length of between 1 to about 60 atoms, between 1 to about 50 atoms, e.g., between 1 and 40, between 1 and 30, between 1 and 20, between 1 and 10, or between 1 and 6 atoms, where length refers to the number of atoms in the main (longest) chain. In some embodiments, L comprises two or more different moieties linked to one another to form a structure typically having between 1 to about 40, e.g., between 1 and 30, e.g., between 1 and 20, between 1 and 10, or between 1 and 6 carbon atoms in the main (longest) chain. In general, the structure of such a PEGylated compstatin analog can be represented by formula A-(L^{Pj})j-M, wherein j is typically between 1 and 10, and each L^{Pj} is independently selected from among the moieties described in the preceding paragraph. In many embodiments, L comprises one or more carbon-containing chains such as —(CH₂)ₙ- and/or —(O—CH₂—CH₂-)ₙ, which are joined covalently to each other and/or to a cell-reactive functional group or compstatin analog, e.g., by moieties (e.g., amide, ester, or ether moieties) that result from the reaction of two compatible reactive functional groups. In some embodiments, L comprises an oligo(ethylene glycol) moiety and/or a saturated alkyl chain. In some embodiments, L comprises —(CH₂)ₘ—C(═O)—NH—(CH₂CH₂O)ₙ(CH₂)ₚC(═O)— or —(CH₂)ₘ—C(═O)—NH—(CH₂)ₚ(OCH₂CH₂)ₙC(═O)—. In some embodiments, m, n, and p are selected so that the number of carbons in the chain is between 1 and 500, e.g., between 2 and 400, between 2 and 300, between 2 and 200, between 2 and 100, between 2 and 50, between 4 and 40, between 6 and 30, or between 8 and 20. In some embodiments, m is between 2 and 10, n is between 1 and 500, and/or p is between 2 and 10. In some embodiments, m is between 2 and 10, n is between 1 and 400, and/or p is between 2 and 10. In some embodiments, m is between 2 and 10, n is between 1 and 300, and/or p is between 2 and 10. In some embodiments, m is between 2 and 10, n is between 1 and 200, and/or p is between 2 and 10. In some embodiments, m is between 2 and 10, n is between 1 and 100, and/or p is between 2 and 10. In some embodiments, m is between 2 and 10, n is between 1 and 50, and/or p is between 2 and 10. In some embodiments, m is between 2 and 10, n is between 1 and 25, and/or p is between 2 and 10. In some embodiments, m is between 2 and 10, n is between 1 and 8, and/or p is between 2 and 10. Optionally, at least one —CH₂- is replaced by CH—R, wherein R can be any substituent. Optionally, at least one —CH₂- is replaced by a heteroatom, cyclic ring system, amide, ester, or ether moiety. In some embodiments, L does not comprise an alkyl group having more than 3 carbon atoms in the longest chain. In some embodiments, L does not comprise an alkyl group having more than 4, 5, 6, 7, 8, 9, 10, or 11 carbon atoms in the longest chain.

In some embodiments of the invention, A comprises a PEG moiety described herein and a linker L¹ comprising a linking portion L^{P1} and a reactive functional group that reacts with the compstatin analog to generate A-M In some embodiments, a bifunctional linker L² comprising two reactive functional groups and a linking portion L^{P2} is used. The reactive functional groups of L react with appropriate reactive functional groups of A and M to produce a PEGylated compstatin analog A-L-M. In some embodiments, the compstatin analog comprises a linker L³ comprising a linking portion L^{P3}. For example, as discussed herein, a linker comprising a reactive functional group may be present at the N- or C-terminus or a moiety comprising a reactive functional group may be attached to the N- or C-terminus via a linker. Thus L may contain multiple linking portions LP contributed, e.g., by A, by linker(s) used to join A and M, and/or by the compstatin analog. It will be understood that, when present in the structure A-L-M, certain reactive functional group(s) present prior to reaction in L¹, L², L³, etc., will have undergone reaction, so that only a portion of said reactive functional group(s) will be present in the final structure A-L-M, and the compound will contain moieties formed by reaction of said functional groups. In general, if a compound contains two or more linking portions, the linking portions can be the same or different, and can be independently selected in various embodiments. Multiple linking portions LP can be attached to one another to form a larger linking portion L, and at least some of such linking portions can have one or more compstatin analog(s) and/or PEGs attached thereto. In molecules comprising multiple compstatin analogs, the compstatin analogs can be the same or different and, if different, can be independently selected. The same applies to the linking portions and reactive functional groups.

In some embodiments a PEGylated compstatin analog comprises a compstatin analog in which any of SEQ ID NOs: 3-36, 69, 70, 71, or 72 is extended by one or more amino acids at the N-terminus, C-terminus, or both, wherein at least one of the amino acids has a side chain that comprises a reactive functional group such as a primary or secondary amine, a sulfhydryl group, a carboxyl group (which may be present as a carboxylate group), a guanidino group, a phenol group, an indole ring, a thioether, or an imidazole ring. In some embodiments, the amino acid(s) is/are L-amino acids. In some embodiments, any one or more of the amino acid(s) is a D-amino acid. If multiple amino acids are added, the amino acids can be independently selected. In some embodiments, the reactive functional group (e.g., a primary or secondary amine) is used as a target for addition of a PEG moiety. Amino acids having a side chain that comprises a primary or secondary amine include lysine (Lys) and diaminocarboxylic acids of general structure NH₂(CH₂)ₙCH(NH₂)COOH such as 2,3-diaminopropionic acid (dapa), 2,4-diaminobutyric acid (daba), and ornithine (orn), wherein n=1 (dapa), 2 (daba), and 3 (orn), respectively. In some embodiments at least one amino acid is cysteine, aspartic acid, glutamic acid, arginine, tyrosine, tryptophan, methionine, or histidine. Cysteine has a side chain comprising a sulfhydryl group. Aspartic acid and glutamic acid have a side chain comprising a carboxyl group (ionizable to a carboxylate group). Arginine has a side chain comprising a guanidino group. Tyrosine has a side chain comprising a phenol group (ionizable to a phenolate group). Tryptophan has a side chain comprising an indole ring include, e.g., tryptophan. Methionine has a side chain comprising a thioether group include, e.g., methionine. Histidine has a side chain comprising an imidazole ring. A wide variety of non-standard amino acids having side chains that comprise one or more such reactive functional group(s) are available, including naturally occurring amino acids and amino acids not found in nature. See, e.g., Hughes, B. (ed.), Amino Acids, Peptides and Proteins in Organic Chemistry, Volumes 1-4, Wiley-VCH (2009-2011); Blaskovich, M., Handbook on Syntheses of Amino Acids General Routes to Amino Acids, Oxford University Press, 2010. The invention encompasses embodiments in which one or more non-standard amino acid(s) is/are used to provide a target for addition of a PEG moiety. Any one or more of the amino acid(s) may be protected as appropriate during synthesis of the compound. For example, one or more amino acid(s) may be protected during reaction(s) involving the target amino acid side chain. In some embodiments, wherein a sulfhydryl-containing amino acid is used as a target for addition of a PEG moiety, the sulfhydryl is protected while the compound is being cyclized by formation of an intramolecular disulfide bond between other amino acids such as cysteines.

In the discussion in this paragraph, an amino acid having a side chain containing an amine group is used as an example. The invention encompasses analogous embodiments in which an amino acid having a side chain containing a different reactive functional group is used. In some embodiments, an amino acid having a side chain comprising a primary or secondary amine is attached directly to the N-terminus or C-terminus of any of SEQ ID NOs: 3-36, 37, 69, 70, 71, or 72 or via a peptide bond. In some embodiments, an amino acid having a side chain comprising a primary or secondary amine is attached to the N- or C-terminus of any of SEQ ID NOs: 3-36, 37, 69, 70, 71, or 72, or via a linking portion, which may contain any one or more of the linking moieties described above. In some embodiments, at least two amino acids are appended to either or both termini. The two or more appended amino acids may be joined to each other by peptide bonds or at least some of the appended amino acids may be joined to each other by a linking portion, which may contain any one or more of the linking moieties described herein. Thus in some embodiments, a PEGylated compstatin analog comprises a compstatin analog moiety M of formula B1-R1-M1-R2-B2, wherein M1 represents any of SEQ ID NOs: 3-36, 37, 69, 70, 71, or 72, either R1 or R2 may be absent, at least one of R1 and R2 comprises an amino acid having a side chain that contains a primary or secondary amine, and B1 and B2 are optionally present blocking moieties. R1 and/or R2 may be joined to M₁ by a peptide bond or a non-peptide bond. R1 and/or R2 may comprise a linking portion L^{P3}. For example, R1 can have formula M2-L^{P3} and/or R2 can have formula L^{P3}-M2 wherein L^{P3} is a linking portion, and M2 comprises at least one amino acid having a side chain comprising a primary or secondary amine. For example, M2 can be Lys or an amino acid chain comprising Lys. In some embodiments, L^{P3} comprises of consists of one or more amino acids. For example, L^{P3} can be between 1 and about 20 amino acids in length, e.g., between 4 and 20 amino acids in length. In some embodiments, L^{P3} comprises or consist of multiple Gly, Ser, and/or Ala residues. In some embodiments, L^{P3} does not comprise an amino acid that comprises a reactive SH group, such as Cys. In some embodiments, L^{P3} comprises an oligo(ethylene glycol) moiety and/or a saturated alkyl chain. In some embodiments, L^{P3} is attached to the N-terminal amino acid of M1 via an amide bond. In some embodiments, L^{P3} is attached to the C-terminal amino acid of M1 via an amide bond. The compound may be further extended at either or both termini by addition of further linking portion(s) and/or amino acid(s). The amino acids can the same or different and, if different, can be independently selected. In some embodiments, two or more amino acids having side chains comprising reactive functional groups are used, wherein the reactive functional groups can be the same or different. The two or more reactive functional groups can be used as targets for addition of two or more PEG moieties. In some embodiments, a linker and/or PEG moiety is attached to an amino acid side chain after incorporation of the amino acid into a peptide chain. In some embodiments, a linker and/or PEG moiety is already attached to the amino acid side chain prior to use of the amino acid in the synthesis of a PEGylated compstatin analog. For example, a Lys derivative having a linker attached to its side chain can be used. The linker may comprise a PEG moiety or may subsequently be modified to comprise a PEG moiety.

In some embodiments, an amino acid having a linker attached to a side chain is used in the synthesis of a linear peptide. The linear peptide can be synthesized using standard methods for peptide synthesis known in the art, e.g., standard solid-phase peptide synthesis. The linear peptide is then cyclized (e.g., by oxidation of the Cys residues to form an intramolecular disulfide). The cyclic compound may then be reacted with a linker comprising a PEG moiety. In other embodiments, a PEG moiety is reacted with a linear compound prior to cyclization thereof. In general, reactive functional groups can be appropriately protected to avoid undesired reaction with each other during synthesis of a PEGylated compstatin analog. The PEG moiety, any of the amino acid side chains, and/or either or both termini of the peptide may be protected during the reaction and subsequently deprotected. The reaction conditions are selected based at least in part on the requirements of the particular reactive functional group(s) to achieve reasonable yield in a reasonable time period. Temperature, pH, and the concentration of the reagents can be adjusted to achieve the desired extent or rate of reaction. See, e.g., Hermanson, supra. The desired product can be purified, e.g., to remove unreacted compound comprising the PEG moiety, unreacted compstatin analog, linker(s), products other than the desired PEGylated compstatin analog that may have been generated in the reaction, other substances present in the reaction mixture, etc. Compositions and methods for making the PEGylated compstatin analogs, and intermediates in the synthesis, are aspects of the invention.

Compstatin analog moieties may comprise a peptide whose sequence comprises any of SEQ ID NOs: 3-36, 37, 69, 70, 71, or 72, or variants thereof (e.g., any variant described herein), optionally extended by one or more amino acids at the N-terminus, C-terminus, or both wherein at least one of the amino acids has a side chain that comprises a reactive functional group such as a primary or secondary amine (e.g., a Lys), a sulfhydryl group, a carboxyl group (which may be present as a carboxylate group), a guanidino group, a phenol group, an indole ring, a thioether, or an imidazole ring, which facilitates conjugation of a PEG moiety to the compstatin analog (it being understood that after conjugation, such reactive functional group will have reacted to form a bond). It will further be understood that where a compstatin analog moiety comprising any of SEQ ID NOs: 3-36, 37, 69, 70, 71, or 72, or variants thereof, is extended by one or more amino acids at the N-terminus, C-terminus, or both wherein at least one of the amino acids has a side chain that comprises a reactive functional group, such one or more amino acid extension may be separated from the cyclic portion of the compstatin analog moiety by a rigid or flexible spacer moiety comprising, for example, a substituted or unsubstituted, saturated or unsaturated alkyl chain, oligo(ethylene glycol) chain, and/or any of the other moieties denoted by L (or L^{P1}, L^{P2}, or L^{P3}) herein.

Exemplary PEGylated compstatin analogs are set forth below, wherein n is sufficient to provide an average molecular weight of about 500; 1,000; 1,500; 2,000; 5,000; 10,000; 15,000; or 20,000 daltons. In some embodiments n is sufficient to provide an average molecular weight of about 10,000 daltons.

(CH₂CH₂O)ₙC(═O)—Ile-Cys-Val-(1Me)Trp-Gln-Asp-Trp-Gly-Ala-His-Arg-Cys-Thr-NH₂) (SEQ ID NO: 58)

Ac-Ile-Cys*-Val-(1Me)Trp-Gln-Asp-Trp-Gly-Ala-His-Arg-Cys*-Thr —NH—CH₂CH₂OCH₂CH₂OCH₂—C(═O)-Lys-C(═O)—(CH₂CH₂O)ₙ—NH₂ (SEQ ID NO: 59)

Ac-Ile-Cys*-Val-(1Me)Trp-Gln-Asp-Trp-Gly-Ala-His-Arg-Cys*-Thr-Lys-C(═O)—(CH₂CH₂O)ₙ—NH₂ (SEQ ID NO: 60).

Ac-Ile-Cys*-Val-(1Me)Trp-Gln-Asp-Trp-Gly-Ala-His-Arg-Cys*-Thr-(Gly)s-Lys-C(═O)—(CH₂CH₂O)ₙ—NH₂ (SEQ ID NO: 61)

Ac—(CH₂CH₂O)ₙC(═O)Lys-(Gly)5-Ile-Cys*-Val-(1Me)Trp-Gln-Asp-Trp-Gly-Ala-His-Arg-Cys*-Thr-NH₂) (SEQ ID NO: 62)

Ac—(CH₂CH₂O)ₙC(═O)Lys-Ile-Cys*-Val-(1Me)Trp-Gln-Asp-Trp-Gly-Ala-His-Arg-Cys*-Thr-NH₂) (SEQ ID NO: 63)

In SEQ ID NO: 58, the (CH₂CH₂O)ₙ is coupled via an amide bond to the N-terminal amino acid. In SEQ ID NOs: 59-63, the (CH₂CH₂O)ₙ moiety is coupled via an amide bond to a Lys side chain; thus it will be understood that the NH₂ at the C-terminus in SEQ ID NOs: 59, 60, and 61, represents amidation of the C-terminus of the peptide, and it will be understood that in SEQ ID NOs: 62 and 63, the Ac at the N-terminus represents acetylation of the N-terminus of the peptide, as described above. It will also be appreciated by those of ordinary skill in the art that a free end of a (CH₂CH₂O)ₙ moiety typically terminates with an (OR) where the underlined O represents the O atom in the terminal (CH₂CH₂O) group. (OR) is often a moiety such as a hydroxyl (OH) or methoxy (—OCH₃) group though other groups (e.g., other alkoxy groups) could be used. Thus SEQ ID NO: 59, for example, may be represented as Ac-Ile-Cys*-Val-(1Me)Trp-Gln-Asp-Trp-Gly-Ala-His-Arg-Cys*-Thr-NH—CH₂CH₂OCH₂CH₂CH₂—C(═O)-Lys-(C(═O)—(CH₂CH₂O)ₙ—R)—NH2 (SEQ ID NO: 64) wherein R is, e.g., either H or CH₃ in the case of a linear PEG. In the case of a bifunctional, branched or star-shaped PEG, R represents the remainder of the molecule. Further, it will be understood that the moiety comprising the reactive functional group may vary, as described herein (e.g., according to any of the formulas described herein). For example, PEGylated compstatin analogs comprising the same peptide sequence as SEQ ID NO: 64, in which the moiety comprising the reactive functional group comprises an ester and/or alkyl chain may be represented as follows

Ac-Ile-Cys*-Val-(1Me)Trp-Gln-Asp-Trp-Gly-Ala-His-Arg-Cys*-Thr-NH—CH₂CH₂OCH₂CH₂OCH₂—C(═O)-Lys-(C(═O)—(CH₂)ₘ—(CH₂CH₂O)ₙ—R)—NH₂ (SEQ ID NO: 65);
Ac-Ile-Cys*-Val-(1Me)Trp-Gln-Asp-Trp-Gly-Ala-His-Arg-Cys*-Thr-NH—CH₂CH₂OCH₂CH₂OCH₂—C(═O)-Lys-(C(═O)—(CH₂)ₘ—C(═O)—(CH₂CH₂O)ₙ—R)—NH₂ (SEQ ID NO: 66)
Ac-Ile-Cys*-Val-(1Me)Trp-Gln-Asp-Trp-Gly-Ala-His-Arg-Cys*-Thr-NH—CH₂CH₂OCH₂CH₂CH₂—C(═O)-Lys-(C(═O)—(CH₂)ₘ—C(═O)—(CH₂)j (CH₂CH₂O)ₙ—R)—NH₂ (SEQ ID NO: 67)
In SEQ ID NOs: 65-67, m may range from 1 up to about 2, 3, 4, 5, 6, 7, 8, 10, 15, 20, or 30 in various embodiments, In SEQ ID NOs: 67 j may range from 1 up to about 2, 3, 4, 5, 6, 7, 8, 10, 15, 20, or 30 in various embodiments.
It will also be appreciated that, as described herein, in various embodiments other moieties may be incorporated between the Lys-(C(═O)— and (CH₂CH₂O)ₙ—R, such as an amide, aromatic ring (e.g., a substituted or unsubstituted phenyl), or a substituted or unsubstituted cycloalkyl structure.

The invention provides variants of SEQ ID NOs: 58-67 in which -Ile-Cys*-Val-(1Me)Trp-Gln-Asp-Trp-Gly-Ala-His-Arg-Cys*-Thr- is replaced by an amino acid sequence comprising the amino acid sequence of any other compstatin analog, e.g., of any of SEQ ID NOs 3-27 or 29-36, 37, 69, 70, 71, or 72 with the proviso that blocking moiet(ies) present at the N- and/or C-termini of a compstatin analog may be absent, replaced by a linker (which may comprise a blocking moiety), or attached to a different N- or C-terminal amino acid present in the corresponding variant(s).

Any compstatin analog, e.g., any compound comprising any of SEQ ID NOs: 3-37, 69, 70, 71, or 72 may, in various embodiments, can be attached via or near its N-terminal or C-terminal end (e.g., via a side chain of an amino acid at or near its N-terminal or C-terminal amino acid) directly or indirectly to any moiety comprising a reactive functional group, e.g., any compound of Formulae I—XVI or Formulae A-H.

In some embodiments, one or more amino acids in a polypeptide or linker or composition may be selected to be hydrophobic or hydrophilic or selected to confer increased hydrophilicity or, in some embodiments, increased hydrophobicity, on a compound that contains it. As known in the art, the terms “hydrophilic” and “hydrophobic” are used to refer to the degree of affinity that a substance has with water. In some aspects a hydrophilic substance has a strong affinity for water, tending to dissolve in, mix with, or be wetted by water, while a hydrophobic substance substantially lacks affinity for water, tending to repel and not absorb water and tending not to dissolve in or mix with or be wetted by water. Amino acids can be classified based on their hydrophobicity as well known in the art. Examples of “hydrophilic amino acids” are arginine, lysine, threonine, alanine, asparagine, glutamine, aspartate, glutamate, serine, and glycine. Examples of “hydrophobic amino acids” are tryptophan, tyrosine, phenylalanine, methionine, leucine, isoleucine, and valine. In certain embodiments an analog of a standard amino acid is used, wherein the analog has increased or decreased hydrophilic or hydrophobic character as compared with the amino acid of which it is an analog.

The invention further provides multimers, e.g., concatamers, comprising two or more (e.g., between 2 and 10) compstatin analogs comprising a PEG, wherein the average molecular weight of the resulting molecule (or the PEG components thereof) is about 5,000; 10,000; 15,000; or 20,000. In some embodiments the average molecular weight of the resulting molecule (or the PEG components thereof) is about 10,000 daltons. In some embodiments, the compstatin analogs comprising a PEG can be linked using any of the linking moieties described above. Compositions and methods for making PEGylated compstatin analogs, and intermediates in the synthesis, are aspects of the invention.

In some embodiments the total molecular weight of a PEGyated compstatin analog, including the compstatin analog moieties, is no greater than 20 kD. For example, in the case of a PEGylated compstatin analog comprising a 10 kD PEG, in some embodiments the molecular weight contributed by the remainder of the compound, including the compstatin analog moie(ties), may be no greater than 10 kD, e.g., 1.5 kD-5.0 kD or 5.0 kD-10 kD. Thus, wherever the present disclosure refers to a compstatin analog comprising a PEG having a particular molecular weight, or having a molecular weight within a particular range, in some embodiments the total molecular weight of the compstatin analog may be, e.g., between 1.5 kD and 5 kD greater than the molecular weight of the PEG, or in some embodiments between 5 kD and 10 kD greater than the molecular weight of the PEG. It will be understood that molecular weight of a compound, e.g., a compound comprising a PEG, can refer to the average molecular weight of molecules of such compound in a composition.

A wide variety of methods and assays useful for detection of PEGs and/or useful for measurement of physical and/or structural properties of PEGs are known in the art and may, if desired, be used to detect a compstatin analog, e.g., a PEGylated compstatin analog or a compstatin analog moiety. For example, methods and assays useful for determining properties such as aggregation, solubility, size, structure, melting properties, purity, presence of degradation products or contaminants, water content, hydrodynamic radius, etc., are available. Such methods include, e.g., analytical centrifugation, various types of chromatography such as liquid chromatography (e.g., HPLC-ion exchange, HPLC-size exclusion, HPLC-reverse phase), light scattering, capillary electrophoresis, circular dichroism, isothermal calorimetry, differential scanning calorimetry, fluorescence, infrared (IR), nuclear magnetic resonance (NMR), Raman spectroscopy, refractometry, UV/Visible spectroscopy, mass spectrometry, immunological methods, etc. It will be understood that methods may be combined. In some aspects, a PEGylated compstatin analog (or composition comprising a PEGylated compstatin analog) has one or more properties described herein, as assessed using any of the foregoing methods.

III. Treatment Methods

In some embodiments, a compstatin analog described herein, e.g., a PEGylated compstatin analog described herein, is used to treat a subject. In some embodiments, a subject is in need of relatively short-term complement inhibition and/or in need of complement inhibition over a defined period of time followed by a relatively quick recovery from complement inhibition upon cessation of administration of such PEGylated compstatin analog.

In some embodiments, a PEGylated compstatin analog is administered to a subject in need thereof at about 10 mg to about 2500 mg (e.g., about 10 mg to about 600 mg, about 600 mg to about 1200 mg, about 1250 mg to about 2000 mg, about 2000 mg to about 2500 mg, about 10-20 mg, about 20-40 mg, about 40-60 mg, about 60-80 mg, about 80-100 mg, about 100-120 mg, about 120-140 mg, about 140-160 mg, about 160-180 mg, about 180-200 mg, about 200-220 mg, about 220-240 mg, about 240-260 mg, about 260-280 mg, about 280-300 mg, about 300-320 mg, about 320-340 mg, about 340-360 mg, about 360-380 mg, about 380-400 mg, about 400-420 mg, about 420-440 mg, about 440-460 mg, about 460-480 mg, about 480-500 mg, about 500-520 mg, about 520-540 mg, about 540-560 mg, about 560-580 mg, about 580-600 mg, about 600-620 mg, about 620-640 mg, about 640-660 mg, about 660-680 mg, about 680-700 mg, about 700-720 mg, about 720-740 mg, about 740-760 mg, about 760-780 mg, about 780-800 mg, about 800-820 mg, about 820-840 mg, about 840-860 mg, about 860-880 mg, about 880-900 mg, about 900-920 mg, about 920-940 mg, about 940-960 mg, about 960-980 mg, about 980-1000 mg, about 1000-1020 mg, about 1020-1040 mg, about 1040-1060 mg, about 1060-1080 mg, about 1080-1100 mg, about 1100-1120 mg, about 1120-1140 mg, about 1140-1160 mg, about 1160-1180 mg, about 1180-1200 mg, about 1200-1250 mg, about 1250-1300 mg, about 1300-1350 mg, about 1350-1400 mg, about 1400-1450 mg, about 1450-1500 mg, about 1500-1550 mg, about 1550-1600 mg, about 1600-1650 mg, about 1650-1700 mg, about 1700-about 1750 mg, about 1750-1800 mg, about 1800-1850 mg, about 1850-1900 mg, about 1900-1950 mg, about 1950-2000 mg, about 2000-2050 mg, about 2050-2100 mg, about 2100-2150 mg, about 2150-2200 mg, about 2200-2250 mg, about 2250-2300 mg, about 2300-2350 mg, about 2350-2400 mg, about 2400-2450 mg, about 2450-2500 mg) or more. In some embodiments, complement is inhibited for about 1 hours, 2 hours, 3, hours, 4 hours, 5 hours, 6 hours, 8 hours, 10 hours, 12, hours, 14 hours, 16 hours, 18 hours, 20 hours, 22 hours, 24 hours, 26 hours, 28 hours, 30 hours, 32 hours, 34 hours, or 36 hours after administration. In some embodiments, complement is inhibited or reduced (e.g., to a level that is 50%, 40%, 30%, 20%, 10%, 5%, or lower, relative to a control level). In some embodiments, AH50 and/or CH50 is inhibited or reduced (e.g., to a level that is 50%, 40%, 30%, 20%, 10%, 5%, or lower, relative to a control level). In some embodiments, complement is not inhibited or reduced about 6 hours, 8 hours, 10 hours, 12 hours, 14 hours, 16 hours, 18 hours, 20 hours, 22 hours, 24 hours, 26 hours, 28 hours, 30 hours, 32 hours, 34 hours, or 36 hours after administration. In some embodiments, a PEGylated compstatin analog is administered over a duration of time, e.g., as an infusion described herein, and complement is inhibited or reduced (e.g., to a level that is 50%, 40%, 30%, 20%, 10%, 5%, or lower, relative to a control level) within about 30 minutes, 45 minutes, 1 hour, or 2 hours of the initiation of administration. In some embodiments, complement is not inhibited or reduced (e.g., to a level that is 50%, 40%, 30%, 20%, 10%, 5%, or lower, relative to a control level). In some embodiments, AH50 and/or CH50 is not inhibited or reduced (e.g., to a level that is 50%, 40%, 30%, 20%, 10%, 5%, or lower, relative to a control level). In some embodiments, a control level is a level detected or measured in the same subject prior to administration of the PEGylated compstatin analog. In some embodiments, a control level is a reference level.

In some embodiments, a PEGylated compstatin analog is administered as a single dose. In some embodiments, the single dose is a bolus. In some embodiments, a bolus is an amount of a PEGylated compstatin analog that is administered, e.g., by IV infusion or other route of administration, over about 30 minutes, 20 minutes, 10 minutes, 5 minutes, or less.

In some embodiments, the single dose is an infusion. In some embodiments, an infusion is an amount of a PEGylated compstatin analog that is administered by infusion over about 15 minutes, 30 minutes, 45 minutes, 1 hour, 2 hours, 4 hours, 8 hours, 12 hours, 24 hours, 48 hours, 72 hours, or more. In some embodiments, a PEGylated compstatin analog is administered as an infusion at a rate of about 0.25 mg/min to about 45 mg/min, e.g., about 5 mg/min to about 30 mg/min, about 10 mg/min to about 20 mg/min. In some embodiments, the infusion rate is about 1 mg/min, about 3 mg/min, about 9 mg/min, about 18 mg/min, or about 20 mg/min. In some embodiments, the infusion is administered over a period of about 15 minutes to about 48 hours, e.g., about 30 minutes, about 1 hour, 2 hours, 4 hours, 8 hours, 12 hours, 18 hours, 24 hours, 36 hours, or 48 hours.

In some embodiments, a PEGylated compstatin analog is administered as two or more doses. In some embodiments, a first dose (e.g., a loading dose) and a second dose (e.g., a maintenance dose) are administered. In some embodiments, the first dose and the second dose comprise the same amount of the PEGylated compstatin analog. In some embodiments, the first dose and the second dose comprise different amounts of the PEGylated compstatin analog.

In some embodiments, the first dose comprises about 10 mg to about 600 mg of the PEGylated compstatin analog (e.g., about 10-20 mg, about 20-40 mg, about 40-60 mg, about 60-80 mg, about 80-100 mg, about 100-120 mg, about 120-140 mg, about 140-160 mg, about 160-180 mg, about 180-200 mg, about 200-220 mg, about 220-240 mg, about 240-260 mg, about 260-280 mg, about 280-300 mg, about 300-320 mg, about 320-340 mg, about 340-360 mg, about 360-380 mg, about 380-400 mg, about 400-420 mg, about 420-440 mg, about 440-460 mg, about 460-480 mg, about 480-500 mg, about 500-520 mg, about 520-540 mg, about 540-560 mg, about 560-580 mg, about 580-600 mg) and the second dose comprises about 10 mg to about 600 mg of the PEGylated compstatin analog (e.g., about 10-20 mg, about 20-40 mg, about 40-60 mg, about 60-80 mg, about 80-100 mg, about 100-120 mg, about 120-140 mg, about 140-160 mg, about 160-180 mg, about 180-200 mg, about 200-220 mg, about 220-240 mg, about 240-260 mg, about 260-280 mg, about 280-300 mg, about 300-320 mg, about 320-340 mg, about 340-360 mg, about 360-380 mg, about 380-400 mg, about 400-420 mg, about 420-440 mg, about 440-460 mg, about 460-480 mg, about 480-500 mg, about 500-520 mg, about 520-540 mg, about 540-560 mg, about 560-580 mg, about 580-600 mg).

In some embodiments, the first dose is a bolus, and the second dose is a bolus. In some embodiments, the first dose is a bolus, and the second dose is an infusion. In some embodiments, the first dose is an infusion, and the second dose is an infusion. In some embodiments, the first dose is an infusion and the second dose is a bolus. In some embodiments, one or more doses of a PEGylated compstatin analog is administered as an infusion at a rate of about 0.25 mg/min to about 45 mg/min, e.g., about 5 mg/min to about 30 mg/min, about 10 mg/min to about 20 mg/min. In some embodiments, the infusion rate is about 1 mg/min, about 3 mg/min, about 9 mg/min, about 18 mg/min, or about 20 mg/min. In some embodiments, one or more doses of a PEGylated compstatin analog is administered as an infusion over a period of about 15 minutes to about 48 hours, e.g., about 30 minutes, about 1 hour, 2 hours, 4 hours, 8 hours, 12 hours, 18 hours, 24 hours, 36 hours, or 48 hours.

In some embodiments, a subject treated with a compstatin analog described herein, e.g., a PEGylated compstatin analog described herein, has suffered a stroke, e.g., ischemic stroke. For example, it is known that reperfusion of ischemic tissue can lead to complement activation and an inflammatory response that can cause post-reperfusion injury (see, e.g., Alawieh et al., Frontiers in Immunology, Volume 6, Article 417 (2015)). Based on the results described herein, a PEGylated compstatin analog can be used to treat an ischemia-reperfusion injury, such as stroke. “Ischemia-reperfusion injury” refers to an injury that results from the re-establishment (reperfusion) of the flow of blood to a region of the body following a temporary halt in the flow. For example, ischemia-reperfusion injury can occur during certain surgical procedures, such as repair of aortic aneurysms, carotid endarterectomy, and organ transplantation. Clinically, ischemia-reperfusion injury can be manifested by complications such as, e.g., pulmonary dysfunction, including adult respiratory distress syndrome, renal dysfunction, consumptive coagulopathies including thrombocytopenia, fibrin deposition into the microvasculature and disseminated intravascular coagulopathy, transient and permanent spinal cord injury, cardiac arrhythmias and acute ischemic events, hepatic dysfunction including acute hepatocellular damage and necrosis, gastrointestinal dysfunction including hemorrhage and/or infarction and multisystem organ dysfunction (MSOD) or acute systemic inflammatory distress syndromes (SIRS). The injury may occur in the parts of the body to which the blood supply was interrupted, or it can occur in parts fully supplied with blood during the period of ischemia.

In some embodiments, methods of the disclosure include administering a PEGylated compstatin analog, e.g., according to a dosing regimen described herein, to a subject who has experienced a stroke shortly after the onset of one or more stroke symptoms, e.g., within about 5 minutes, 10 minutes, 15 minutes, 30 minutes, 1 hour, 2 hours, 3 hours, or 4 hours of onset of one or more stroke symptoms. In some embodiments, a PEGylated compstatin analog is administered as one or more doses (as described herein), e.g., for a total period of about 30 minutes, 1 hour, 2 hours, 3 hours, 4 hours, 5 hours, or 6 hours. In some embodiments, a PEGylated compstatin analog is administered in combination with (e.g., simultaneously or sequentially with) one or more additional stroke treatments. Stroke treatments that can be administered in combination with a PEGylated compstatin analog include, e.g., a thrombolytic agent (e.g., streptokinase, acylatedplasminogen-streptokinase activator complex (APSAC), urokinase, single-chain urokinase-plasminogen activator (scu-PA), anti-inflammatory agents, thrombin-like enzymes from snake venoms such as ancrod, thrombin inhibitors, tissue plasminogen activator (t-PA) and biologically active variants of each of the above); an anticoagulant (e.g., warfarin or heparin); antiplatelet drug (e.g., aspirin); a glycoprotein IIb/IIIainhibitor; a glycosaminoglycan; coumarin; GCSF; melatonin; a caspase inhibitor; an anti-oxidants (e.g., NXY-059, see Lees et al., (2006) N.Engl. J. Med 354, 588-600), a neuroprotectant (e.g., an NMDA receptor antagonist and a cannabinoid antagonist), an anti-CD18 antibody; an anti-CD11a antibody; an anti-ICAM-1 antibody; an anti-VLA-1 antibody; an anti-VLA-4 antibody, an anti-TWEAK antibody, an anti-TWEAK-R antibody, mechanical thrombectomy; carotid endarterectomy; angioplasty; and insertion of a stent. In some embodiments, a PEGylated compstatin analog is administered via a mechanical device used to treat stroke, e.g., a device used for mechanical thrombectomy. In some embodiments, one dose of the PEGylated compstatin analog is administered via a device, and one or more doses are administered using a dosing regimen described herein, e.g., a bolus and/or infusion described herein.

In some embodiments, a subject treated with a compstatin analog described herein, e.g., a PEGylated compstatin analog described herein, is undergoing dialysis. It is known that complement activation is an undesired effect of dialysis (see, e.g., Poppelaars et al., Frontiers in Immunology Volume 9, Article 71 (2018)). Based on the results described herein, a PEGylated compstatin analog can be used to inhibit complement activation, e.g., during dialysis, followed by recovery from the complement inhibition upon cessation of administration of such PEGylated compstatin analog.

In some embodiments, methods of the disclosure include administering a PEGylated compstatin analog, e.g., according to a dosing regimen described herein, to a subject who will undergo or is undergoing a dialysis procedure. In some embodiments, a PEGylated compstatin analog is administered (i) prior to the start of dialysis (e.g., about 2 hours, 1 hour, 30 minutes, or 15 minutes prior to the start of dialysis) and/or (ii) during the dialysis procedure, and/or (iii) after the end of the dialysis procedure (e.g., for about 15 minutes, 30 minutes, 1 hour, or 2 hours after the end of the dialysis procedure).

In some embodiments, a subject treated with a compstatin analog described herein, e.g., a PEGylated compstatin analog described herein, has a microangiopathy, e.g., a thrombotic microangiopathy. Thrombotic microangiopathies (TMAs) are a heterogeneous group of life-threatening disorders characterized by thrombocytopenia, schistocytosis, hemolytic anemia, microvascular thrombosis and/or end-organ damage affecting the kidney and brain. Among the major subtypes of TMAs are thrombocytopenic purpura (TTP) and hemolytic uremic syndrome (HUS). TMAs may also be pregnancy-related: either as a facet of preeclampsia, characterized by hypertension and proteinuria, or as part of the HELLP syndrome (Hemolysis, Elevated Liver enzymes and Low Platelet count).

In some embodiments, methods of the disclosure include administering a PEGylated compstatin analog, e.g., according to a dosing regimen described herein, to a subject who has or is at risk of developing a microangiopathy. In some embodiments, one or more doses of a PEGylated compstatin analog is administered at the first sign or symptom or diagnosis of microangiopathy until signs or symptoms of microangiopathy have decreased or are alleviated. In some embodiments, a PEGylated compstatin analog is administered for about 6 hours, 12 hours, 18 hours, 24 hours, 2 days, 3 days, or more. In some embodiments, a PEGylated compstatin analog is administered in combination with one or more additional treatments for microangiopathy.

In some embodiments, a subject treated with a compstatin analog described herein, e.g., a PEGylated compstatin analog described herein, has autoimmune encephalitis. Autoimmune encephalitis is diagnosed based on clinical characteristics, magnetic resonance imaging, electroencephalography, functional neuroimaging, work-up for systemic tumors, and/or detection of autoantibodies (see, e.g., Shin et al., Ther. Adv. Neurol. Disorders 11:1-19 (2018)). In some embodiments, methods of the disclosure include administering a PEGylated compstatin analog, e.g., according to a dosing regimen described herein, to a subject who has or is at risk of developing autoimmune encephalitis.

In some embodiments, a subject treated with a compstatin analog described herein, e.g., a PEGylated compstatin analog described herein, is receiving or has received a gene therapy as described herein.

In some embodiments, the age of the subject is less than 12 years. In some embodiments, the age of the subject is between 1-12 years. In some embodiments, the age of the subject is between 6-12 years. In some embodiments, the age of the subject is between 12-18 years. In some embodiments, the age of the subject is greater than 12 years. In some embodiments, the age of the subject is greater than 18 years.

IV. Gene Therapy/Viral Vectors

In some embodiments, compstatin analogs of the disclosure (e.g., PEGylated compstatin analogs) can be used to increase efficacy of a viral vector, e.g., any viral vector known in the art or described herein, and the disclosure is not limited to any particular viral vector. For example, and without wishing to be bound by theory, PEGylated compstatin analogs described herein can be used to protect viral vectors, e.g., during transit from a site of administration to a target cell or tissue; can be used to reduce formation of anti-viral vector neutralizing antibodies (e.g., that may hinder or prevent retreatment with the same viral vector); and/or can prevent or reduce inflammatory reactions that can lead, e.g., to organ damage (e.g., kidney damage). Nonlimiting examples of suitable viral vectors include, for instance, retroviral vectors (e.g., Moloney murine leukemia virus (MMLV), Harvey murine sarcoma virus, murine mammary tumor virus, Rous sarcoma virus), adenoviral vectors, adeno-associated viral (AAV) vectors, SV40-type viral vectors, polyomaviral vectors, Epstein-Barr viral vectors, papilloma viral vectors, herpes viral vectors, vaccinia viral vectors, and polio viral vectors.

Retroviruses are enveloped viruses that belong to the viral family Retroviridae. Once in a host's cell, the virus replicates by using a viral reverse transcriptase enzyme to transcribe its RNA into DNA. The retroviral DNA replicates as part of the host genome, and is referred to as a provirus. A selected nucleic acid can be inserted into a vector and packaged in retroviral particles using techniques known in the art. Protocols for the production of replication-deficient retroviruses are known in the art (see, e.g., Kriegler, M., Gene Transfer and Expression, A Laboratory Manual, W.H. Freeman Co., New York (1990) and Murry, E. J., Methods in Molecular Biology, Vol. 7, Humana Press, Inc., Cliffton, N.J. (1991)). The recombinant virus can then be isolated and delivered to cells of the subject either in vivo or ex vivo. A number of retroviral systems are known in the art, for example See U.S. Pat. Nos. 5,994,136, 6,165,782, and 6,428,953. Retroviruses include the genus of Alpha retrovirus (e.g., avian leukosis virus), the genus of Beta retrovirus; (e.g., mouse mammary tumor virus) the genus of Delta retrovirus (e.g., bovine leukemia virus and human T-lymphotropic virus), the genus of Epsilon retrovirus (e.g., Walleye dermal sarcoma virus), and the genus of Lentivirus.

In some embodiments, the retrovirus is a lentivirus of the Retroviridae family. Lentiviral vectors can transduce non-proliferating cells and show low immunogenicity. In some examples, the lentivirus is, but is not limited to, human immunodeficiency viruses (HIV-1 and HIV-2), simian immunodeficiency virus (S1V), feline immunodeficiency virus (FIV), equine infections anemia (EIA), and visna virus. Vectors derived from lentiviruses can achieve significant levels of nucleic acid transfer in vivo.

Herpes simplex virus (HSV)-based viral vectors are also suitable for use as provided herein. Many replication-deficient HSV vectors contain a deletion to remove one or more intermediate-early genes to prevent replication. Advantages of the herpes vector are its ability to enter a latent stage that can result in long-term DNA expression, and its large viral DNA genome that can accommodate exogenous DNA up to 25 kb. For a description of HSV-based vectors, see, for example, U.S. Pat. Nos. 5,837,532, 5,846,782, 5,849,572, and 5,804,413, and International Patent Applications WO 91/02788, WO 96/04394, WO 98/15637, and WO 99/06583.

In some embodiments, the vector is an adenovirus vector. Adenoviruses are a large family of viruses containing double stranded DNA. They replicate within the nucleus of a host cell, using the host's cell machinery to synthesize viral RNA, DNA and proteins. Adenoviruses are known in the art to affect both replicating and non-replicating cells, to accommodate large transgenes, and to code for proteins without integrating into the host cell genome. The virus can be made replication-deficient by deleting select genes required for viral replication. The expendable non-replication-essential E3 region is also frequently deleted to allow additional room for a larger DNA insert. The adenovirus on which a viral vector may be based may be from any origin, any subgroup, any subtype, mixture of subtypes, or any serotype. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (ATCC, Manassas, Va.). Non-group C adenoviruses, and even non-human adenoviruses, can be used to prepare replication-deficient adenoviral vectors. Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and International Patent Applications WO 97/12986 and WO 98/53087. Further examples of adenoviral vectors can be found in U.S. Publication Nos. 20150093831, 20140248305, 20120283318, 20100008889, 20090175897 and 20090088398.

In some embodiments, the viral vector is an adeno-associated virus (AAV) vector. AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012)). Methods for generating and using AAV vectors are described, for example, in U.S. Pat. Nos. 5,139,941 and 4,797,368. Several AAV serotypes have been characterized, including AAV1, AAV2, AAV3 (e.g., AAV3B), AAV4, AAV5, AAV6, AAV7, AAV8, AAV9, AAV10, and AAV11, as well as variants thereof. In some embodiments, an AAV vector is an AAV2/6, AAV2/8 or AAV2/9 vector (e.g., AAV6, AAV8 or AAV9 serotype having AAV2 ITR). Other AAV vectors are described in, e.g., Sharma et al., Brain Res Bull. Feb. 15, 2010; 81 (2-3): 273. Generally, any AAV serotype may be used to deliver a transgene described herein. However, the serotypes have different tropisms, e.g., they preferentially infect different tissues. In some embodiments, an AAV vector is a self-complementary AAV vector.

In some embodiments, an AAV vector is a naturally occurring AAV. In some embodiments, an AAV vector is a modified AAV (i.e., a variant of a naturally occurring AAV). In some embodiments, a modified AAV vector may be generated by any known vector engineering method. In some embodiments, an AAV vector may be generated by directed evolution, e.g., by DNA shuffling, peptide insertion, or random mutagenesis, in order to introduce modifications into the AAV sequence to improve one or more properties for gene therapy, e.g., to avoid or lessen an immune response or recognition by neutralizing antibodies, and/or for more efficient and/or targeted transduction (Asuri et al., Molecular Therapy 20.2 (2012): 329-338). Methods of using directed evolution to engineer an AAV vector can be found, e.g., in U.S. Pat. No. 8,632,764. In some embodiments the modified AAV is modified to include a specific tropism.

The AAV sequences of an AAV vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. In some embodiments, substantially the entire sequences encoding the ITRs are used in an AAV vector, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of an AAV vector of the present disclosure is a “cis-acting” plasmid containing a transgene, in which the selected transgene sequence and associated regulatory elements are flanked by the 5′ and 3′ AAV ITR sequences. The AAV ITR sequences may be obtained from any known AAV, including presently identified mammalian AAV types.

In some embodiments, an AAV vector may be a dual or triple AAV vector, e.g., for the delivery of large transgenes (e.g., transgenes of greater than approximately 5 kb). In some embodiments, a dual AAV vector may include two separate AAV vectors, each including a fragment of the full sequence of the large transgene of interest, and when recombined, the fragments form the full sequence of the large transgene of interest, or a functional portion thereof.

In some embodiments, a triple AAV vector may include three separate AAV vectors, each including a fragment of the sequence of the large transgene of interest, and when recombined, the fragments form the full sequence of the large transgene of interest, or a functional portion thereof.

The multiple AAV vectors of the dual or triple AAV vectors can be delivered to and co-transduced into the same cell, where the two or three fragments of transgene recombine together and generate a single mRNA transcript of the entire large transgene of interest. In some embodiments, the fragmented transgenes include a non-overlapping sequences. In some embodiments, the fragmented transgenes include a specified overlapping sequences.

In some embodiments, the multiple AAV vectors of the dual or triple may be the same type of AAV vector. In some embodiments, the multiple AAV vectors of the dual or triple may be different types of AAV vector. In some embodiments, a first AAV vector, carrying a first fragment of a large transgene of interest may include AAV1, AAV2, AAV3 (e.g., AAV3B), AAV4, AAV5, AAV6, AAV7, AAV8, AAV9, AAV10, AAV11, or a variant thereof. In some embodiments, a second AAV vector, carrying a second fragment of a large transgene of interest may include AAV1, AAV2, AAV3 (e.g., AAV3B), AAV4, AAV5, AAV6, AAV7, AAV8, AAV9, AAV10, AAV11, or a variant thereof. In some embodiments, a third AAV vector, carrying a third fragment of a large transgene of interest may include AAV1, AAV2, AAV3 (e.g., AAV3B), AAV4, AAV5, AAV6, AAV7, AAV8, AAV9, AAV10, AAV11, or a variant thereof. A viral vector may also be based on an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral transfer vectors for transgene delivery. Pseudotyped viruses may be formed by combining alphaviral envelope glycoproteins and retroviral capsids. Examples of alphaviral vectors can be found in U.S. Publication Nos. 20150050243, 20090305344, and 20060177819; the vectors and methods of their making are incorporated herein by reference in their entirety.

In addition to the major elements identified above for an AAV vector, the vector can also include conventional control elements operably linked to the transgene in a manner that permits its transcription, translation and/or expression in a cell transfected with the vector or infected with the virus produced by the disclosure. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A number of expression control sequences, including promoters that are native, constitutive, inducible and/or tissue-specific, are known in the art and may be included in a vector described herein. In some embodiments, operably linked coding sequences yield a functional RNA (e.g., miRNA).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, and the dihydrofolate reductase promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied compounds include the zinc-inducible sheep metallothionine (MT) promoter, the dexamethasone (Dex)-inducible mouse mammary tumor virus (MMTV) promoter, the T7 polymerase promoter system, the ecdysone insect promoter, the tetracycline-repressible system, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only. In another embodiment, a native promoter, or fragment thereof, for a transgene will be used. In a further embodiment, other native expression control elements, such as enhancer elements, polyadenylation sites or Kozak consensus sequences may also be used to mimic the native expression.

In some embodiments, regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. In some embodiments, the promoter is a chicken 3-actin promoter, a pol II promoter, or a pol III promoter.

In some embodiments, a viral vector is designed for expressing a transgene described herein in hepatocytes, and viral vector (e.g., an AAV vector) includes one or more liver-specific regulatory elements, which substantially limit expression of the transgene to hepatic cells. Generally, liver-specific regulatory elements can be derived from any gene known to be exclusively expressed in the liver. WO 2009/130208 identifies several genes expressed in a liver-specific fashion, including serpin peptidase inhibitor, clade A member 1, also known as α-antitrypsin (SERPINA1; GeneID 5265), apolipoprotein C—I (APOC1; GeneID 341), apolipoprotein C-IV (APOC4; GeneID 346), apolipoprotein H (APOH; GeneID 350), transthyretin (TTR; GeneID 7276), albumin (ALB; GeneID 213), aldolase B (ALDOB; GeneID 229), cytochrome P450, family 2, subfamily E, polypeptide 1 (CYP2E1; GeneID 1571), fibrinogen alpha chain (FGA; GeneID 2243), transferrin (TF; GeneID 7018), and haptoglobin related protein (HPR; GeneID 3250). In some embodiments, a viral vector described herein includes a liver-specific regulatory element derived from the genomic loci of one or more of these proteins. In some embodiments, a promoter may be the liver-specific promoter thyroxin binding globulin (TBG). Alternatively, other liver-specific promoters may be used (see, e.g., The Liver Specific Gene Promoter Database, Cold Spring Harbor, http://rulai.cshl.edu/LSPD/, such as, e.g., alpha 1 anti-trypsin (A1AT); human albumin (Miyatake et al., J. Virol. 71:5124 32 (1997)); humA1b; hepatitis B virus core promoter (Sandig et al., Gene Ther. 3:1002 9 (1996)); or LSP1. Additional vectors and regulatory elements are described in, e.g., Baruteau et al., J. Inherit. Metab. Dis. 40:497-517 (2017)).

Methods for obtaining viral vectors are known in the art. For example, to produce AAV vectors, the methods typically involve culturing a host cell which contains a nucleic acid sequence encoding an AAV capsid protein or fragment thereof, a functional rep gene; a recombinant AAV vector composed of AAV inverted terminal repeats (ITRs) and a transgene; and/or sufficient helper functions to permit packaging of the recombinant AAV vector into the AAV capsid proteins.

The components to be cultured in a host cell to package an AAV vector in an AAV capsid may be provided to the host cell in trans. Alternatively, any one or more of the required components (e.g., recombinant AAV vector, rep sequences, cap sequences, and/or helper functions) may be provided by a stable host cell that has been engineered to contain one or more of the required components using methods known to those of skill in the art. In some embodiments, such a stable host cell contains the required component(s) under the control of an inducible promoter. In other embodiments, the required component(s) may be under the control of a constitutive promoter. In other embodiments, a selected stable host cell may contain selected component(s) under the control of a constitutive promoter and other selected component(s) under the control of one or more inducible promoters. For example, a stable host cell may be generated that is derived from 293 cells (which contain E1 helper functions under the control of a constitutive promoter), but that contain the rep and/or cap proteins under the control of inducible promoters. Other stable host cells may be generated by one of skill in the art using routine methods.

Recombinant AAV vector, rep sequences, cap sequences, and helper functions required for producing an AAV of the disclosure may be delivered to a packaging host cell using any appropriate genetic element (e.g., vector). A selected genetic element may be delivered by any suitable method known in the art, e.g., to those with skill in nucleic acid manipulation and include genetic engineering, recombinant engineering, and synthetic techniques (see, e.g., Sambrook et al, Molecular Cloning: A Laboratory Manual, Cold Spring Harbor Press, Cold Spring Harbor, N.Y.). Similarly, methods of generating AAV virions are well known and any suitable method can be used with the present disclosure (see, e.g., K. Fisher et al, J. Virol., 70:520-532 (1993) and U.S. Pat. No. 5,478,745).

In some embodiments, recombinant AAVs may be produced using a triple transfection method (e.g., as described in U.S. Pat. No. 6,001,650). In some embodiments, recombinant AAVs are produced by transfecting a host cell with a recombinant AAV vector (comprising a transgene) to be packaged into AAV particles, an AAV helper function vector, and an accessory function vector. An AAV helper function vector encodes “AAV helper function” sequences (i.e., rep and cap), which function in trans for productive AAV replication and encapsidation. In some embodiments, the AAV helper function vector supports efficient AAV vector production without generating any detectable wild-type AAV virions (i.e., AAV virions containing functional rep and cap genes). Non-limiting examples of vectors suitable for use with the present disclosure include pHLP19 (see, e.g., U.S. Pat. No. 6,001,650) and pRep6cap6 vector (see, e.g., U.S. Pat. No. 6,156,303). An accessory function vector encodes nucleotide sequences for non-AAV derived viral and/or cellular functions upon which AAV is dependent for replication (i.e., “accessory functions”). Accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any known helper viruses such as adenovirus, herpesvirus (other than herpes simplex virus type-1), and vaccinia virus.

In some embodiments, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art (see, e.g., Graham et al. (1973) Virology, 52:456; Sambrook et al. (1989) Molecular Cloning, a laboratory manual, Cold Spring Harbor Laboratories, New York, Davis et al. (1986) Basic Methods in Molecular Biology, Elsevier; and Chu et al. (1981) Gene 13:197). Such techniques can be used to introduce one or more exogenous nucleic acids, such as a nucleotide integration vector and other nucleic acid molecules, into suitable host cells.

In some embodiments, a host cell is a mammalian cell. A host cell may be used as a recipient of an AAV helper construct, an AAV minigene plasmid, an accessory function vector, and/or other transfer DNA associated with the production of recombinant AAVs. The term includes the progeny of an original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

Additional methods for generating and isolating AAV viral vectors suitable for delivery to a subject are described in, e.g., U.S. Pat. Nos. 7,790,449; 7,282,199; WO 2003/042397; WO 2005/033321, WO 2006/110689; and U.S. Pat. No. 7,588,772. In one system, a producer cell line is transiently transfected with a construct that encodes the transgene flanked by ITRs and a construct(s) that encodes rep and cap. In another system, a packaging cell line that stably supplies rep and cap is transiently transfected with a construct encoding the transgene flanked by ITRs. In each of these systems, AAV virions are produced in response to infection with helper adenovirus or herpesvirus, and AAVs are separated from contaminating virus. Other systems do not require infection with helper virus to recover the AAV—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In yet another system, the transgene flanked by ITRs and rep/cap genes are introduced into insect host cells by infection with baculovirus-based vectors. Such production systems are known in the art (see generally, e.g., Zhang et al., 2009, Human Gene Therapy 20:922-929). Methods of making and using these and other AAV production systems are also described in U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The foregoing methods for producing recombinant vectors are not meant to be limiting, and other suitable methods will be apparent to the skilled artisan.

V. Transgenes

The transgene of a viral vector described herein may be a gene therapy transgene and may encode any protein or portion thereof beneficial to a subject, such as one with a disease or disorder. The protein may be an extracellular, intracellular or membrane-bound protein. The protein can be a therapeutic protein. In some embodiments, the subject to whom the gene therapy is administered has a disease or disorder whereby the subject's endogenous version of the protein is defective or produced in limited amounts or not at all. In some such embodiments, the transgene encodes a non-defective version of the protein. In some embodiments, the subject to whom the gene therapy is administered has a disease or disorder mediated by a target gene (e.g., by a level of expression of the target gene and/or level of activity of a target polypeptide), and the transgene encodes an inhibitor of the target gene or target polypeptide. Examples of therapeutic proteins include, but are not limited to, infusible or injectable therapeutic proteins, enzymes, enzyme cofactors, hormones, blood or blood coagulation factors, cytokines and interferons, growth factors, adipokines, etc.

Examples of infusible or injectable therapeutic proteins include, for example, Tocilizumab (Roche/Actemra®), VEGF inhibitors, alpha-1 antitrypsin (Kamada/AAT), Hematide® (Affymax and Takeda, synthetic peptide), albinterferon alfa-2b (Novartis/Zalbin™) Rhucin® (Pharming Group, C1 inhibitor replacement therapy), tesamorelin (Theratechnologies/Egrifta, synthetic growth hormone-releasing factor), ocrelizumab (Genentech, Roche and Biogen), belimumab (GlaxoSmithKline/Benlysta®), pegloticase (Savient Pharmaceuticals/Krystexxa™), taliglucerase alfa (Protalix/Uplyso), agalsidase alfa (Shire/Replagal®), and velaglucerase alfa (Shire).

Examples of enzymes include lysozyme, oxidoreductases, transferases, hydrolases, lyases, isomerases, asparaginases, uricases, glycosidases, proteases, nucleases, collagenases, hyaluronidases, heparinases, heparanases, kinases, phosphatases, lysins and ligases. Other examples of enzymes include those that used for enzyme replacement therapy including, but not limited to, imiglucerase (e.g., CEREZYME™), α-galactosidase A (a-gal A) (e.g., agalsidase beta, FABRYZYME™), acid a-glucosidase (GAA) (e.g., alglucosidase alfa, LUMIZYME™ MYOZYME™), and arylsulfatase B (e.g., laronidase, ALDURAZYME™, idursulfase, ELAPRASE™, arylsulfatase B, NAGLAZYME™)

Examples of hormones include, but are not limited to, gonadotropins, thyroid-stimulating hormone, melanocortins, pituitary hormones, vasopressin, oxytocin, growth hormones, prolactin, orexins, natriuretic hormones, parathyroid hormone, calcitonins, erythropoietin, and pancreatic hormones.

Examples of blood or blood coagulation factors include Factor I (fibrinogen), Factor II (prothrombin), tissue factor, Factor V (proaccelerin, labile factor), Factor VII (stable factor, proconvertin), Factor VIII (antihemophilic globulin), Factor IX (Christmas factor or plasma thromboplastin component), Factor X (Stuart-Prower factor), Factor Xa, Factor XI, Factor XII (Hageman factor), Factor XIII (fibrin-stabilizing factor), von Willebrand factor, von Heldebrant Factor, prekallikrein (Fletcher factor), high-molecular weight kininogen (HMWK) (Fitzgerald factor), fibronectin, fibrin, thrombin, antithrombin, such as antithrombin III, heparin cofactor II, protein C, protein S, protein Z, protein Z-related protease inhibitor (ZPI), plasminogen, alpha 2-antiplasmin, tissue plasminogen activator (tPA), urokinase, plasminogen activator inhibitor-1 (PAI1), plasminogen activator inhibitor-2 (PAI2), cancer procoagulant, and epoetin alfa (Epogen, Procrit).

Examples of cytokines include lymphokines, interleukins, and chemokines, type 1 cytokines, such as IFN-γ, TGF-β, and type 2 cytokines, such as IL-4, IL-10, and IL-13.

Examples of growth factors include Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha (TGFα), Transforming growth factor beta (TGFβ), Tumour necrosis factor-alpha (TNFα), Vascular endothelial growth factor (VEGF), Wnt Signaling Pathway, placental growth factor (PlGF), [(Foetal Bovine Somatotrophin)] (FBS), IL-1, IL-2, IL-3, IL-4, IL-5, IL-6, and IL-7. Examples of adipokines include leptin and adiponectin.

Additional examples of therapeutic proteins include, but are not limited to, receptors, signaling proteins, cytoskeletal proteins, scaffold proteins, transcription factors, structural proteins, membrane proteins, cytosolic proteins, binding proteins, nuclear proteins, secreted proteins, golgi proteins, endoplasmic reticulum proteins, mitochondrial proteins, and vesicular proteins, etc.

The transgene of a gene therapy viral vector provided herein may encode a functional version of any protein that, through some defect in the endogenous version in a subject (including a defect in the expression of an endogenous version), results in a disease or disorder in the subject. In some embodiments, the endogenous version of a protein in a subject may be absent or underexpressed. Examples of such diseases or disorders include, but are not limited to, lysosomal storage diseases/disorders, such as Santavuori-Haltia disease (Infantile Neuronal Ceroid Lipofuscinosis Type 1), Jansky-Bielschowsky Disease (late infantile neuronal ceroid lipofuscinosis, Type 2), Batten disease (juvenile neuronal ceroid lipofuscinosis, Type 3), Kufs disease (neuronal ceroid lipofuscinosis, Type 4), Von Gierke disease (glycogen storage disease, Type Ia), glycogen storage disease, Type Ib, Pompe disease (glycogen storage disease, Type II), Forbes or Cori disease (glycogen storage disease, Type III), mucolipidosis II (I-Cell disease), mucolipidosis III (Pseudo-Hurler polydystrophy), mucolipidosis IV (sialolipidosis), cystinosis (adult nonnephropathic type), cystinosis (infantile nephropathic type), cystinosis (juvenile or adolescent nephropathic), Salla disease/infantile sialic acid storage disorder, and saposin deficiencies; disorders of lipid and sphingolipid degradation, such as GM1 gangliosidosis (infantile, late infantile/juvenile, and adult/chronic), Tay-Sachs disease, Sandhoff disease, GM2 gangliosidosis, Ab variant, Fabry disease, Gaucher disease, Types I, II and III, metachromatic leukodystrophy, Krabbe disease (early and late onset), Neimann-Pick disease, Types A, B, C1, and C2, Farber disease, and Wolman disease (cholesteryl esther storage disease); disorders of mucopolysaccharide degradation, such as Hurler syndrome (MPSI), Scheie syndrome (MPS IS), Hurler-Scheie syndrome (MPS IH/S), Hunter syndrome (MPS II), Sanfillippo A syndrome (MPS IIIA), Sanfillippo B syndrome (MPS IIIB), Sanfillippo C syndrome (MPS IIIC), Sanfillippo D syndrome (MPS IIID), Morquio A syndrome (MPS IVA), Morquio B syndrome (MPS IVB), Maroteaux-Lamy syndrome (MPS VI), and Sly syndrome (MPS VII); disorders of glycoprotein degradation, such as alpha mannosidosis, beta mannosidosis, fucosidosis, asparylglucosaminuria, mucolipidosis I (sialidosis), galactosialidosis, Schindler disease, and Schindler disease, Type II/Kanzaki disease; and leukodystrophy diseases/disorders, such as abetalipoproteinemia, neonatal adrenoleukodystrophy, Canavan disease, cerebrotendinous xanthromatosis, Pelizaeus Merzbacher disease, Tangier disease, Refum disease, infantile, and Refum disease, classic.

Additional examples of such diseases/disorders of a subject as provided herein include, but are not limited to, acid maltase deficiency (e.g., Pompe disease, glycogenosis type 2, lysosomal storage disease); carnitine deficiency; carnitine palmityl transferase deficiency; debrancher enzyme deficiency (e.g., Cori or Forbes disease, glycogenosis type 3); lactate dehydrogenase deficiency (e.g., glycogenosis type 11); myoadenylate deaminase deficiency; phosphofructokinase deficiency (e.g., Tarui disease, glycogenosis type 7); phosphogylcerate kinase deficiency (e.g., glycogenosis type 9); phosphogylcerate mutase deficiency (e.g., glycogenosis type 10); phosphorylase deficiency (e.g., McArdle disease, myophosphorylase deficiency, glycogenosis type 5); Gaucher's Disease (e.g., chromosome 1, enzyme glucocerebrosidase affected); Achondroplasia (e.g., chromosome 4, fibroblast growth factor receptor 3 affected); Huntington's Disease (e.g., chromosome 4, huntingtin); Hemochromatosis (e.g., chromosome 6, HFE protein); Cystic Fibrosis (e.g., chromosome 7, CFTR); Friedreich's Ataxia (chromosome 9, frataxin); Best Disease (chromosome 11, VMD2); Sickle Cell Disease (chromosome 11, hemoglobin); Phenylketonuria (chromosome 12, phenylalanine hydroxylase); Marfan's Syndrome (chromosome 15, fibrillin); Myotonic Dystrophy (chromosome 19, dystrophia myotonica protein kinase); Adrenoleukodystrophy (x-chromosome, lignoceryl-CoA ligase in peroxisomes); Duchene's Muscular Dystrophy (x-chromosome, dystrophin); Rett Syndrome (x-chromosome, methylCpG-binding protein 2); Leber's Hereditary Optic Neuropathy (mitochondria, respiratory proteins); Mitochondria Encephalopathy, Lactic Acidosis and Stroke (MELAS) (mitochondria, transfer RNA); and Enzyme deficiencies of the Urea Cycle.

Still additional examples of such diseases or disorders include, but are not limited to, Sickle Cell Anemia, Myotubular Myopathy, Hemophilia B, Lipoprotein lipase deficiency, Ornithine Transcarbamylase Deficiency, Crigler-Najjar Syndrome, Mucolipidosis IV, Niemann-Pick A, Sanfilippo A, Sanfilippo B, Sanfilippo C, Sanfilippo D, b-thalassemia and Duchenne Muscular Dystrophy. Still further examples of diseases or disorders include those that are the result of defects in lipid and sphingolipid degradation, mucopolysaccharide degradation, glycoprotein degradation, leukodystrophies, etc.

The functional versions of the defective proteins of any one of the disease or disorders provided herein may be encoded by the transgene of a gene therapy viral vector and are also considered therapeutic proteins. Therapeutic proteins also include Myophosphorylase, glucocerebrosidase, fibroblast growth factor receptor 3, huntingtin, HFE protein, CFTR, frataxin, VMD2, hemoglobin, phenylalanine hydroxylase, fibrillin, dystrophia myotonica protein kinase, lignoceryl-CoA ligase, dystrophin, methylCpG-binding protein 2, Beta hemoglobin, Myotubularin, Cathepsin A, Factor IX, Lipoprotein lipase, Beta galactosidase, Ornithine Transcarbamylase, Iduronate-2-Sulfatase, Acid-Alpha Glucosidase, UDP-glucuronosyltransferase 1-1, GlcNAc-1-phosphotransferase, GlcNAc-1-phosphotransferase, Mucolipin-1, Microsomal triglyceride transfer protein, Sphingomyelinase, Acid ceramidase, Lysosomal acid lipase, Alpha-L-iduronidase, Heparan N-sulfatase, alpha-N-acetylglucosaminidase, acetyl-CoA alpha-glucosaminide acetyltransferase, N-acetylglucosamine 6-sulfatase, N-acetylgalactosamine-6 sulfatase, Alpha-mannosidase, Alpha-galactosidase A, Cystic fibrosis conductance transmembrane regulator, and respiratory proteins.

As further examples, therapeutic proteins also include functional versions of proteins associated with disorders of lipid and sphingolipid degradation (e.g., β-Galactosidase-1, β-Hexosaminidase A, β-Hexosaminidases A and B, GM2 Activator Protein, 8-Galactosidase A, Glucocerebrosidase, Glucocerebrosidase, Glucocerebrosidase, Arylsulfatase A, Galactosylceramidase, Sphingomyelinase, Sphingomyelinase, NPC1, HE1 protein (Cholesterol Trafficking Defect), Acid Ceramidase, Lysosomal Acid Lipase); disorders of mucopolysaccharide degradation (e.g., L-Iduronidase, L-Iduronidase, L-Iduronidase, Iduronate Sulfatase, Heparan N-Sulfatase, N-Acetylglucosaminidase, Acetyl-CoA-Glucosaminidase, Acetyltransferase, Acetylglucosamine-6-Sulfatase, Galactosamine-6-Sulfatase, Arylsulfatase B, Glucuronidase); disorders of glycoprotein degradation (e.g., Mannosidase, mannosidase, 1-fucosidase, Aspartylglycosaminidase, Neuraminidase, Lysosomal protective protein, Lysosomal 8-N-acetylgalactosaminidase, Lysosomal 8-N-acetylgalactosaminidase); lysosomal storage disorders (e.g., Palmitoyl-protein thioesterase, at least 4 subtypes, Lysosomal membrane protein, Unknown, Glucose-6-phosphatase, Glucose-6-phosphate translocase, Acid maltase, Debrancher enzyme amylo-1,6 glucosidase, N-acetylglucosamine-1-phosphotransferase, N-acetylglucosamine-1-phosphotransferase, Ganglioside sialidase (neuraminidase), Lysosomal cystine transport protein, Lysosomal cystine transport protein, Lysosomal cystine transport protein, Sialic acid transport protein Saposins, A, B, C, D) and leukodystrophies (e.g., Microsomal triglyceride transfer protein/apolipoprotein B, Peroxisomal membrane transfer protein, Peroxins, Aspartoacylase, Sterol-27-hydroxlase, Proteolipid protein, ABC1 transporter, Peroxisome membrane protein 3 or Peroxisome biogenesis factor 1, Phytanic acid oxidase).

The viral vectors provided herein may be used for gene editing. In such embodiments, the transgene of the viral vector is a gene editing transgene. Such a transgene encodes an agent or component that is involved in a gene editing process. Generally, such a process results in long-lasting or permanent modifications to genomic DNA, such as targeted DNA insertion, replacement, mutagenesis or removal. Gene editing may include the delivery of nucleic acids encoding a DNA sequence of interest and inserting the sequence of interest at a targeted site in genomic DNA using endonucleases. Thus, gene editing transgenes may comprise these nucleic acids encoding a DNA sequence of interest for insertion. In some embodiments, the DNA sequence for insertion is a DNA sequence encoding any one of the therapeutic proteins described herein. Additionally or alternatively, the gene editing transgene may comprise nucleic acids that encode one of more components that can alone or in combination with other components carry out the gene editing process. The gene editing transgenes provided herein may encode an endonuclease and/or a guide RNA, etc.

Endonucleases can create breaks in double-stranded DNA at desired locations in a genome and use the host cell's mechanisms to repair the break using homologous recombination, nonhomologous end-joining, etc. Classes of endonucleases that can be used for gene editing include, but are not limited to, meganucleases (see, e.g., U.S. Pat. Nos. 8,802,437, 8,445,251 and 8,338,157; and U.S. Publication Nos. 20130224863, 20110113509 and 20110033935), zinc-finger nucleases (ZFNs) (see, e.g., U.S. Pat. Nos. 8,956,828; 8,921,112; 8,846,578; 8,569,253), transcription activator-like effector nucleases (TALENs) (see, e.g., U.S. Pat. No. 8,697,853; as well as U.S. Publication Nos. 20150118216, 20150079064, and 20140087426), clustered regularly interspaced short palindromic repeat(s) (CRISPR) and homing endonucleases (see, e.g., U.S. Publication No. 20150166969; and U.S. Pat. No. 9,005,973).

The gene editing transgene of the viral vectors provided herein may encode any one of the endonucleases provided herein. For example, the CRISPR (clustered regularly interspaced short palindromic repeats)/Cas system can be used for gene editing. In a CRISPR/Cas system, guide RNA (gRNA) is encoded genomically or episomally (e.g., on a plasmid). The gRNA forms a complex with an endonuclease, such as Cas9 endonuclease, following transcription. The complex is then guided by the specificity determining sequence (SDS) of the gRNA to a DNA target sequence, typically located in the genome of a cell. Cas9 or Cas9 endonuclease refers to an RNA-guided endonuclease comprising a Cas9 protein, or a fragment thereof (e.g., a protein comprising an active or inactive DNA cleavage domain of Cas9 or a partially inactive DNA cleavage domain (e.g., a Cas9 nickase), and/or the gRNA binding domain of Cas9). Cas9 recognizes a short motif in the CRISPR repeat sequences (the PAM or protospacer adjacent motif) to help distinguish self versus non-self Cas9 endonuclease and guide RNA (e.g., single guide RNA) sequences and structures are well known to those of skill in the art (see, e.g., Ferretti et al., Proc. Natl. Acad. Sci. U.S.A. 98:4658-4663(2001); Deltcheva et al., Nature 471:602-607(2011); and Jinek et al., Science 337:816-821(2012)).

The viral vectors provided herein may be used for gene expression modulation. In such embodiments, the transgene of the viral vector is a gene expression modulating transgene. Such a transgene encodes a gene expression modulator that can enhance, inhibit (e.g., silence) or modulate the expression of one or more endogenous genes. The endogenous gene may encode any one of the proteins as provided herein provided the protein is an endogenous protein of the subject. Accordingly, the subject may be one with any one of the diseases or disorders provided herein where there would be a benefit provided by gene expression modulation.

Gene expression modulators include DNA-binding proteins (e.g., artificial transcription factors, such as those of U.S. Publication No. 20140296129; and transcriptional silencer protein NRF of U.S. Publication No. 20030125286) as well as therapeutic RNAs. Therapeutic RNAs include, but are not limited to, inhibitors of mRNA translation (antisense) (see, e.g., U.S. Publication Nos. 20050020529 and 20050271733), agents of RNA interference (RNAi) (see, e.g., U.S. Pat. Nos. 8,993,530, 8,877,917, 8,293,719, 7,947,659, 7,919,473, 7,790,878, 7,737,265, 7,592,322; and U.S. Publication Nos. 20150197746, 20140350071, 20140315835, 20130156845 and 20100267805), catalytically active RNA molecules (ribozymes) (see, e.g., Hasselhoff, et al., Nature, 334:585, 1988; and U.S. Publication No. 20050020529), transfer RNA (tRNA) and RNAs that bind proteins and other molecular ligands (aptamers). Gene expression modulators include any agents of the foregoing and include antisense nucleic acids, RNAi molecules (e.g., double-stranded RNAs (dsRNAs), single-stranded RNAs (ssRNAs), micro RNAs (miRNAs), short interfering RNAs (siRNAs), short hairpin RNAs (shRNAs)) and triplex-forming oligonucleotides (TFOs). Gene expression modulators also may include modified versions of any of the foregoing RNA molecules and, thus, include modified mRNAs, such as synthetic chemically modified RNAs.

Exemplary transgenes encode interfering RNA, antisense RNA, ribozymes, and aptamers that decrease the level of an angiogenic factor in a cell. For example, an RNAi can be a miRNA, an shRNA, or an siRNA that reduces the level of vascular endothelial growth factor (VEGF) in a cell. For example, an RNAi can be an shRNA or siRNA that reduces the level of VEGF or VEGF receptor (VEGFR) in a cell. RNAi agents that target VEGF include, e.g., an RNAi described in U.S. Patent Publication No. 2011/0224282. For example, an siRNA specific for VEGF-A, VEGFR1, or VEGFR2 would be suitable. Suitable nucleic acid gene products also include a ribozyme specific for VEGF-A, VEGFR1, or VEGFR2; an antisense specific for VEGF-A, VEGFR1, or VEGFR2; siRNA specific for VEGF-A, VEGFR1, or VEGFR2; etc. Also suitable as a gene product is an miRNA that reduces the level of VEGF by regulating VEGF gene expression, e.g., through post-transcriptional repression or mRNA degradation. Examples of suitable miRNA include, e.g., miR-15b, miR-16, miR-20a, and miR-20b. See, e.g., Hua et al. (2006) PLoS ONE 1:e116. Also suitable is an anti-VEGF aptamer (e.g., EYE001). For anti-VEGF aptamers, see, e.g., Ng et al. (2006) Nature Reviews Drug Discovery 5:123; and U.S. Pat. Nos. 6,426,335; 6,168,778; 6,147,204; 6,051,698; and 6,011,020. For example, an aptamer can be directed against VEGF165, the isoform primarily responsible for pathological ocular neovascularization and vascular permeability.

In some embodiments, a transgene encodes a polypeptide (e.g., an antibody or fusion protein) that inhibits or reduces activity of one or more polypeptides described herein. For example, in some embodiments, a transgene encodes an anti-angiogenic polypeptide including, e.g., vascular endothelial growth factor (VEGF) antagonists. Suitable VEGF antagonists include, but are not limited to, inhibitors of VEGFR1 tyrosine kinase activity; inhibitors of VEGFR2 tyrosine kinase activity; an antibody to VEGF; an antibody to VEGFR1; an antibody to VEGFR2; a soluble VEGFR; and the like (see, e.g., Takayama et al. (2000) Cancer Res. 60:2169-2177; Mori et al. (2000) Gene Ther. 7:1027-1033; and Mahasreshti et al. (2001) Clin. CancerRes. 7:2057-2066; and U.S. Patent Publication No. 20030181377). Antibodies specific for VEGF include, e.g., bevacizumab (AVASTIN™) and ranibizumab (also known as rhuFAb V2). Also suitable for use are anti-angiogenic polypeptides such as endostatin, PEDF, and angiostatin.

Anti-angiogenic polypeptides include, e.g., recombinant polypeptides comprising VEGF receptors. For example, a suitable anti-angiogenic polypeptide can be the soluble form of the VEGFR-1, known as sFlt-1 (Kendall et al. (1996) Biochem. Biophys. Res. Commun.226:324). Suitable anti-angiogenic polypeptides also include an immunoglobulin-like (Ig) domain 2 of a first VEGF receptor (e.g., Flt1), alone or in combination with an Ig domain 3 of a second VEGF receptor (e.g., Flk1 or Flt4); an anti-angiogenic polypeptide can also include a stabilization and/or a multimerization component. Such recombinant anti-angiogenic polypeptides are described in, e.g., U.S. Pat. No. 7,521,049. Anti-VEGF antibodies that are suitable as heterologous gene products include single chain Fv (scFv) antibodies. See, e.g., U.S. Pat. Nos. 7,758,859; and 7,740,844, for anti-VEGF antibodies. Additional transgenes are described in, e.g., Bordet et al., Drug Discov Today. Jun. 5, 2019. pii: S1359-6446(18)30472-0. doi: 10.1016/j.drudis.2019.05.038. Such transgenes can be used to treat ocular disorders, e.g., age-related macular degeneration.

In some embodiments, the gene therapy comprises an adeno-associated virus, serotype 9 (AAV9) vector comprising a truncated human dystrophin gene (mini-dystrophin) under the control of a muscle specific promoter. In some embodiments, the mini-dystrophin gene comprises a 6-8 kb sequence of the full length dystrophin gene. In some embodiments, the mini-dystrophin gene may contain a fragment of the full dystrophin gene sequence, while still containing the minimal amount of information required for production of function dystrophin protein. In some embodiments, the mini-dystrophin genes contain at least the R16/R17 nNOS binding domain sequence of the dystrophin gene (e.g., as described in Zhang et al. Human Gene Ther. 2012 January; 23(1): 98-103). For example, in some embodiments the gene therapy may be PF-06939926. The gene therapy may be administered, e.g., by IV infusion, to a subject suffering from Duchenne muscular dystrophy (DMD). In some embodiments, the mini-dystrophin gene therapy may be delivered using more than one AAV vector, such as a dual or triple AAV vector.

In some embodiments, the gene therapy comprises an AAV9 vector comprising a micro-dystrophin gene under the control of a muscle specific promoter. The micro-dystrophin gene may contain a fragment of the full dystrophin gene sequence, while still containing the minimal amount of information required for production of function dystrophin protein. In some embodiments, a micro-dystrophin gene may comprise the sequence of the dystrophin gene with one or more of the R1-R24 segments of spectrin-like repeats removed. For example, in some embodiments the gene therapy may be SGT-001. In some embodiments, the micro-dystrophin gene may include ΔDysM3 is the first synthetic micro-dystrophin. Δ3990, ΔR4-23/ΔC and μDys5R (as described in Duan 2018 Molecular Therapy 26(10)). In some embodiments, the micro-dystrophin gene therapy may be delivered using more than one AAV vector, each containing a different micro-dystrophin gene. The gene therapy may be administered, e.g., by IV infusion, to a subject suffering from DMD.

In some embodiments, the gene therapy comprises an adeno-associated virus, serotype 5 (AAV5) vector comprising a human Factor IX gene. The gene therapy may be administered, e.g., by IV infusion, to a subject suffering from hemophilia B. In some embodiments, a gene therapy vector encodes a functional version that has gain-of-function activity (e.g., increased expression or activity relative to wild type). For example, for hemophilia involving lack of Factor IX, a Factor IX variant that contains the gain-of-function mutation known as Padua (R338L) which leads to an enhancement of expression of Factor IX, may be used. For example, in some embodiments, the gene therapy is AMT-061.

In some embodiments, the gene therapy comprises an AAV Serotype 8 (AAV8) vector comprising a transgene encoding human ornithine transcarbamylase (OTC). The gene therapy may be administered, e.g., by IV infusion, to subjects suffering from OTC deficiency. In some embodiments the gene therapy is DTX301.

In some embodiments, the gene therapy comprises an adeno-associated virus, serotype 5 (AAV5) vector comprising a human Factor VIII gene. The gene therapy may be administered, e.g., by IV infusion, to a subject suffering from hemophilia A. In some embodiments, the gene therapy is Valoctocogene Roxaparvovec.

In some embodiments, the gene therapy comprises an AAV8 vector comprising the human GAA gene under the control of the LSP promoter. The gene therapy may be administered, e.g., by IV infusion, to a subject suffering from Pompe disease.

In some embodiments, the gene therapy comprises an AAV8 vector comprising a transgene encoding human glucose-6-phosphatase (G6Pase). The gene therapy may be administered, e.g., by IV infusion, to a subject suffering from glycogen storage disease type Ia (GSDIa).

In some embodiments, the gene therapy comprises an adeno-associated virus serotype 9 (AAV9) vector comprising a transgene encoding the human lysosome-associated membrane protein 2 isoform B (LAMP2B) (e.g., RP-A501). In some embodiments, the gene therapy is administered, e.g., by IV infusion, to a subject suffering from Danon Disease.

In some embodiments, the gene therapy comprises an AAV vector, e.g., an AAV8 vector, comprising a transgene that encodes human LDL receptor. The gene therapy may be administered, e.g., by IV infusion, to a subject suffering from familial hypercholesterolemia (e.g., homozygous familial hypercholesterolemia). In some embodiments, the gene therapy comprises AAV8.TBG.hLDLR.

In some embodiments, the gene therapy comprises an adeno-associated virus serotype 2 vector (AAV2) comprising a normal human CHM gene (encoding REP1). The gene therapy may be administered sub-retinally to a subject suffering from or at risk of choroideremia.

In some embodiments, the gene therapy comprises an AAV2 vector comprising a transgene that encodes human RPE65, e.g., AAV2-hRPE65v2. The gene therapy may be administered, e.g., subretinally, to a subject suffering from Leber Congenital Amaurosis.

In some embodiments, the gene therapy comprises an adeno-associated virus 2/6 (AAV2/6) vector encoding a B-domain deleted human Factor VIII (e.g., SB-525). In some embodiments, the gene therapy comprises an adeno-associated virus 8 vector encoding a B-domain deleted human Factor VIII (e.g., BAX 888). The gene therapy providing B-domain deleted human Factor VIII may be administered, e.g., by IV infusion, to a subject suffering from hemophilia A.

In some embodiments, the gene therapy comprises an AAV9 vector comprising a human CLN3 transgene. The therapy may be administered, e.g., intrathecally, to a subject having a CLN3 mutation. In some embodiments, the gene therapy comprises an AAV9 vector comprising a human CLN6 transgene. The therapy may be administered, e.g., intrathecally, to a subject having a CLN6 mutation.

In some embodiments, the gene therapy comprises an AAV8 vector containing a functional copy of the human MTM1 (hMTM1) gene (e.g., AT132). The gene therapy may be administered, e.g., by IV infusion, to a subject suffering from X-Linked Myotubular Myopathy.

In some embodiments, a viral vector described herein includes (i) a transgene described herein and (ii) a nucleic acid encoding a complement inhibitor described herein.

The sequence of a transgene may also include an expression control sequence. Expression control DNA sequences include promoters, enhancers, and operators, and are generally selected based on the expression systems in which the expression construct is to be utilized. In some embodiments, promoter and enhancer sequences are selected for the ability to increase gene expression, while operator sequences may be selected for the ability to regulate gene expression. The transgene may also include sequences that facilitate, and preferably promote, homologous recombination in a host cell. The transgene may also include sequences that are necessary for replication in a host cell.

Exemplary expression control sequences include promoter sequences, e.g., cytomegalovirus promoter; Rous sarcoma virus promoter; and simian virus 40 promoter; as well as any other types of promoters that are disclosed elsewhere herein or are otherwise known in the art. Generally, promoters are operatively linked upstream (i.e., 5′) of the sequence coding for a desired expression product. The transgene also may include a suitable polyadenylation sequence (e.g., the SV40 or human growth hormone gene polyadenylation sequence) operably linked downstream (i.e., 3′) of the coding sequence.

VI. Pharmaceutical Compositions

Complement inhibitors, e.g., PEGylated compstatin analogs, described herein can be incorporated into pharmaceutical compositions. Such pharmaceutical compositions are useful for, among other things, administration and delivery to a subject in vivo or ex vivo. In some embodiments, pharmaceutical compositions also contain a pharmaceutically acceptable carrier or excipient. Such excipients include any pharmaceutical agent, e.g., a pharmaceutical agent that does not itself induce an immune response harmful to the individual receiving the composition, and which may be administered without undue toxicity. As used herein the terms “pharmaceutically acceptable” and “physiologically acceptable” mean a biologically acceptable formulation, gaseous, liquid or solid, or mixture thereof, which is suitable for one or more routes of administration, in vivo delivery or contact. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

Pharmaceutical compositions may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In some embodiments, a pharmaceutical composition may be a lyophilized powder.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration can comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, mannitol, or dextran. Additionally, suspensions of the active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility to allow for the preparation of highly concentrated solutions. In some embodiments, pharmaceutical compositions include sodium acetate (e.g., about 5 mM to about 30 mM, e.g., about 10 mM), NaCl (e.g., about 0.5% to about 2%, e.g., about 0.9%) and water, and have a pH of about 4 to about 7, e.g., about 5. In some embodiments, prior to IV administration, such pharmaceutical compositions are diluted in isotonic saline.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Pharmaceutical compositions and delivery systems appropriate for the compositions, methods and uses of the disclosure are known in the art (see, e.g., Remington: The Science and Practice of Pharmacy. 21st Edition. Philadelphia, Pa. Lippincott Williams & Wilkins, 2005).

PEGylated compstatin analogs described herein can be administered by any suitable route. The route and/or mode of administration can vary depending upon the desired results. Methods and uses of the disclosure include delivery and administration systemically, regionally or locally, or by any route, for example, by injection or infusion. The mode of administration is left to the discretion of the practitioner. Delivery of a pharmaceutical composition in vivo may generally be accomplished via injection using a conventional syringe, although other delivery methods such as convection-enhanced delivery can also be used (see, e.g., U.S. Pat. No. 5,720,720). For example, compositions may be delivered subcutaneously, epidermally, epidurally, intracerebrally, intradermally, intranasally, intrathecally, intraorbitally, intramucosally, intraperitoneally, intravenously, intra-pleurally, subretinally, intraarterially, sublingually, intrahepatically, via the portal vein, and intramuscularly. In some embodiments, administration is via intravenous infusion, e.g., central or peripheral intravenous infusion. Other modes of administration include oral and pulmonary administration, suppositories, and transdermal applications. A clinician may determine the optimal route for administration.

VII. Combination Therapy

In some aspects, methods of the present disclosure involve administering a PEGylated compstatin analog to a subject receiving, or who has previously received, gene therapy (e.g., a viral vector described herein). In some methods, a PEGylated compstatin analog and a gene therapy (e.g., a viral vector) are administered to a subject.

In some embodiments, the PEGylated compstatin analog is administered to a subject who has received or is concurrently or sequentially receiving one or more doses of gene therapy. In some embodiments, the gene therapy is a viral vector, e.g., an AAV vector. In some embodiments, a subject has received gene therapy 1 day, 1 week, 2 weeks, 4 weeks, 2 months, 4 months, 6 months, or more prior to administration of a PEGylated compstatin analog.

In some embodiments, a PEGylated compstatin analog is administered to a subject who has not been receiving gene therapy. In some embodiments, a subject has not received gene therapy for about 1 day, 1 week, 2 weeks, 4 weeks, 2 months, 4 months, 6 months, 1 year, 2 years, 5 years, or more prior to administration of a PEGylated compstatin analog. In some embodiments, the subject has never received gene therapy.

In some embodiments, a PEGylated compstatin analog and a gene therapy are administered to a subject. In some embodiments, the PEGylated compstatin analog and the gene therapy are administered concurrently (e.g., within about 1 minute, 5 minutes, 10 minutes, 15 minutes, 30 minutes, 45 minutes, 1 hour, or 2 hours of each other). In some embodiments, the PEGylated compstatin analog and the gene therapy are administered sequentially (e.g., more than 1 hour, 6 hours, 12 hours, 18 hours, 24 hours, 2 days, 3 days, 4 days, 5 days, 6 days, 7 days, 2 weeks, 4 weeks, or more, apart).

In some embodiments, a subject is pretreated with a PEGylated compstatin analog before receiving a gene therapy. In some embodiments, a PEGylated compstatin analog is administered to a subject 2, 4, 6, 8, 10, 12, 14, 16, 18, 20, or 24 hours before a gene therapy dose. In some embodiments, a PEGylated compstatin analog is administered to a subject more that 24 hours before a gene therapy dose.

In some embodiments, a subject may be administered a short course treatment of PEGylated compstatin analog. In some embodiments, a short course treatment may include a single dose of PEGylated compstatin analog. In some embodiments, a short course treatment may include up to 2, 3, 4, 5, 6, 7, 8, 9, or 10 doses.

In some embodiments, a short course treatment is a treatment regimen in which the number of days between the first and last dose administered (including the day the first and last dose were administered) is 1, 2, 3, 4, 5, 6, or 7 days. In some embodiments, a short course treatment is a treatment regimen in which the number of days between the first and last dose administered (including the day the first and a last dose were administered) is up to 10, 14, 21, or 28 days.

In some embodiments, the PEGylated compstatin analog is initially administered followed by a period in which the PEGylated compstatin analog is not administered to the subject for a longer time period, e.g., 6 months, 1 year, 2, 3, 4, 5 years, or more. In some embodiments, a subject who receives a gene therapy may be administered a single course of PEGylated compstatin analog therapy. In some embodiments a subject may be administered a second (or subsequent) course of PEGylated compstatin analog therapy in conjunction with receiving a second (or subsequent) dose (or doses) of a gene therapy.

In some embodiments, a short course treatment of a PEGylated compstatin analog may inhibit complement for a relatively short period of time (e.g., up to 12 or 24 hours, or up to 1, 2, 3, 4, 5, 6, or 7 days or up to 1, 2, 3, 4, 5, or 6 weeks) during a time in which one or more dose(s) of a gene therapy, e.g., an AAV vector, is administered to a subject.

In some embodiments, a PEGylated compstatin analog is administered for a period of time in order to inhibit complement until the viral vector of the gene therapy is taken up by one or more target cells (e.g., taken up by a target cell by a certain level or extent). In some embodiments, cell uptake is indicated or measured in a subject using an assay to detect a decreased level of viral vector in a sample obtained from the subject (e.g., a serum sample) and/or increased level of viral vector in one or more target cells. Level of viral vector can be measured by any known method for detecting a viral vector in a sample (e.g., ELISA, PCR, etc.).

In some embodiments, a short course treatment may include an IV administration (e.g., by IV infusion) that lasts for, e.g., between 15 minutes and 48 hours, e.g., 30 minutes, 1 hour, 2 hour, 4-8 hours, 8-16 hours, 16-24 hours, 24-48 hours, or 48-72 hours. In some embodiments, the short course treatment by IV infusion may span the time in which a subject receives gene therapy. In some embodiments, the short course treatment by IV infusion may be before a subject receives gene therapy. In some embodiments, the short course treatment by IV infusion may be after a subject receives gene therapy.

In some embodiments, a subject may be administered a short course treatment of PEGylated compstatin analog before receiving a gene therapy. In some embodiments, a short course of PEGylated compstatin analog treatment may be administered between 15 minutes and 48 hours, e.g., 30 minutes, 1 hour, 2 hour, 4-8 hours, 8-16 hours, 16-24 hours, 24-48 hours, or 48-72 hours, before a subject receives a gene therapy treatment.

In some embodiments, a subject may be administered a short course treatment of PEGylated compstatin analog after receiving a gene therapy. In some embodiments, a short course of PEGylated compstatin analog treatment may be administered between 15 minutes and 48 hours, e.g., 30 minutes, 1 hour, 2 hour, 4-8 hours, 8-16 hours, 16-24 hours, 24-48 hours, or 48-72 hours after a subject has received a gene therapy treatment.

In some embodiments, a PEGylated compstatin analog is administered in conjunction with immunosuppressive therapy.

In some embodiments, the combination therapy results in improved of the gene therapy (e.g., an improvement in a disease or disorder described herein or a symptom thereof) in a subject over a specified time period (e.g., over 1 week, 2 weeks, 3 weeks, 4 weeks, 6 weeks, 8 weeks, 3 months, 4 months, 6 months, 12 months, 24 months, 3 years, 4 years, 5 years, or more), relative to a subject receiving only the gene therapy. In some embodiments, gene therapy is administered to a subject receiving a PEGylated compstatin analog. In some embodiments, a PEGylated compstatin analog is administered to a subject receiving gene therapy.

In some embodiments, the combination therapy results in reduced side effects of the gene therapy (e.g., reduced immune response to a viral vector) in a subject over a specified time period (e.g., over 1 week, 2 weeks, 3 weeks, 4 weeks, 6 weeks, 8 weeks, 3 months, 4 months, 6 months, 12 months, 24 months, 3 years, 4 years, 5 years, or more), relative to a subject receiving only the gene therapy. In some embodiments, gene therapy is administered to a subject receiving a PEGylated compstatin analog. In some embodiments, a PEGylated compstatin analog is administered to a subject receiving gene therapy.

In some embodiments, administration of a PEGylated compstatin analog allows a subject who has received a first dose of a gene therapy (e.g., a viral vector) to receive one or more additional doses of gene therapy. In some embodiments, the one or more additional doses of gene therapy may be administered at least 6 months, e.g., at least 1, 2, 3, 5, or more years after the first dose of the gene therapy.

In some embodiments, a PEGylated compstatin analog is administered with a first dose of viral vector, e.g., AAV vector. In some such embodiments, the initial combination allows administration of one or more additional doses of the AAV vector.

In some embodiments, the one or more additional dose(s) of gene therapy comprise(s) a gene therapy using a viral vector of the same serotype as the gene therapy of the first dose. In some embodiments the one or more additional dose(s) of gene therapy comprise(s) a gene therapy using a viral vector with the same viral capsid and/or envelope as the viral vector in the first dose.

In some embodiments, the one or more additional dose(s) of gene therapy comprise(s) a gene therapy using a viral vector of a different serotype than the viral vector used in the first dose. In some embodiments the one or more additional dose(s) of gene therapy comprise(s) a gene therapy using a viral vector with a different viral capsid and/or envelope than the viral vector used in the first dose.

In some embodiments, the one or more additional dose(s) of gene therapy comprise a gene therapy using the same transgene as the first dose. In some embodiments, the one or more additional dose(s) of gene therapy comprises a gene therapy using a different transgene than the first dose. In some embodiments, the subject does not have detectable preexisting antibodies to the gene therapy (e.g., viral vector).

In some embodiments, the age of the subject is less than 12 years. In some embodiments, the age of the subject is between 1-12 years. In some embodiments, the age of the subject is between 6-12 years. In some embodiments, the age of the subject is between 12-18 years. In some embodiments the age of the subject is greater than 12 years. In some embodiments, the age of the subject is greater than 18 years.

In some embodiments, combined administration of a PEGylated compstatin analog described herein and a gene therapy described herein results in an improvement in a disease or disorder described herein or a symptom thereof to an extent that is greater than one produced by either the gene therapy or the PEGylated compstatin analog alone. The difference between the combined effect and the effect of the gene therapy or PEGylated compstatin analog alone can be a statistically significant difference. In some embodiments, the combined result is synergistic.

In some embodiments, combined administration of a PEGylated compstatin analog and gene therapy allows administration of the gene therapy at a reduced dose, at a reduced number of doses, and/or at a reduced frequency of dosage compared to an effective dosing regimen for the gene therapy alone, and/or compared to a standard dosing regimen approved for the gene therapy. In some embodiments, combined administration of a PEGylated compstatin analog and the gene therapy allows administration of the PEGylated compstatin analog at a reduced dose, at a reduced number of doses, and/or at a reduced frequency of dosage compared to an effective dosing regimen for the PEGylated compstatin analog alone.

In some embodiments, efficacy of gene therapy and/or PEGylated compstatin analog is assessed at, e.g., 1 week, 2 weeks, 3 weeks, 4 weeks, 6 weeks, 8 weeks, 3 months, 6 months, or longer after administration of therapy.

In some embodiments, efficacy of gene therapy and/or PEGylated compstatin analog is measured or indicated by a disease sign or symptom recurrence-free period relative to a subject receiving only gene therapy or PEGylated compstatin analog. In some embodiments, efficacy of gene therapy and/or PEGylated compstatin analog is measured or indicated by increased time to recurrence of a disease sign or symptom relative to a subject receiving only gene therapy or PEGylated compstatin analog.

In some embodiments, efficacy of gene therapy and/or PEGylated compstatin analog is measured or indicated by a decrease in humoral response and/or a decrease in cellular response relative to a subject receiving only gene therapy and/or PEGylated compstatin analog. In some embodiments, decrease in humoral response is measured or indicated by decrease in magnitude of response or fold decrease from baseline of antibody (e.g., neutralizing antibody) levels. In some embodiments, antibody level is level of antibody against viral vector, e.g., capsid protein. In some embodiments, baseline is a value, level, amount or quantity measured or indicated in a subject (having a disease or disorder described herein) prior to administration of gene therapy and/or PEGylated compstatin analog. In some embodiments, baseline is a value, level, amount or quantity measured or indicated in a subject (not having a disease or disorder described herein) prior to administration of gene therapy and/or PEGylated compstatin analog. In some embodiments, decreased humoral response is indicated by a decrease in antibody titer from baseline of at least 10%, 20%, 30%, 40%, 50%, 60%, 70%, 80%, 90%, or 100%.

In some embodiments, cellular response is indicated or measured by secretion of granzyme B (GrB) and/or IFNγ. In some embodiments, decrease in cellular response is measured or indicated by decrease in magnitude of response or fold decrease from baseline of GrB and/or IFNγ levels. In some embodiments, decreased cellular response is indicated by a decrease in GrB and/or IFNγ levels from baseline of at least 10%, 20%, 30%, 40%, 50%, 60%, 70%, 80%, 90%, or 100%. In some embodiments, baseline is a value, level, amount or quantity measured or indicated in a subject (having a disease or disorder described herein) prior to administration of gene therapy and/or PEGylated compstatin analog. In some embodiments, baseline is a value, level, amount or quantity measured or indicated in a subject (not having a disease or disorder described herein) prior to administration of gene therapy and/or PEGylated compstatin analog.

In some embodiments, efficacy of gene therapy is measured by level of presence or expression of a transgene described herein, and/or level or activity of a protein encoded by a transgene described herein. For example, combined therapy of a PEGylated compstatin analog and gene therapy results in a level of transgene in a subject (e.g., in a cell or tissue of the subject) at, e.g., 1 week, 2 weeks, 3 weeks, 4 weeks, 6 weeks, 8 weeks, 3 months, 6 months, or longer after combined therapy, that is at least 10%, 20%, 30%, 40%, 50%, 60%, 70%, 80%, 90%, 100%, 125%, 150%, 175%, 200%, or more, higher relative to a corresponding level of transgene in a subject not administered the PEGylated compstatin analog. In some embodiments, combined therapy of a PEGylated compstatin analog and gene therapy results in a level of expression of transgene (and/or level of activity of a protein encoded by the transgene) in a subject (e.g., in a cell or tissue of the subject) at, e.g., 1 week, 2 weeks, 3 weeks, 4 weeks, 6 weeks, 8 weeks, 3 months, 6 months, or longer after combined therapy, that is at least 10%, 20%, 30%, 40%, 50%, 60%, 70%, 80%, 90% 100%, 125%, 150%, 175%, 200%, or more, higher relative to a corresponding level of expression and/or activity in a subject not administered the PEGylated compstatin analog.

In some embodiments, the efficacy of gene therapy is measured by a stable level of expression of the transgene in the subject over a period of, e.g., 1 week, 2 weeks, 3 weeks, 4 weeks, 6 weeks, 8 weeks, 3 months, 6 months, or longer, relative to a corresponding level of expression of the transgene over the same period in a control subject (e.g., a control subject receiving the gene therapy and not administered the PEGylated compstatin analog). In some embodiments, a stable level of expression is a level of expression that differs by no more than 30%, 25%, 20%, 15%, 10%, or 5% over a defined period of time.

In some embodiments, efficacy of gene therapy with a transgene that encodes an inhibitor of a target gene or polypeptide is measured by level of expression of a target gene and/or level of expression and/or activity of a target polypeptide. In some embodiments, combined therapy of a PEGylated compstatin analog and gene therapy results in a level of expression of a target gene and/or level of expression and/or activity of a target polypeptide in a subject (e.g., in a cell or tissue of the subject) at, e.g., 1 week, 2 weeks, 3 weeks, 4 weeks, 6 weeks, 8 weeks, 3 months, 6 months, or longer after combined therapy, that is at least 10%, 20%, 30%, 40%, 50%, 60%, 70%, 80%, 90%, or 100% lower relative to a corresponding level of expression and/or activity in a subject not administered the PEGylated compstatin analog.

Gene therapy and/or PEGylated compstatin analog can be administered by any suitable route. The route and/or mode of administration can vary depending upon the desired results. Methods and uses of the disclosure include delivery and administration systemically, regionally or locally, or by any route, for example, by injection or infusion. The mode of administration is left to the discretion of the practitioner. Delivery of a pharmaceutical composition in vivo may generally be accomplished via injection using a conventional syringe, although other delivery methods such as convection-enhanced delivery can also be used (see, e.g., U.S. Pat. No. 5,720,720). For example, compositions may be delivered subcutaneously, epidermally, epidurally, intracerebrally, intradermally, intranasally, intrathecally, intraorbitally, intramucosally, intraperitoneally, intravenously, intra-pleurally, subretinally, intraarterially, sublingually, intrahepatically, via the portal vein, and intramuscularly. Other modes of administration include oral and pulmonary administration, suppositories, and transdermal applications. A clinician may determine the optimal route for administration.

In some embodiments, gene therapy and PEGylated compstatin analog are administered by the same route. In some embodiments, gene therapy and PEGylated compstatin analog are administered by different routes.

The disclosure also provides methods for introducing viral vectors and PEGylated compstatin analog described herein into a cell or an animal. In some embodiments, such methods include contacting a subject (e.g., a cell or tissue of a subject) with, or administering to a subject (e.g., a subject such as a mammal), a PEGylated compstatin analog and a viral vector (e.g., an AAV vector) comprising a transgene such that the transgene is expressed in the subject (e.g., in a cell or tissue of a subject). In another embodiment, a method includes providing cells of an individual (patient or subject such as a mammal) with a PEGylated compstatin analog and a viral vector (e.g., an AAV vector) comprising a transgene described herein, such that the transgene is expressed in the individual.

Compositions of a vector (e.g., an AAV vector) comprising a transgene described herein can be administered in a sufficient or effective amount to a subject in need thereof. Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

The dose to achieve a therapeutic effect, e.g., the dose in vector genomes/per kilogram of body weight (vg/kg), will vary based on several factors including, but not limited to: route of administration, the level of transgene expression required to achieve a therapeutic effect, the specific disease treated, and the stability of the transgene expressed. One skilled in the art can determine an AAV/vector genome dose range to treat a patient having a particular disease or disorder based on the aforementioned factors, as well as other factors. Generally, doses will range from at least 1×10⁸, or more, for example, 1×10⁹, 1×10¹⁰, 1×10¹¹, 1×10¹², 1×10¹³, 1×10¹⁴, or more, vector genomes per kilogram (vg/kg) of the weight of the subject, to achieve a therapeutic effect.

An effective amount or a sufficient amount can (but need not) be provided in a single administration, or may require multiple administrations. For example, the amount may be proportionally increased as indicated by the need of the subject, type, status and severity of the disease treated or side effects (if any) of treatment. Determining a therapeutically effective dose is well within the capability of a skilled medical practitioner using the techniques and guidance provided in the disclosure.

In some embodiments, administration of a PEGylated compstatin analog described herein can reduce the amount and/or activity of C3 in the subject's blood sufficiently such that efficacy of a gene therapy is enhanced, as described herein.

In some embodiments the dose of PEGylated compstatin analog is administered as a single daily dose, e.g., subcutaneously. In some embodiments a dose of PEGylated compstatin analog is administered as a single weekly dose, e.g., subcutaneously. In some embodiments, a PEGylated compstatin analog is administered to a subject 2, 4, 6, 8, 10, 12, 14, 16, 18, 20, or 24 hours before a gene therapy dose.

In some embodiments, a subject is monitored before and/or following treatment with a PEGylated compstatin analog for level of C3 expression and/or activity, e.g., as measured using an alternative pathway assay, a classical pathway assay, or both. Suitable assays are known in the art and include, e.g., a hemolysis assay. In some embodiments, a subject is treated with a PEGylated compstatin analog, or is retreated with a PEGylated compstatin analog, if a measured level of C3 expression and/or activity is more than 10%, 20%, 30%, 40%, 50%, 100%, 200%, or more, relative to measured level of C3 expression and/or activity in a control subject.

In some embodiments, a dosing regimen may be selected to achieve a selected level of complement inhibition (e.g., inhibition of the alternative pathway, classical pathway, or both), for a selected time period. In some embodiments the selected level of complement inhibition is at least 50%, 60%, 70%, 80%, 90%, or more. In some embodiments the selected time period may be, e.g., between 8 hours and 72 hours, e.g., between 12 hours and 48 hours, e.g., about 16, 20, 24, 28, 32, 36, 40, or 44 hours. In some embodiments the selected time period may be, e.g., between 44 and 72 hours, or between 72 and 128 hours.

In some embodiments, a method may comprise obtaining one or more samples, e.g., blood samples, from a subject to whom a PEGylated compstatin analog has been administered and measuring the level of complement inhibition (e.g., expression and/or activity of C3) using one or more assays. In some embodiments, PEGylated compstatin analog therapy may be continued until a selected level of inhibition has been achieved. Following achievement of such selected level of inhibition, one or more doses of a gene therapy may be administered. The subject may continue to receive PEGylated compstatin analog therapy during and/or following administration of the one or more doses(s) of gene therapy.

In some embodiments, a method may comprise alternatively or additionally monitoring a subject for signs and/or symptoms associated with complement activation during or following administration of a gene therapy. In some embodiments, e.g., if one or more signs or symptoms of complement activation is detected, e.g., if complement activation at or above a reference level is detected, the dose of PEGylated compstatin analog may be increased or one or more additional doses may be administered. In some embodiments, e.g., if one or more signs of complement activation is not detected, e.g., if complement activation remains below a reference level, the dose of PEGylated compstatin analog may be decreased or maintained at the same level or no additional doses may be administered. In some embodiments the reference level may be, e.g., a level of complement activation measured in that particular subject prior to administration of the gene therapy, typically when the subject is not suffering from an infection or other stimulus that may activate complement. In some embodiments the reference level may be an upper limit of the normal range as measured in the general population.

In some embodiments, the dose of PEGylated compstatin analog administered during a period of administration may remain the same or approximately the same during the period of administration. In some embodiments the dose administered during such period of administration may change during the period of administration.

In some embodiments, a subject may be administered a loading dose of a PEGylated compstatin analog, which loading dose is followed by administration of one or more maintenance doses, e.g., at a different dosage level. In some embodiments a loading dose contains a greater amount of PEGylated compstatin analog than one or more maintenance doses.

In some embodiments, the loading dose may be determined based on the level of complement activity (e.g., C3). In some embodiments, one or more maintenance dose may be determined based on the level of complement activity (e.g., C3).

In some embodiments, the loading dose is administered before administration of a gene therapy to a subject. In some embodiments, a loading dose of PEGylated compstatin analog is administered to a subject 2, 4, 6, 8, 10, 12, 14, 16, 18, 20, or 24 hours before a gene therapy dose. In some embodiments, a loading dose of PEGylated compstatin analog is administered to a subject 2, 4, 6, 8, 10, 12, 14, 16, 18, 20, or 24 hours before a gene therapy dose. In some embodiments, the loading dose is administered after administration of a gene therapy to a subject. In some embodiments, at least one maintenance dose is administered before administration of a gene therapy to a subject. In some embodiments, at least one maintenance dose is administered after administration of a gene therapy to a subject. In some embodiments, a subsequent dose of gene therapy (e.g., AAV vector) is administered after at least one maintenance dose. In some embodiments, at least one subsequent maintenance dose is administered following a subsequent dose of gene therapy.

In some embodiments, a maintenance dose may include one or more dose(s) of PEGylated compstatin analog. In some embodiments, a maintenance dose consists of multiple doses administered over a period time of between 8 hours and 72 hours, e.g., between 12 hours and 48 hours, e.g., about 16, 20, 24, 28, 32, 36, 40, 44, or 48 hours. In some embodiments the selected time period may be, e.g., between 44 and 72 hours, or between 72 and 128 hours.

In some embodiments, a maintenance dose may include an IV administration (e.g., by IV infusion) that lasts for, e.g., between 15 minutes and 48 hours, e.g., 30 minutes, 1 hour, 2 hour, 4-8 hours, 8-16 hours, 16-24 hours, 24-48 hours, or 48-72 hours. In some embodiments, the maintenance dose by IV infusion may span the time in which a subject receives gene therapy.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

### Example 1—Dose Escalation Study of Exemplary PEGylated Compstatin Analog

### Methods

Healthy human volunteers were divided into 6 cohorts, and were administered the PEGylated compstatin analog depicted in FIG. 1, having a PEG of about 10 kD, by IV infusion according to the following dosing schedules:

| | | Approximate Infusion |
|---|---|---|
| Cohort | Dosing Schedule | Rate & Duration |
| 1 | Single IV infusion of 30 mg | 1 mg/min for 30 min |
| | or placebo | |
| 2 | Single IV infusion of 90 mg | 3 mg/min for 30 min |
| | or placebo | |
| 3 | Single IV infusion of 270 mg | 9 mg/min for 30 min |
| | or placebo | |
| 4 | Single IV infusion of 540 mg | 18 mg/min for 30 min |
| | or placebo | |
| 5 | Single IV infusion of 540 mg | 0.75 mg/min (45 mg/hour) |
| | or placebo | for 12 hours |
| 6 | Single IV infusion of 600 mg | 16 mg/min (960 mg/hour) |
| | or matching placebo | for 15 min, then 0.25 |
| | | mg/min (15 mg/hour) for |
| | | 24 hours |

Time Points for Complement Analysis

Blood samples for complement analysis were collected during screening (between Day −28 and Day −3), at predose (Day 1), and at 1 hour (Day 1), 2 Hours (Day 1), 4 Hours (Day 1), 8 Hours (Day 1), 12 Hours (Day 1), 24 Hours (Day 2), 48 Hours (Day 3), 72 Hours (Day 4), 96 Hours (Day 5), 120 Hours (Day 6), 144 Hours (Day 7), 168 Hours (Day 8), 240 Hours (Day 11), 336 Hours (Day 15), 408 Hours (Day 18), 504 Hours (Day 22), 576 Hours (Day 25), 672 Hours (Day 29), and 1008 Hours (Day 43) following dosing.

C3 Level

C3 levels were measured on the BindingSite SPA PLUS instrument. The SPA PLUS measures protein concentration by a turbidimetric immunoassay. The standards, controls and specimens to be tested were mixed with a fixed concentration of excess polyclonal antibodies to the analyte of interest. This led to the formation of large light-scattering antigen-antibody immune complexes. The amount of light transmitted through this solution was then measured and compared to a standard curve to determine the concentration of analyte in the specimen.

For turbidimetric assays, the amount of analyte in a sample is inversely proportional to the amount of light transmitted and is dependent on the amount of agglutination between the analyte and the specific antibodies. Standards containing a known amount of analyte were measured and the SPA PLUS generated a standard curve with optical density (OD) as the independent variable (Y) and concentration as the dependent variable (X). From this standard curve, regression analysis was performed to calculate the amount of analyte in unknown samples (controls and specimens).

C3a Split Product Testing

C3a was measured by ELISA (MicroVue C3a Plus EIA, lot number 129694; Quidel Corporation, San Diego Ca) using microtiter plates precoated with specific monoclonal antibodies against C3a. The standards, controls and test specimens were diluted and placed in duplicate into the wells and incubated to allow binding of the C3a split product to the antibodies in the well. After washing away unbound proteins, a second anti-C3a antibody, conjugated to horseradish peroxidase (HRP) was allowed to react with the C3a bound to the first antibody on the plate. After an appropriate incubation time and washing, a chromogenic substrate for HRP was added to the wells and the optical density (OD) of the reaction product was determined spectrophotometrically at wavelength 450 nm. Three QC specimens were run on every assay (high, medium and low C3a level).

The optical densities (OD) for each of the wells containing the standards for the assay were entered as the dependent variables (Y) in a linear regression calculation (WorkOut 2.5) in which the concentrations of the standards (provided by the kit manufacturer, Quidel) were the independent variables (X). The data was fit with a 4-parameter parametric curve and the concentrations for the unknowns (controls and test specimens) were obtained from that standard curve.

CH50 Method

CH50 was measured by a hemolytic assay based on lysis of antibody-coated sheep red blood cells (EA) due to activation of complement classical pathway on the cell's surface. Serial dilutions (1/50, 1/75, 1/112.5, 1/168.8 and 1/253) of the test specimen were mixed with equal volumes of the EA. This functional assay measured, at wavelength 415 nm, the amount of hemoglobin that was released when the target cells were lysed by the action of complement, and from these values, the percentage of the cells that had been lysed was calculated. For each assay the run was verified with a five-point standard and five dilutions of characterized quality control material.

For all samples, the percent lysis at each dilution in the five-point dilution series was entered as the dependent variable (Y) in a linear regression calculation (Microsoft Excel) and the reciprocal of the dilutions used were entered as the independent variables (X). The slope (m) and intercept (b) obtained from the regression analysis were then used to determine the 50% lysis point (the point where the best-fit line formed from the percent lysis versus the reciprocal of the dilution of serum reaches 50%). The CH50 value is the reciprocal of the dilution of serum required to lyse 50% of the cells in the assay and was expressed in Units/mL. CH50 activity as % of baseline was determined by dividing the measurement (time points) after treatment by the measurement before treatment (baseline is defined as Day 1: pre-dose) multiplied by 100. The screening time point was also expressed as % baseline.

AH50 Method

AH50 was measured by a hemolytic assay based on the lysis of rabbit red blood cells due to activation of complement alternative pathway on the cell's surface. For this assay, the buffers that were used block the complement classical pathway and only allow for activity of the alternative pathway. Serial dilutions (1/5, 1/7, 1/9.8, 1/13.7 and 1/19.2) of the serum were mixed with equal volumes of the rabbit red blood cells. This functional assay measured, at 415 nm, the amount of hemoglobin that was released when the target cells were lysed by the action of complement, and from these values the percentage of cells that had been lysed was calculated. For each assay the run was verified with a five-point standard and five dilutions of characterized quality control material.

For all samples, the percent lysis at each dilution in the five-point dilution series was entered as the dependent variable (Y) in a linear regression calculation (Microsoft Excel) and the reciprocal of the dilutions used were entered as the independent variables (X). The slope (m) and intercept (b) obtained from the regression analysis were then used to determine the 50% lysis point (the point where the best-fit line formed from the percent lysis versus the reciprocal of the dilution of serum reaches 50%). The AH50 value is the reciprocal of the dilution of serum required to lyse 50% of the cells in the assay and was expressed in Units/mL. AH50 activity as % of baseline was determined by dividing the measurement (time points) after treatment by the measurement before treatment (baseline is defined as Day 1: pre-dose) multiplied by 100. The screening time point was also expressed as % baseline.

Results

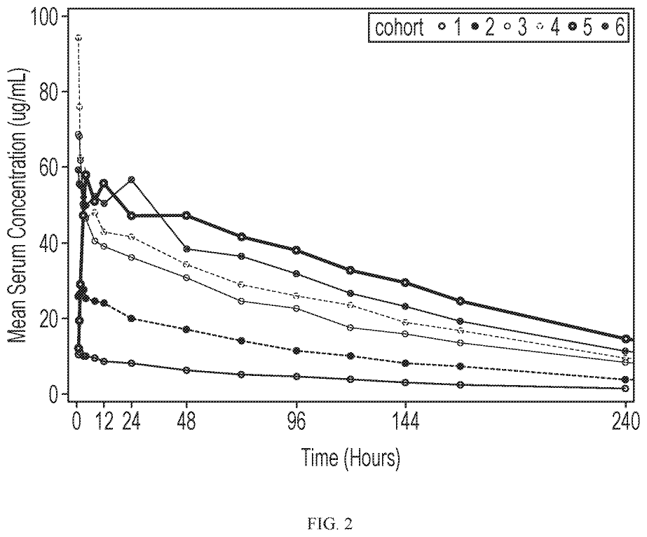

FIG. 2 depicts the mean serum PEGylated compstatin analog concentration (μg/mL) for the cohorts. Calculated Cmax and tmax are shown in the following table:

Cmax:

| Cohort | N | Mean | Std Dev | Median | Min | Max | CV (%) |
|---|---|---|---|---|---|---|---|
| Cohort 1 | 4 | 11.27 | 3.25 | 10.38 | 8.5 | 15.8 | 28.86% |
| Cohort 2 | 4 | 28.28 | 4.25 | 26.50 | 25.5 | 34.6 | 15.02% |
| Cohort 3 | 4 | 68.60 | 10.64 | 72.60 | 53.0 | 76.2 | 15.51% |
| Cohort 4 | 4 | 94.38 | 13.74 | 93.45 | 78.6 | 112.0 | 14.55% |
| Cohort 5 | 4 | 69.80 | 4.45 | 68.70 | 66.0 | 75.8 | 6.37% |
| Cohort 6 | 4 | 60.18 | 7.05 | 57.5 | 55.1 | 70.6 | 11.71% |

t_max:

| Cohort | N | Mean | Std Dev | Median | Min | Max | CV (%) |
|---|---|---|---|---|---|---|---|
| Cohort 1 | 4 | 1.63 | 0.48 | 1.75 | 1.0 | 2.0 | 29.46% |
| Cohort 2 | 4 | 2.13 | 1.03 | 2.25 | 1.0 | 3.0 | 48.51% |
| Cohort 3 | 4 | 1.00 | 0 | 1.00 | 1.0 | 1.0 | 0% |
| Cohort 4 | 4 | 0.99 | 0.0085 | 1.00 | 0.983 | 1.00 | 0.85% |
| Cohort 5 | 4 | 21.00 | 18.00 | 12.01 | 12.0 | 48.0 | 85.68% |
| Cohort 6 | 4 | 1.38 | 0.47 | 1.27 | 1.0 | 2.0 | 34.00% |

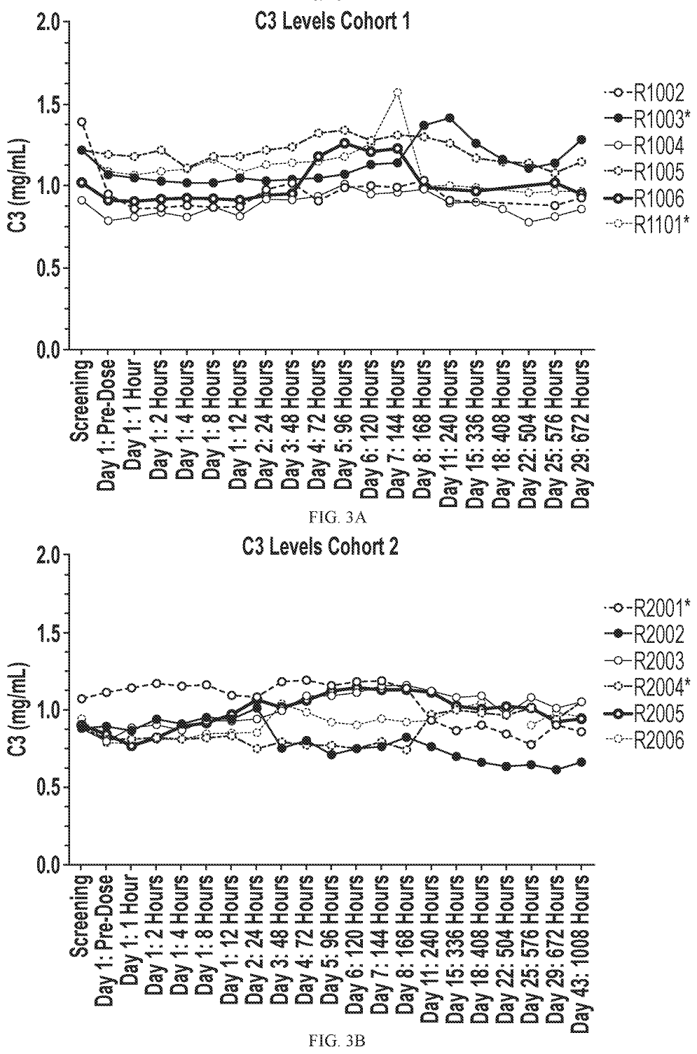

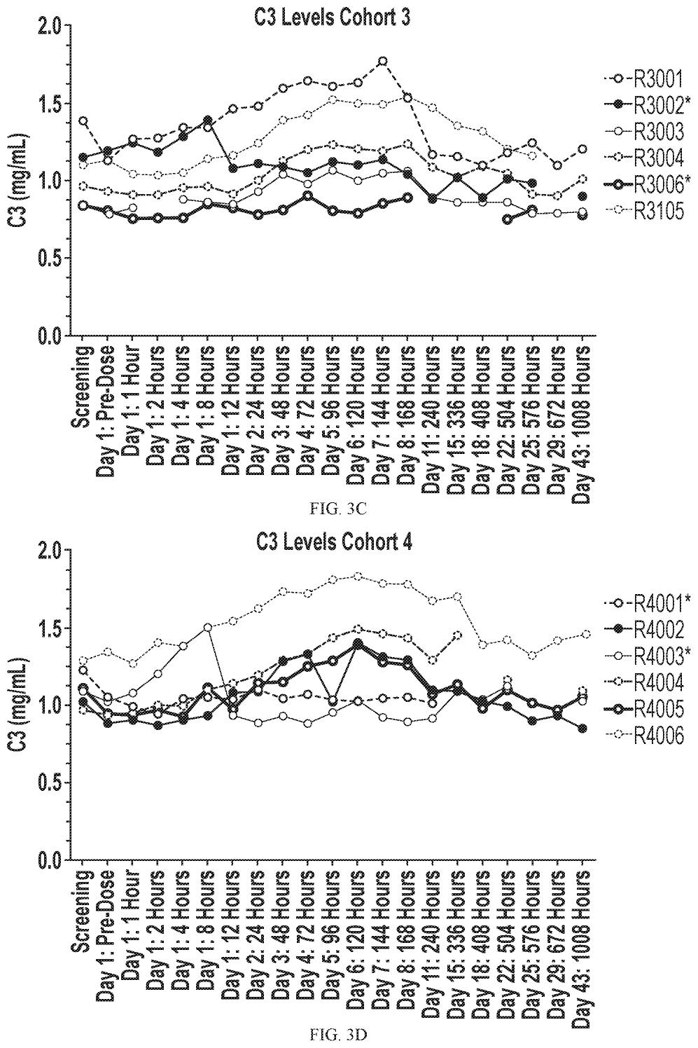

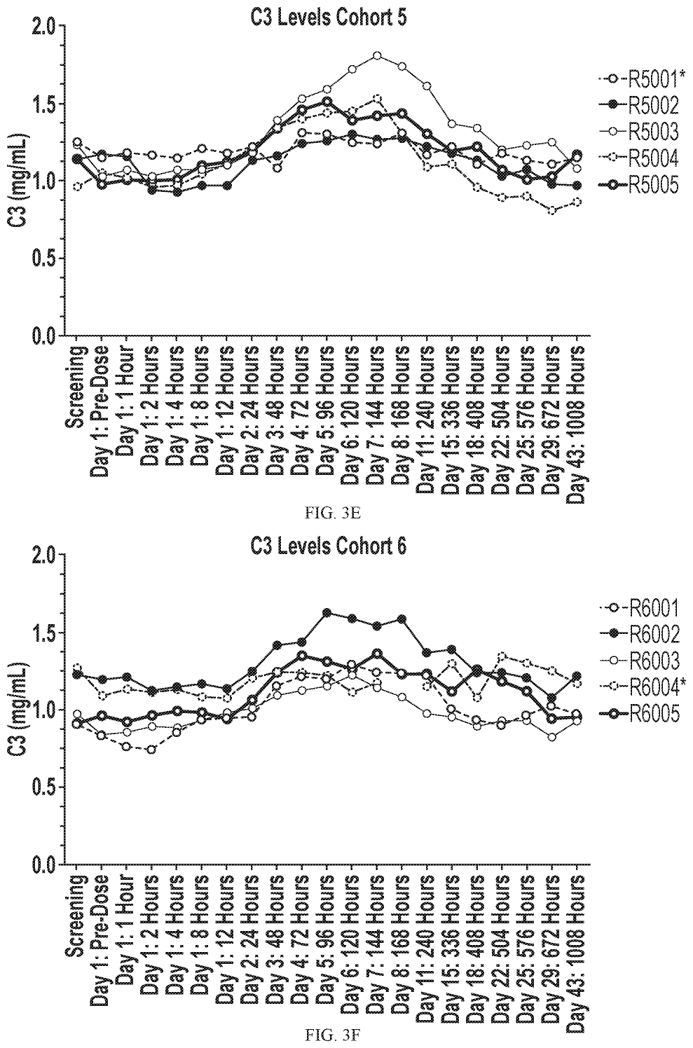

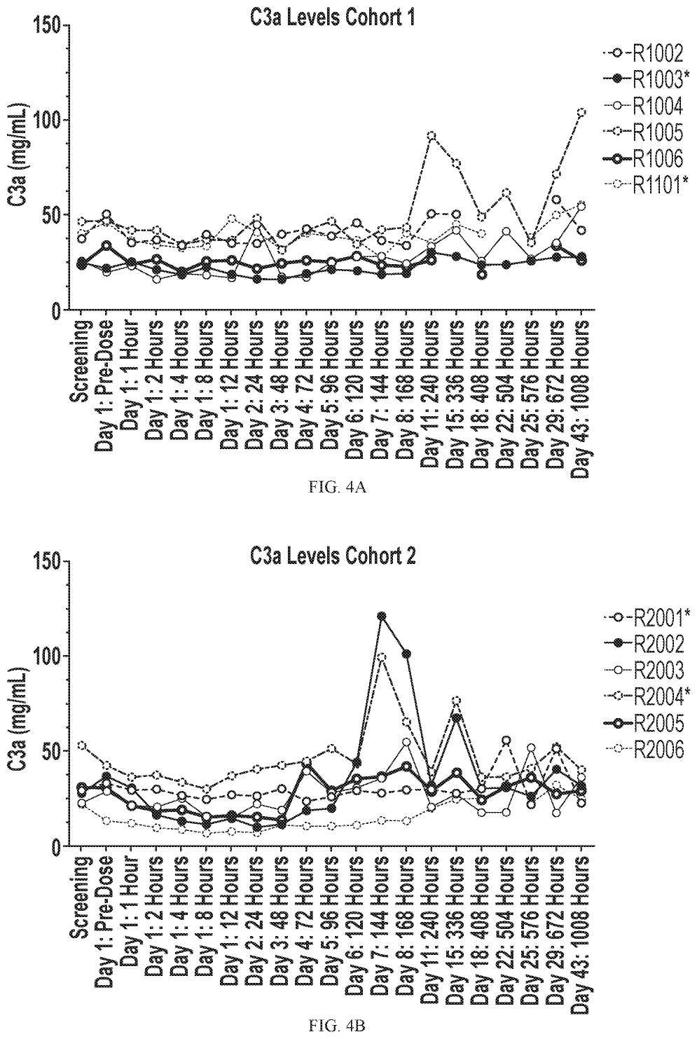

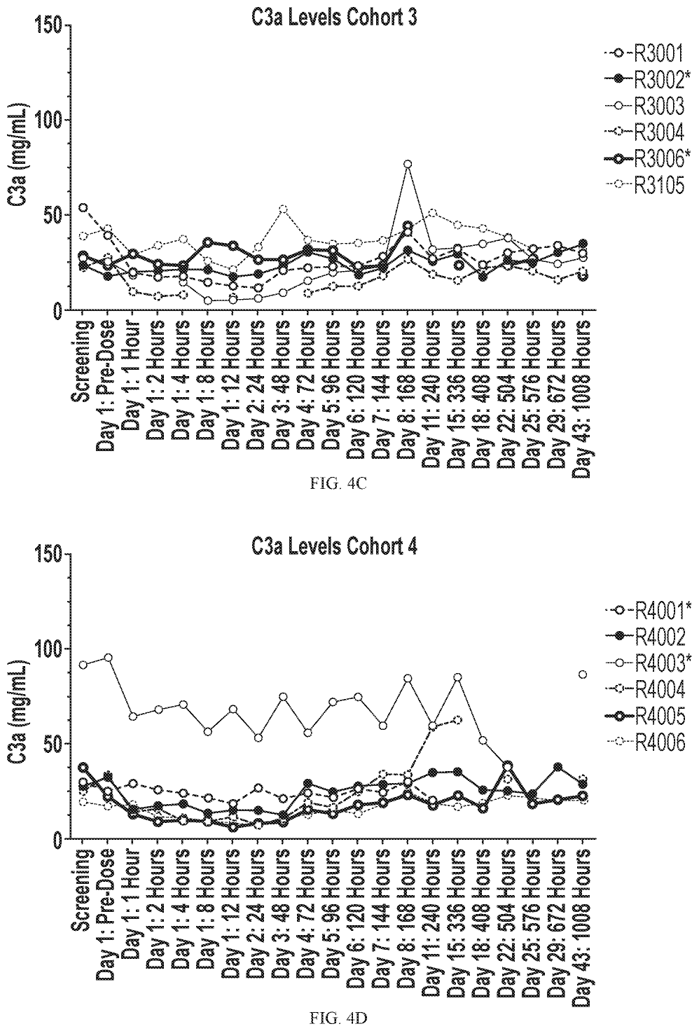

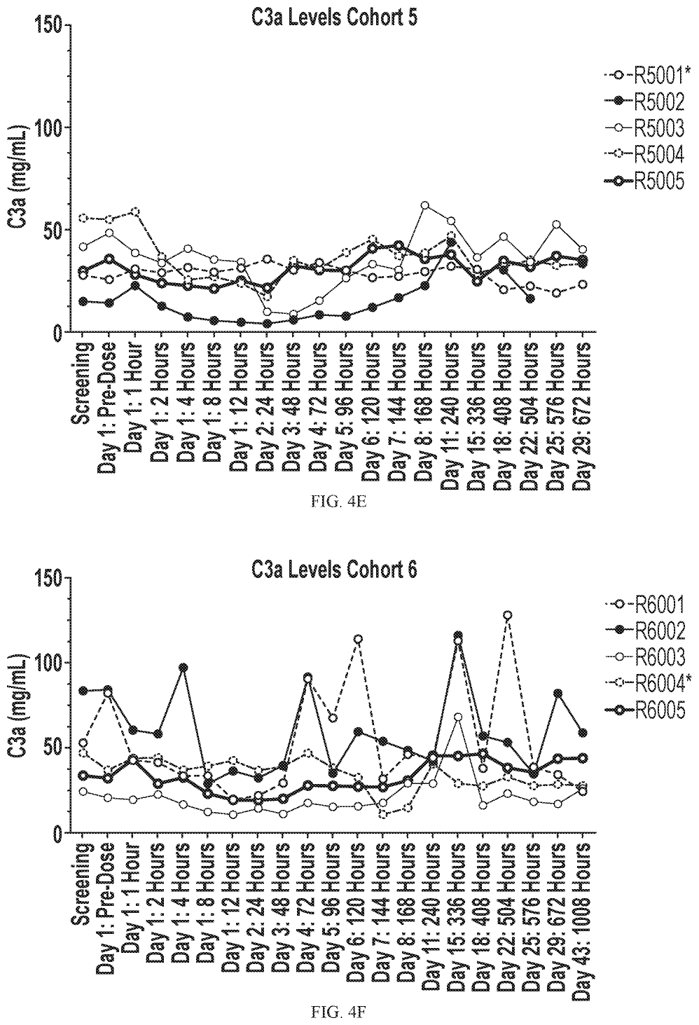

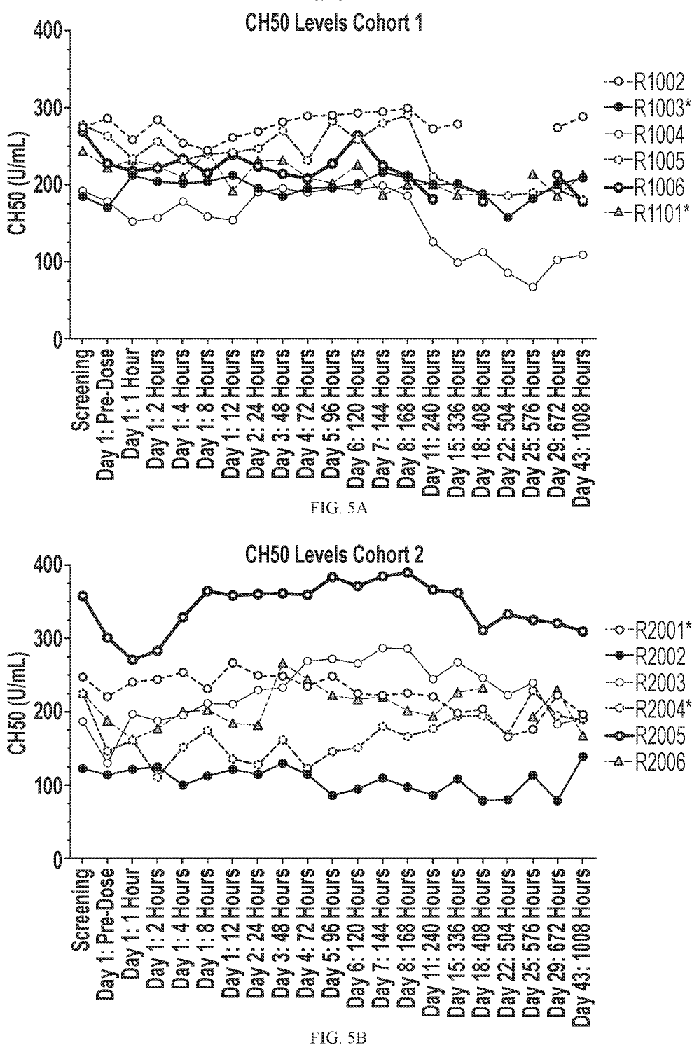

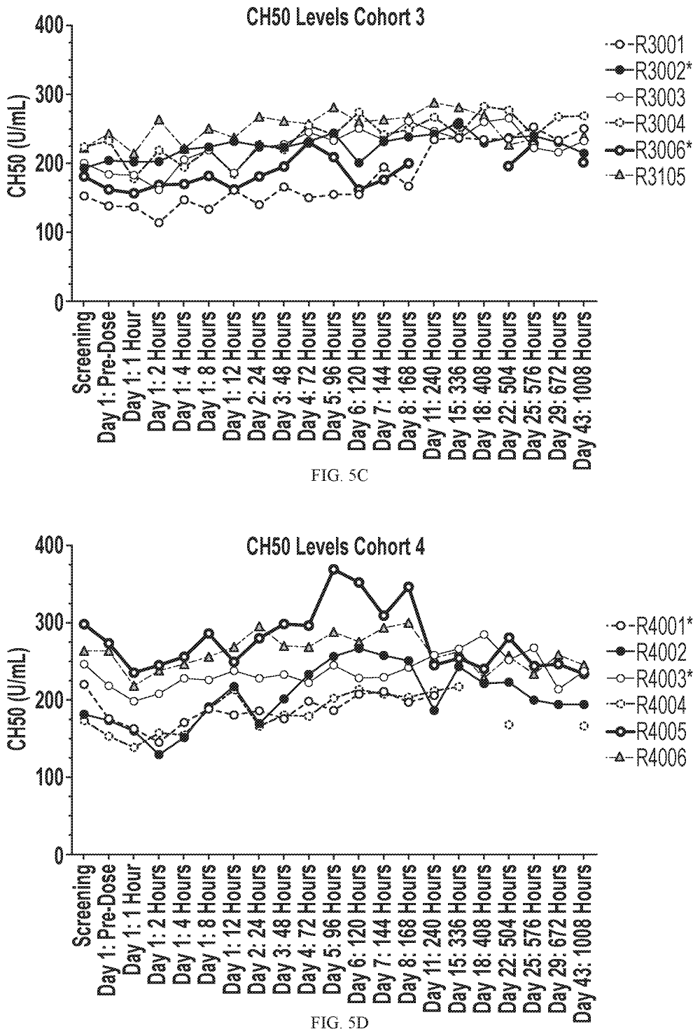

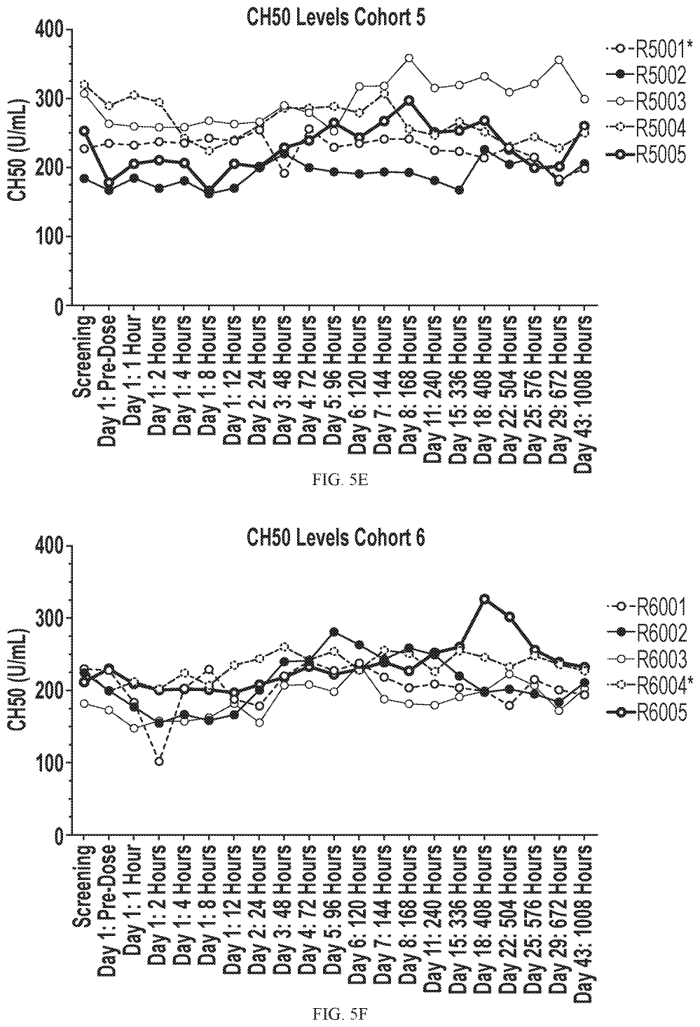

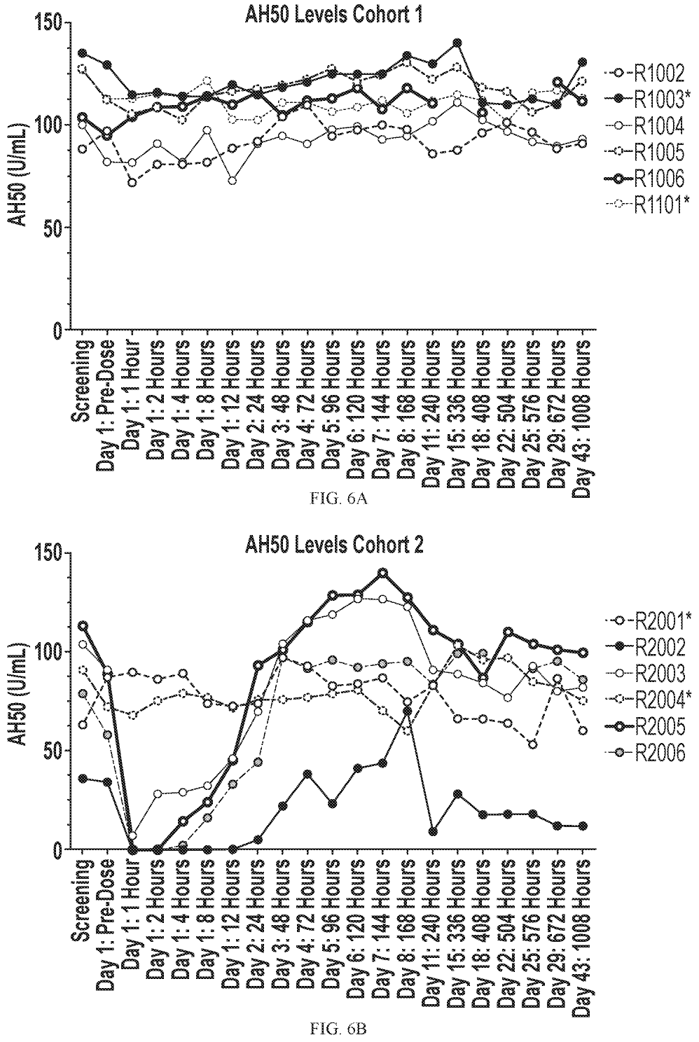

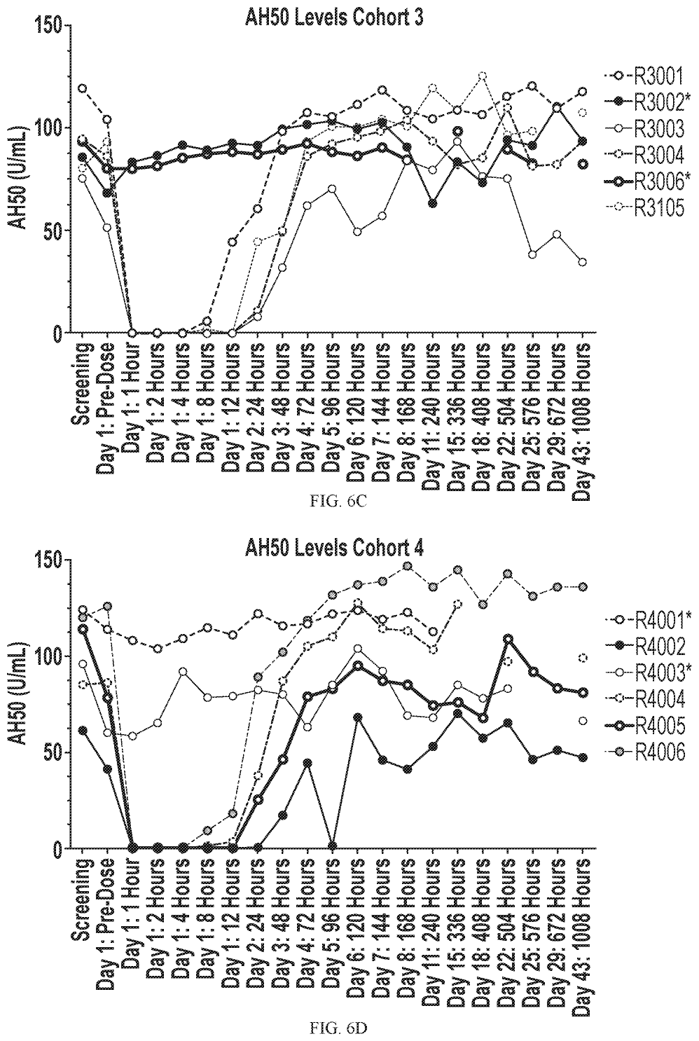

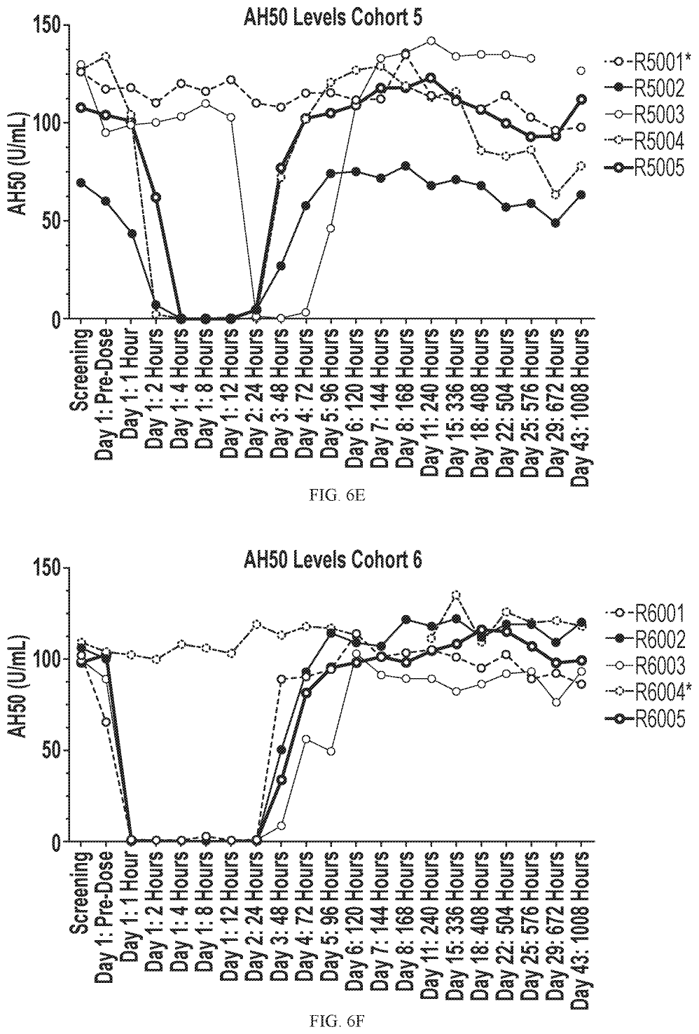

FIGS. 3A-3F depict C3 levels for the cohorts, where (*) indicate subjects who received placebo. FIGS. 4A-4F depict C3a levels for the cohorts. FIGS. 5A-5F depict CH50 levels for the cohorts. FIGS. 6A-6F depict AH50 levels for the cohorts.

Decreases in AH50 and concomitant increases in C3 levels in subjects suggest reduced consumption of C3 due to inhibition of the complement pathway by the PEGylated compstatin analog. The degree and duration of the observed changes in C3 and AH50 levels was variable across all cohorts. CH50 levels were largely unaffected following dosing. Without wishing to be bound by theory, this could have been due to the PEGylated compstatin analog not affecting the classical pathway as strongly, or could have been due to a lower sensitivity of the CH50 assay used, relative to the AH50 assay, based on the high dilutions of serum. There was variability in C3a levels following dosing of the PEGylated compstatin analog, which could potentially be attributed to the rapid clearance of C3a and binding to the C3a receptor.

These data demonstrate that the PEGylated compstatin analog controlled complement through modulation of C3 within 1 hour of administration, which lasted up to 12 hours after the end of the infusion. Further, multiple doses tested were able to completely suppress the AH50 hemolytic activity.

### Example 2—Pharmacology of Exemplary PEGylated Compstatin Analog Described in Example 1

### Binding to C3 and C3b

The binding of the PEGylated compstatin analog (described in Example 1) to C3 and C3b was assessed using isothermal titration calorimetry, where the compstatin analog was injected into a chamber with dialyzed human C3 or C3b protein. The Kd was calculated following the measurement of binding enthalpy.

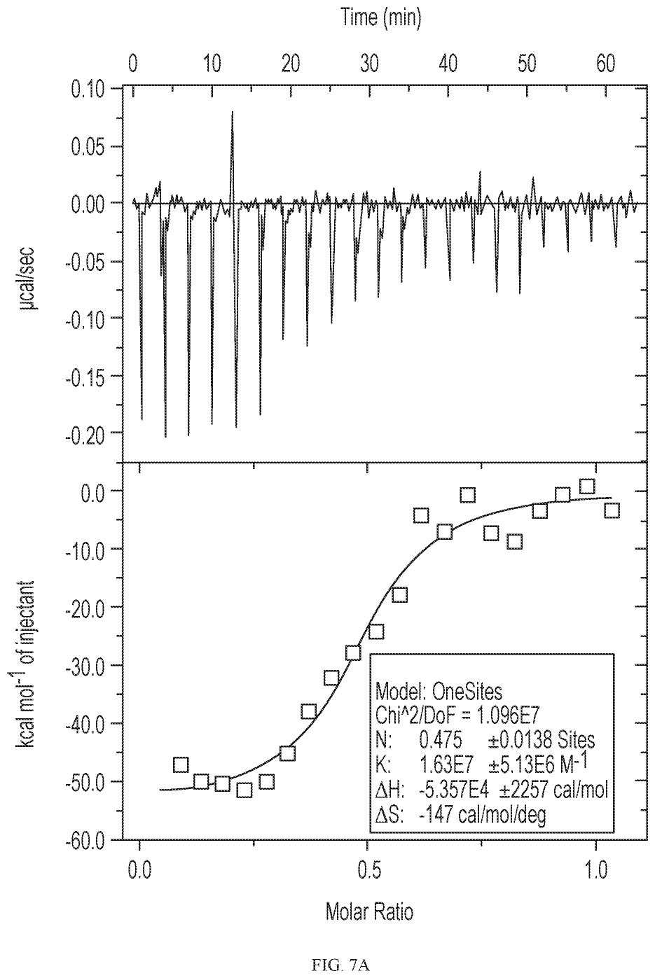

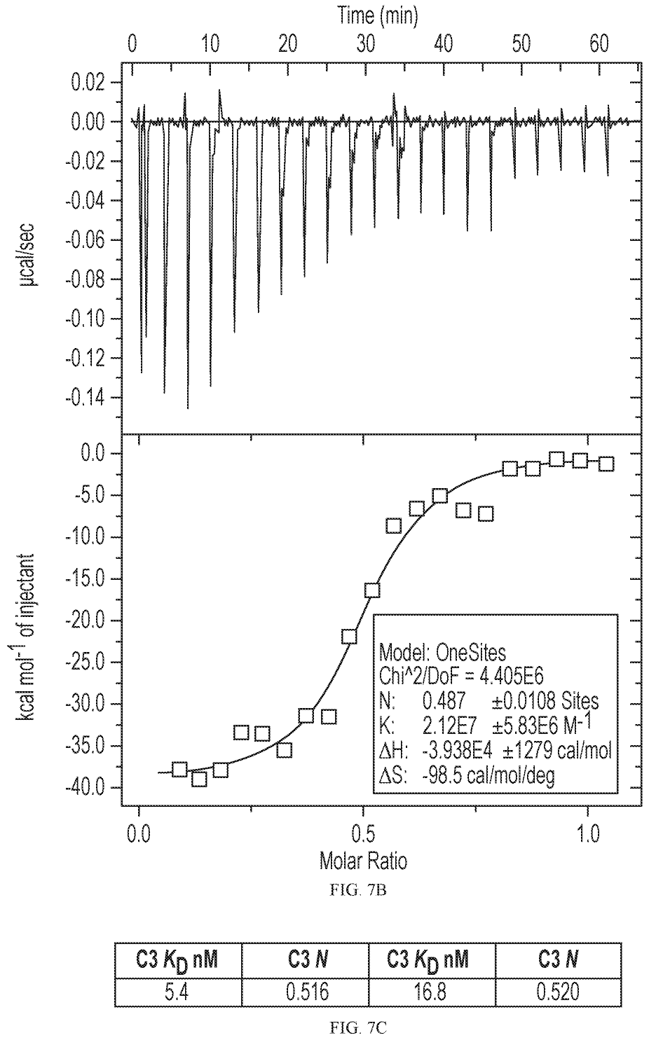

As shown in FIG. 7, the compstatin analog was shown to bind to C3 and C3b, with affinities of 5.4 and 16.8 nM, respectively. Notably, the binding stoichiometry between the compstatin analog and C3/C3b was 0.5, indicating that 1 molecule of the compstatin analog could associate with two C3 or C3b molecules, one for each peptide domain.

### Complement Inhibitory Activity

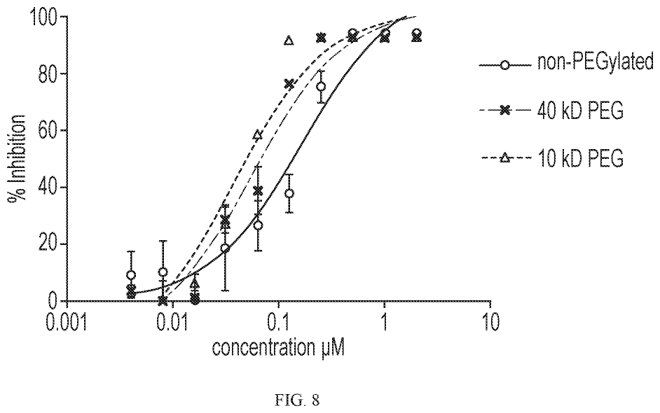

The in vitro bioactivity of the compstatin analog was assessed in an ELISA-based Wieslab assay. Human serum pre-incubated with varied concentrations of the compstatin analog was applied to microwells containing immobilized activators of one complement pathway: classical, or alternative. Complement activation was determined by the detection of a neo-epitope on the terminal complement complex, C5b-9, and measured in terms of optical density (FIG. 8).

As shown in FIG. 8, the in-vitro inhibitory activity of the compstatin analog (EC₅₀ 37 nM) was comparable to the corresponding compstatin analog having a PEG of about 40 kD (EC₅₀˜57 nM) and to the corresponding non-PEGylated peptide sequence (EC₅₀˜165 nM).

### Species Specificity

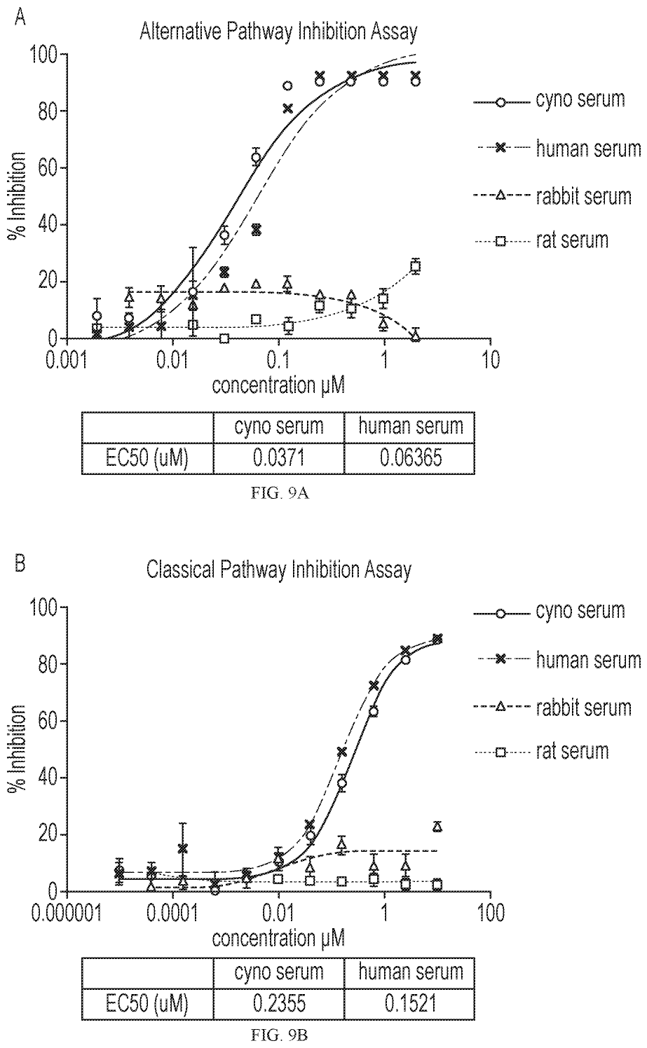

Inhibitory activity of the compstatin analog was tested in the serum of various primate and non-primate species in classical and alternative complement pathway Wieslab ELISA-based assays. As expected, the compstatin analog inhibited classical and alternative pathway activation in the serum from humans and cynomolgus monkeys in a concentration-dependent manner but produced no significant inhibition of either pathway in representative non-primate species serum (FIG. 9).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of inhibiting complement in a subject, comprising administering to a subject in need thereof about 10 mg to about 1200 mg of a PEGylated compstatin analog comprising a PEG of about 10 kD, wherein complement is inhibited or reduced (e.g., to a level that is 50%, 40%, 30%, 20%, 10%, 5%, or lower, relative to a control level) for about 6 hours to about 24 hours after administration, and wherein complement is not inhibited or reduced (e.g., to a level that is 50%, 40%, 30%, 20%, 10%, 5%, or lower, relative to a control level) about 12 hours to about 36 hours after administration.

2. The method of claim 1, comprising administering a single dose of the PEGylated compstatin analog.

3. The method of claim 2, wherein the single dose is a bolus.

4. The method of claim 2, wherein the single dose is an infusion.

5. The method of claim 3 or 4, comprising administering about 30 mg, about 90 mg, about 270 mg, about 540 mg, or about 600 mg of the PEGylated compstatin analog.

6. The method of claim 4 or 5, comprising administering the infusion at a rate of about 0.25 mg/min to about 45 mg/min.

7. The method of claim 5, comprising administering the infusion at a rate of about 1 mg/min, about 3 mg/min, about 9 mg/min, about 18 mg/min, or about 20 mg/min.

8. The method of any one of claims 4-7, comprising administering the infusion over a period of about 15 minutes to about 48 hours.

9. The method of claim 4, comprising administering about 30 mg of the PEGylated compstatin analog at an infusion rate of about 1 mg/min for about 30 minutes.

10. The method of claim 4, comprising administering about 90 mg of the PEGylated compstatin analog at an infusion rate of about 3 mg/min for about 30 minutes.

11. The method of claim 4, comprising administering about 270 mg of the PEGylated compstatin analog at an infusion rate of about 9 mg/min for about 30 minutes.

12. The method of claim 4, comprising administering about 540 mg of the PEGylated compstatin analog at an infusion rate of about 18 mg/min for about 30 minutes.

13. The method of claim 4, comprising administering about 540 mg of the PEGylated compstatin analog at an infusion rate of about 0.75 mg/min for about 12 hours.

14. The method of claim 4, comprising administering about 600 mg of the PEGylated compstatin analog at an infusion rate of about 20 mg/min for about 30 minutes.

15. The method of claim 1, comprising administering two or more doses of the PEGylated compstatin analog.

16. The method of claim 15, comprising administering a first dose (loading) and a second dose (maintenance).

17. The method of claim 16, wherein the first dose and the second dose comprise the same amount of the PEGylated compstatin analog.

18. The method of claim 16, wherein the first dose and the second dose comprise different amounts of the PEGylated compstatin analog.

19. The method of any one of claims 16-18, wherein the first dose comprises about 10 mg to about 600 mg of the PEGylated compstatin analog and the second dose comprises about 10 mg to about 600 mg of the PEGylated compstatin analog.

20. The method of any one of claims 16-19, wherein the first dose comprises about 30 mg, about 90 mg, about 240 mg, about 270 mg, about 360 mg, about 540 mg, or about 600 mg of the PEGylated compstatin analog.

21. The method of any one of claims 16-19, wherein the second dose comprises about 30 mg, about 90 mg, about 240 mg, about 270 mg, about 360 mg, about 540 mg, or about 600 mg of the PEGylated compstatin analog.

22. The method of any one of claims 16-19, wherein the first dose and the second dose comprise about 30 mg, about 90 mg, about 240 mg, about 270 mg, about 360 mg, about 540 mg, or about 600 mg of the PEGylated compstatin analog.

23. The method of any one of claims 16-22, wherein the first dose is a bolus, and the second dose is a bolus.

24. The method of any one of claims 16-22, wherein the first dose is a bolus, and the second dose is an infusion.

25. The method of any one of claims 16-22, wherein the first dose is an infusion, and the second dose is an infusion.

26. The method of any one of claims 16-22, wherein the first dose is an infusion and the second dose is a bolus.

27. The method of any claim 24 or 25, comprising administering the second dose at an infusion rate of about 0.25 mg/min to about 45 mg/min.

28. The method of claim 24 or 25, comprising administering the second dose at an infusion rate of about 0.25 mg/min, about 1 mg/min, about 3 mg/min, about 9 mg/min, about 16 mg/min, about 18 mg/min, or about 20 mg/min.

29. The method of claim 24 or 25, comprising administering the second dose over a period of about 15 minutes to about 48 hours.

30. The method of claim 24 or 25, comprising administering the second dose at about 30 mg of the PEGylated compstatin analog at an infusion rate of about 1 mg/min for about 30 minutes.

31. The method of claim 24 or 25, comprising administering the second dose at about 90 mg of the PEGylated compstatin analog at an infusion rate of about 3 mg/min for about 30 minutes.

32. The method of claim 24 or 25, comprising administering the second dose at about 240 mg of the PEGylated compstatin analog at an infusion rate of about 16 mg/min for about 15 minutes.

33. The method of claim 24 or 25, comprising administering the second dose at about 270 mg of the PEGylated compstatin analog at an infusion rate of about 9 mg/min for about 30 minutes.

34. The method of claim 24 or 25, comprising administering the second dose at about 360 mg of the PEGylated compstatin analog at an infusion rate of about 0.25 mg/min for about 24 hours.

35. The method of claim 24 or 25, comprising administering the second dose at about 540 mg of the PEGylated compstatin analog at an infusion rate of about 18 mg/min for about 30 minutes.

36. The method of claim 24 or 25, comprising administering the second dose at about 540 mg of the PEGylated compstatin analog at an infusion rate of about 0.75 mg/min for about 12 hours.

37. The method of claim 24 or 25, comprising administering the second dose at about 600 mg of the PEGylated compstatin analog at an infusion rate of about 20 mg/min for about 30 minutes.

38. The method of any claim 25 or 26, comprising administering the first dose at an infusion rate of about 0.25 mg/min to about 45 mg/min.

39. The method of claim 25 or 26, comprising administering the first dose at an infusion rate of about 0.25 mg/min, about 1 mg/min, about 3 mg/min, about 9 mg/min, about 16 mg/min, about 18 mg/min, or about 20 mg/min.

40. The method of claim 25 or 26, comprising administering the first dose over a period of about 15 minutes to about 48 hours.

41. The method of claim 25 or 26, comprising administering the first dose at about 30 mg of the PEGylated compstatin analog at an infusion rate of about 1 mg/min for about 30 minutes.

42. The method of claim 25 or 26, comprising administering the first dose at about 90 mg of the PEGylated compstatin analog at an infusion rate of about 3 mg/min for about 30 minutes.

43. The method of claim 25 or 26, comprising administering the first dose at about 240 mg of the PEGylated compstatin analog at an infusion rate of about 16 mg/min for about 15 minutes.

44. The method of claim 25 or 26, comprising administering the first dose at about 270 mg of the PEGylated compstatin analog at an infusion rate of about 9 mg/min for about 30 minutes.

45. The method of claim 25 or 26, comprising administering the first dose at about 360 mg of the PEGylated compstatin analog at an infusion rate of about 0.25 mg/min for about 24 hours.

46. The method of claim 25 or 26, comprising administering the first dose at about 540 mg of the PEGylated compstatin analog at an infusion rate of about 18 mg/min for about 30 minutes.

47. The method of claim 25 or 26, comprising administering the first dose at about 540 mg of the PEGylated compstatin analog at an infusion rate of about 0.75 mg/min for about 12 hours.

48. The method of claim 25 or 26, comprising administering the first dose at about 600 mg of the PEGylated compstatin analog at an infusion rate of about 20 mg/min for about 30 minutes.

49. The method of claim 25, comprising administering the first dose and the second dose at an infusion rate of about 0.25 mg/min to about 45 mg/min.

50. The method of claim 25, comprising administering the first dose and the second dose at an infusion rate of about 0.25 mg/min, about 1 mg/min, about 3 mg/min, about 9 mg/min, about 16 mg/min, about 18 mg/min, or about 20 mg/min.

51. The method of claim 25, comprising administering the first dose and the second dose over a period of about 15 minutes to about 48 hours.

52. The method of claim 25, comprising administering the first dose and the second dose at about 30 mg of the PEGylated compstatin analog at an infusion rate of about 1 mg/min for about 30 minutes.

53. The method of claim 25, comprising administering the first dose and the second dose at about 90 mg of the PEGylated compstatin analog at an infusion rate of about 3 mg/min for about 30 minutes.

54. The method of claim 25, comprising administering the first dose and the second dose at about 240 mg of the PEGylated compstatin analog at an infusion rate of about 16 mg/mi for about 15 minutes.

55. The method of claim 25, comprising administering the first dose and the second dose at about 270 mg of the PEGylated compstatin analog at an infusion rate of about 9 mg/min for about 30 minutes.

56. The method of claim 25, comprising administering the first dose and the second dose at about 360 mg of the PEGylated compstatin analog at an infusion rate of about 0.25 mg/min for about 24 hours.

57. The method of claim 25, comprising administering the first dose and the second dose at about 540 mg of the PEGylated compstatin analog at an infusion rate of about 18 mg/mi for about 30 minutes.

58. The method of claim 25, comprising administering the first dose and the second dose at about 540 mg of the PEGylated compstatin analog at an infusion rate of about 0.75 mg/min for about 12 hours.

59. The method of claim 25, comprising administering the first dose and the second dose at about 600 mg of the PEGylated compstatin analog at an infusion rate of about 20 mg/mi for about 30 minutes.

60. The method of claim 25, comprising administering the first dose at about 240 mg of the PEGylated compstatin analog and the second dose at about 360 mg of the PEGylated compstatin analog.

61. The method of claim 25, comprising administering the first dose at about 240 mg of the PEGylated compstatin analog at an infusion rate of about 16 mg/min for about 15 minutes.

62. The method of claim 25, comprising administering the second dose at about 360 mg of the PEGylated compstatin analog at an infusion rate of about 0.25 mg/min for about 24 hours.

63. The method of claim 25, comprising administering the first dose at about 240 mg of the PEGylated compstatin analog at an infusion rate of about 16 mg/min for about 15 minutes and administering the second dose at about 360 mg of the PEGylated compstatin analog at an infusion rate of about 0.25 mg/min for about 24 hours.

64. The method of any one of claims 1-63, wherein complement inhibition is assessed by measuring level of complement activity in a serum sample of the subject.

65. The method of claim 64, wherein level of complement activity is measured using an alternative pathway assay, a classical pathway assay, or both.

66. The method of any one of claims 1-65, wherein the PEGylated compstatin analog comprises a PEG having at least two compstatin analog moieties attached thereto.

67. The method of claim 66, wherein the PEGylated compstatin analog comprises a linear PEG having a compstatin analog moiety attached to each end.

68. The method of claim 66 or 67, wherein each compstatin analog moiety comprises a cyclic peptide that comprises the amino acid sequence of one of SEQ ID NOs: 3-36, 37, 69, 70, 71, and 72.

69. The method of any one of claims 66-68, wherein the PEGylated compstatin analog comprises one or more PEG moieties attached to one or more compstatin analog moieties, wherein: each compstatin analog moiety comprises a cyclic peptide having an amino acid sequence as set forth in any of SEQ ID NOs:3-36, extended by one or more terminal amino acids at the N-terminus, C-terminus, or both, wherein one or more of the amino acids has a side chain comprising a primary or secondary amine and is separated from the cyclic peptide by a rigid or flexible spacer optionally comprising an oligo(ethylene glycol) moiety; and each PEG is covalently attached via a linking moiety to one or more compstatin analog moieties, and wherein the linking moiety comprises an unsaturated alkyl moiety, a moiety comprising a nonaromatic cyclic ring system, an aromatic moiety, an ether moiety, an amide moiety, an ester moiety, a carbonyl moiety, an imine moiety, a thioether moiety, and/or an amino acid residue.

70. The method of any one of claims 66-69, wherein each compstatin analog moiety comprises a cyclic peptide extended by one or more amino acids at the N-terminus, C-terminus, or both, wherein the one or more amino acids is separated from the cyclic portion of the peptide by a rigid or flexible spacer that comprises 8-amino-3,6-dioxaoctanoic acid (AEEAc) or 11-amino-3,6,9-trioxaundecanoic acid.

71. The method of any one of claims 66-69, wherein the cyclic peptide comprises the amino acid sequence of SEQ ID NO:28, and wherein the spacer comprises AEEAc.

72. The method of any one of claims 1-71, wherein the PEGylated compstatin analog comprises the structure depicted in FIG. 1.

73. The method of any one of claims 1-72, comprising administering the PEGylated compstatin analog to a subject who has experienced a stroke shortly after the onset of one or more stroke symptoms, e.g., within about 5 minutes, 10 minutes, 15 minutes, 30 minutes, 1 hour, 2 hours, 3 hours, or 4 hours of onset of one or more stroke symptoms.

74. The method of any one of claims 1-72, comprising administering the PEGylated compstatin analog to a subject (i) prior to the start of a dialysis procedure (e.g., about 2 hours, 1 hour, 30 minutes, or 15 minutes prior to the start of dialysis) and/or (ii) during the dialysis procedure, and/or (iii) after the end of the dialysis procedure (e.g., for about 15 minutes, 30 minutes, 1 hour, or 2 hours after the end of the dialysis procedure).

75. The method of any one of claims 1-72, comprising administering the PEGylated compstatin analog to a subject who exhibits a sign or symptom of, or is diagnosed as having, a microangiopathy.

76. The method of claim 75, wherein the PEGylated compstatin analog is administered for about 6 hours, 12 hours, 18 hours, 24 hours, 2 days, 3 days, or more.

77. The method of any one of claims 1-72, comprising administering the PEGylated compstatin analog to a subject who has or is at risk of developing autoimmune encephalitis.

78. The method of any one of claims 1-72, comprising administering the PEGylated compstatin analog to a subject who has received or is receiving an AAV viral vector.

79. The method of claim 78, comprising administering the PEGylated compstatin analog to a subject prior to receiving an AAV viral vector.

80. The method of claim 78, comprising administering a first dose and a second dose of the PEGylated compstatin analog, wherein the second dose is administered concurrently with an AAV viral vector.
